SEQUENCE LISTING (97 sequences)

SEQ ID NO: 1 - protein, 187 aa, Artificial Sequence
  wild-type murine dihydrofolate reductase
MVRPLNCIVAVSQNMGIGKNGDLPWPPLRNEFKYFQRMTTTSSVEGKQNLVIMGRKTWFSIPEKNRPLKDRINIVLSRELKEPPRGAHFLAKSLDDALRLIEQPELASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 2 - protein, 187 aa, Artificial Sequence
  engineered murine dihydrofolate reductase
MVRPLNCIVAVSQNMGIGKNGDLPWPPLRNESKYFQRMTQTDSVESKQNLVIMGRKTWFSIPESNRPLKDRINIVLSQELKEPPRGAHFLAKSLDDALRLIESPELASKVDSVWIVGGSSVYQEAMTQPGHLRLFVTQIMQEFESDTFFPEIDSGKYKLLPESPGVLSQVQEEKGIKYKFEVYEKKD

SEQ ID NO: 3 - protein, 187 aa, Artificial Sequence
  wild-type human dihydrofolate reductase
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 4 - DNA, 564 bp, Artificial Sequence
  engineered murine dihydrofolate reductase
atggttcgaccattgaactgcatcgtcgccgtgagtcagaatatggggattggcaagaacggagacctaccctggcctccgctcaggaatgagtcaaagtacttccaaagaatgactcagactgactcagttgagtcaaaacagaatctggtgattatgggtaggaaaacctggttctccattcctgagtcaaatcgacctttaaaggacagaattaatatagttctgagtcaagaactcaaagaaccaccacgaggagctcattttcttgccaaaagtttggatgatgccttaagacttattgagtcaccggaattggcgagcaaagttgactcagtttggatcgtcggaggcagttctgtttaccaggaagccatgactcaaccaggccaccttagactctttgtgactcagatcatgcaggaatttgagtcagacacgtttttcccagaaattgactcagggaaatataaacttctccctgagtcaccaggcgtcctgagtcaggtccaggaggaaaaaggcatcaagtataagtttgaagtctacgagaagaaagactaa

SEQ ID NO: 5 - DNA, 7 bp, Artificial Sequence
  TPA response elements (TRE)
tgactca

SEQ ID NO: 6 - DNA, 7 bp, Artificial Sequence
  TPA response elements (TRE)
tgagtca

SEQ ID NO: 7 - DNA, 6 bp, Artificial Sequence
  Ebox response elements
cacgtg

SEQ ID NO: 8 - DNA, 6 bp, Artificial Sequence
  Ebox response elements
cacatg

SEQ ID NO: 9 - DNA, 10 bp, Artificial Sequence
  C/EBP protein response element
attgcgcaat

SEQ ID NO: 10 - DNA, 8 bp, Artificial Sequence
  cAMP response element (CRE)
tgacgtca

SEQ ID NO: 11 - DNA, 567 bp, Artificial Sequence
  polynucleotide encoding engineered murine dihydrofolate reductase including restriction enzyme sites
gctagcgttcgaccattgaactgcatcgtcgccgtgagtcagaatatggggattggcaagaacggagacctaccctggcctccgctcaggaatgagtcaaagtacttccaaagaatgactcagactgactcagttgagtcaaaacagaatctggtgattatgggtaggaaaacctggttctccattcctgagtcaaatcgacctttaaaggacagaattaatatagttctgagtcaagaactcaaagaaccaccacgaggagctcattttcttgccaaaagtttggatgatgccttaagacttattgagtcaccggaattggcgagcaaagttgactcagtttggatcgtcggaggcagttctgtttaccaggaagccatgactcaaccaggccaccttagactctttgtgactcagatcatgcaggaatttgagtcagacacgtttttcccagaaattgactcagggaaatataaacttctccctgagtcaccaggcgtcctgagtcaggtccaggaggaaaaaggcatcaagtataagtttgaagtctacgagaagaaagactaa

SEQ ID NO: 12 - DNA, 7 bp, Artificial Sequence
  example reading frames
tgactca

SEQ ID NO: 13 - DNA, 7 bp, Artificial Sequence
  example reading frames
tgactca

SEQ ID NO: 14 - DNA, 7 bp, Artificial Sequence
  example reading frames
tgactca

SEQ ID NO: 15 - protein, 15 aa, Artificial Sequence
  example reading frame 3
FSYCLPHRITNVADG

SEQ ID NO: 16 - DNA, 9 bp, Artificial Sequence
  example codon triplets containing TREs
gtgagtcag

SEQ ID NO: 17 - DNA, 9 bp, Artificial Sequence
  example codon triplets containing TREs
aatgagtca

SEQ ID NO: 18 - DNA, 9 bp, Artificial Sequence
  example codon triplets containing TREs
atgactcag

SEQ ID NO: 19 - DNA, 9 bp, Artificial Sequence
  example codon triplets containing TREs
actgactca

SEQ ID NO: 20 - DNA, 9 bp, Artificial Sequence
  example codon triplets containing TREs
gttgagtca

SEQ ID NO: 21 - DNA, 9 bp, Artificial Sequence
  example codon triplets containing TREs
cctgagtca

SEQ ID NO: 22 - DNA, 9 bp, Artificial Sequence
  example codon triplets containing TREs
ctgagtcaa

SEQ ID NO: 23 - DNA, 9 bp, Artificial Sequence
  example codon triplets containing TREs
attgagtca

SEQ ID NO: 24 - DNA, 9 bp, Artificial Sequence
  example codon triplets containing TREs
gttgactca

SEQ ID NO: 25 - DNA, 9 bp, Artificial Sequence
  example codon triplets containing TREs
atgactcaa

SEQ ID NO: 26 - DNA, 9 bp, Artificial Sequence
  example codon triplets containing TREs
gtgactcag

SEQ ID NO: 27 - DNA, 9 bp, Artificial Sequence
  example codon triplets containing TREs
tttgagtca

SEQ ID NO: 28 - DNA, 9 bp, Artificial Sequence
  example codon triplets containing TREs
attgactca

SEQ ID NO: 29 - DNA, 9 bp, Artificial Sequence
  example codon triplets containing TREs
cctgagtca

SEQ ID NO: 30 - DNA, 9 bp, Artificial Sequence
  example codon triplets containing TREs
ctgagtcag

SEQ ID NO: 31 - protein, 189 aa, Artificial Sequence
  engineered murine dihydrofolate reductase used during design process
ASVRPLNCIVAVSQNMGIGKNGDLPWPPLRNESKYFQRMTQTDSVESKQNLVIMGRKTWFSIPESNRPLKDRINIVLSQELKEPPRGAHFLAKSLDDALRLIESPELASKVDSVWIVGGSSVYQEAMTQPGHLRLFVTQIMQEFESDTFFPEIDSGKYKLLPESPGVLSQVQEEKGIKYKFEVYEKKDA

SEQ ID NO: 32 - DNA, 13 bp, Artificial Sequence
  Maf recognition elements (MAREs)
  misc_feature (7)..(7): n = G or C
tgctgantcagca

SEQ ID NO: 33 - DNA, 14 bp, Artificial Sequence
  Maf recognition elements (MAREs)
  misc_feature (7)..(8): position 7 and 8 together are either GC or CG
tgctganntcagca

SEQ ID NO: 34 - DNA, 8 bp, Artificial Sequence
  Par/CREB-2/PAP binding site
ttacgtaa

SEQ ID NO: 35 - DNA, 9 bp, Artificial Sequence
  binding site
ggccaatct

SEQ ID NO: 36 - DNA, 570 bp, Artificial Sequence
  an exemplary murine dihydrofolate reductase gene engineered to include CRE binding sites
atggttcgaccattgaactgcatcgtcgccgtgtcccaaaatatggggattggcaagaacggagacctaccctggcctccgctcaggaacgagttcaagtacttccaaagaatgacgtcaacctcttcagtggaaggtaaacagaatctggtgattatgggtaggaaaacctggttctccattcctgagaagaatcgacctttaaaggacagaattaatatagtgacgtcaagagaactcaaagaaccaccacgaggagctcattttcttgccaaaagtttggatgatgccttaagacttattgaacaaccggaattgacgtcaaaagtagacatggtttggatcgtcggaggcagttctgtttaccaggaagccatgaatcaaccaggccaccttagactctttgtgacgtcaatcatgcaggaatttgaaagtgacacgtttttcccagaaattgatttggggaaatataaacttctcccagaatacccaggcgtgacgtcagaggtccaggaggaaaaaggcatcaagtataagtttgaagtctacgagaagaaagactaagcttaa

SEQ ID NO: 37 - protein, 187 aa, Artificial Sequence
  an exemplary murine dihydrofolate reductase engineered to include CRE binding sites
MVRPLNCIVAVSQNMGIGKNGDLPWPPLRNEFKYFQRMTSTSSVEGKQNLVIMGRKTWFSIPEKNRPLKDRINIVTSRELKEPPRGAHFLAKSLDDALRLIEQPELTSKVDMVWIVGGSSVYQEAMNQPGHLRLFVTSIMQEFESDTFFPEIDLGKYKLLPEYPGVTSEVQEEKGIKYKFEVYEKKD

SEQ ID NO: 38 - DNA, 570 bp, Artificial Sequence
  an exemplary murine dihydrofolate reductase gene engineered to include CCAAT binding sites
atggttcgaccattgaactgcatcgtcgccgtgtcccaaaatatggggattggcaagaacggagacctaccctggcctccattgcgcaatgagttcaagtacttccaaagaatgaccacaacctcttcagtggaaggtaaacagaatctggtgattatgggtaggaaaacctggttctccattcctgagaagaatcgaccattgcgcaatagaattaatatagttctcagtagagaattgcgcaatccaccacgaggagctcattttattgcgcaatccttggatgatgcattgcgcaatattgaacaaccggaattggcgagcaaagtagacatggtttggatcgtcggaggcagttctgtttaccaggaagccatgaatcaaccaggccaccttagactctttgtgacaaggatcatgcaggaatttgaaagtgacacgtttttcccagaaattgatttggggaaatataaacttctcccagaatacccaggcgtcctctctgaattgcgcaatgaaaaaggcatcaagtataagtttgaagtctacgagaagaaagactaagcttaa

SEQ ID NO: 39 - protein, 187 aa, Artificial Sequence
  an exemplary murine dihydrofolate reductase engineered to include CCAAT binding sites
MVRPLNCIVAVSQNMGIGKNGDLPWPPLRNEFKYFQRMTTTSSVEGKQNLVIMGRKTWFSIPEKNRPLRNRINIVLSRELRNPPRGAHFIAQSLDDALRNIEQPELASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 40 - DNA, 570 bp, Artificial Sequence
  an exemplary murine dihydrofolate reductase gene engineered to include Eboxes
atggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggcgcacgtggttctccattcctgagaagaatcgacctttaaaggacagaattaatatagttctctcacgtgaactcaaagaaccaccacgtggagctcacgtgcttgccaaatcactggatgatgcattaagacttattgaacaaccggaattggcgtcacgtgtagacatggtttggatcgtcggaggcagttctgtttaccaggaagccatgaatcaaccaggccacgtgagactctttgtgacacgtgtcatgcaggaatttgaaagtgacacgtttttcccagaaattgatttggggaaatataaacttctcccagaatacccaggcgtcctctcacgtgtccaggaggaaaaaggcatcaagtataagtttgaagtctacgagaagaaagactaagcttaa

SEQ ID NO: 41 - protein, 187 aa, Artificial Sequence
  an exemplary murine dihydrofolate reductase engineered to include Eboxes
MVRPLNCIVAVSQNMGIGKNGDLPWPPLRNEFKYFQRMTTTSSVEGKQNLVIMGRRTWFSIPEKNRPLKDRINIVLSRELKEPPRGAHVLAKSLDDALRLIEQPELASRVDMVWIVGGSSVYQEAMNQPGHVRLFVTRVMQEFESDTFFPEIDLGKYKLLPEYPGVLSRVQEEKGIKYKFEVYEKKD

SEQ ID NO: 42 - DNA, 3553 bp, Artificial Sequence
  p300-mDHFR plasmid
ctcgagaaatcataaaaaatttatttgctttgtgagcggataacaattataatagattcaattgtgagcggataacaatttcacacagaattcattaaagaggagaaattaagcatgcaccatcaccatcaccatgctagcgttcgaccattgaactgcatcgtcgccgtgagtcagaatatggggattggcaagaacggagacctaccctggcctccgctcaggaatgagtcaaagtacttccaaagaatgactcagactgactcagttgagtcaaaacagaatctggtgattatgggtaggaaaacctggttctccattcctgagtcaaatcgacctttaaaggacagaattaatatagttctgagtcaagaactcaaagaaccaccacgaggagctcattttcttgccaaaagtttggatgatgccttaagacttattgagtcaccggaattggcgagcaaagttgactcagtttggatcgtcggaggcagttctgtttaccaggaagccatgactcaaccaggccaccttagactctttgtgactcagatcatgcaggaatttgagtcagacacgtttttcccagaaattgactcagggaaatataaacttctccctgagtcaccaggcgtcctgagtcaggtccaggaggaaaaaggcatcaagtataagtttgaagtctacgagaagaaagactaagcttaattagctgagcttggactcctgttgatagatccagtaatgacctcagaactccatctggatttgttcagaacgctcggttgccgccgggcgttttttattggtgagaatccaagctagtttgggaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccgcgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 43 - DNA, 3600 bp, Artificial Sequence
  p230d-basic-cJun plasmid
ctcgagaaatcataaaaaatttatttgctttgtgagcggataacaattataatagattcaattgtgagcggataacaatttcacacagaattcattaaagaggagaaattaagcatgcgcattaaagccgaacgcaaacggatgcgcaaccgcatcgcagcctccaagtgccgcaaacgcaaattggagcgcatcgcccgcttggaagaaaaggtgaaaaccctgaaagcacagaactatgagctggcctccaccgccaacatgttgcgcgaacaggtggcccagctcggcgcgcctcatcaccatcaccatcactgataaagcgcgccttgataagcttaattagctgagcttggactcctgttgatagatccagtaatgacctcagaactccatctggatttgttcagaacgctcggttgccgccgggcgttttttattggtgagaatccaggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 44 - DNA, 3740 bp, Artificial Sequence
  pREP4 expressing the lac repressor
aagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgacggtcgtttcgcatgcttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcgacaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtcaacgtaaatgcatgccgcttcgccttcgcgcgcgaattgtcgaccctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttaattctcatgtttgacagcttatcatcgat

SEQ ID NO: 45 - DNA, 3117 bp, Artificial Sequence
  control cJun plasmid lacking the DNA-binding basic region
ctcgagaaatcataaaaaatttatttgctttgtgagcggataacaattataatagattcaattgtgagcggataacaatttcacacagaattcattaaagaggagaaattaagcatgcaccatcaccatcaccatgctagcatcgcccggctggaggaaaaagtgaagaccttgaaggcccagaactatgagctggcgtccacggccaacatgctccgggaacaggtggcacagcttggcgcgccttaaggtagctctaagcttaattagctgagcttggactcctgttgatagatccagtaatgacctcagaactccatctggatttgttcagaacgctcggttgccgccgggcgttttttattggtgagaatccaggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 46 - DNA, 75 bp, Artificial Sequence
  basic-c-Jun
cgcattaaagccgaacgcaaacggatgcgcaaccgcatcgcagcctccaagtgccgcaaacgcaaattggagcgc

SEQ ID NO: 47 - protein, 25 aa, Artificial Sequence
  basic-c-Jun
RIKAERKRMRNRIAASKCRKRKLER

SEQ ID NO: 48 - DNA, 129 bp, Artificial Sequence
  ABeta1-42
gacgctgaatttcgccacgactccggctatgaggtacaccaccagaaactggttttttttgctgaggacgttggctccaacaaaggtgctatcatcggtctgatggttggcggcgttgttatcgcttaa

SEQ ID NO: 49 - protein, 42 aa, Artificial Sequence [sequence omitted]

SEQ ID NO: 50 - DNA, 207 bp, Artificial Sequence
  basic ABeta1-42
atgcgcattaaagccgaacgcaaacggatgcgcaaccgcatcgcagcctccaagtgccgcaaacgcaaattggagcgcgacgctgaatttcgccacgactccggctatgaggtacaccaccagaaactggttttttttgctgaggacgttggctccaacaaaggtgctatcatcggtctgatggttggcggcgttgttatcgcttaa

SEQ ID NO: 51 - protein, 68 aa, Artificial Sequence
  basic Abeta1-42
MRIKAERKRMRNRIAASKCRKRKLERDAEFRHDSGYEVHHQKLVFFAEDVGSNKGAIIGLMVGGVVIA

SEQ ID NO: 52 - DNA, 420 bp, Artificial Sequence
  AlphaS
gatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 53 - protein, 139 aa, Artificial Sequence
  AlphaS
DVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 54 - DNA, 501 bp, Artificial Sequence
  basic AlphaS
atgcgcattaaagccgaacgcaaacggatgcgcaaccgcatcgcagcctccaagtgccgcaaacgcaaattggagcgcgatgtgtttatgaaaggtctgagcaaagcgaaagaaggcgtggtggctgcggcggaaaaaacgaaacagggcgtggcggaagcggccggcaaaacgaaagaaggtgttctgtatgtcggcagcaaaaccaaagaaggcgtggttcatggtgtggccaccgttgcagaaaaaacgaaagaacaggtcaccaacgtgggcggtgctgtcgtgaccggtgttacggctgtcgcgcaaaaaacggtggaaggcgcgggttctattgcggcggcaaccggtttcgttaaaaaagatcagctgggtaaaaatgaagaaggcgcgccgcaagaaggtatcctggaagacatgccggtggatccggacaacgaagcgtatgaaatgccgtcggaagaaggctatcaagactatgaaccggaagcgtaatga

SEQ ID NO: 55 - protein, 165 aa, Artificial Sequence
  basic AlphaS
MRIKAERKRMRNRIAASKCRKRKLERDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 56 - DNA, 10 bp, Artificial Sequence
  binding site
  misc_feature (3)..(8): n = A or T
ccnnnnnngg

SEQ ID NO: 57 - DNA, 6 bp, Artificial Sequence
  binding site
caggtg

SEQ ID NO: 58 - DNA, 6 bp, Artificial Sequence
  binding site
cacctg

SEQ ID NO: 59 - DNA, 6 bp, Artificial Sequence
  binding site
  misc_feature (3)..(3): n = A or T
tgnggg

SEQ ID NO: 60 - DNA, 8 bp, Artificial Sequence
  binding site
tcacacct

SEQ ID NO: 61 - DNA, 6 bp, Artificial Sequence
  binding site
tataaa

SEQ ID NO: 62 - DNA, 14 bp, Artificial Sequence
  binding site
gttggcatggcaac

SEQ ID NO: 63 - DNA, 10 bp, Artificial Sequence
  binding site
  misc_feature (1)..(1): n = A or G
  misc_feature (8)..(9): n = A or G
  misc_feature (10)..(10): n = C or T
nctaaccnnn

SEQ ID NO: 64 - DNA, 6 bp, Artificial Sequence
  binding site
aaatat

SEQ ID NO: 65 - DNA, 6 bp, Artificial Sequence
  binding site
  misc_feature (1)..(1): n = A, C or G
  misc_feature (6)..(6): n = C, G or T
ncgcgn

SEQ ID NO: 66 - DNA, 9 bp, Artificial Sequence
  binding site
taccgacat

SEQ ID NO: 67 - DNA, 19 bp, Artificial Sequence
  binding site
gataatgataatcattatc

SEQ ID NO: 68 - DNA, 10 bp, Artificial Sequence
  binding site
gccacgtggc

SEQ ID NO: 69 - DNA, 7 bp, Artificial Sequence
  binding site
agccgcc

SEQ ID NO: 70 - DNA, 6 bp, Artificial Sequence
  binding site
acacca

SEQ ID NO: 71 - DNA, 7 bp, Artificial Sequence
  binding site
tgtaaag

SEQ ID NO: 72 - DNA, 6 bp, Artificial Sequence
  binding site
cctttt

SEQ ID NO: 73 - DNA, 10 bp, Artificial Sequence
  binding site
atttactaac

SEQ ID NO: 74 - protein, 4 aa, Artificial Sequence
  peptide linker
GGGS

SEQ ID NO: 75 - protein, 5 aa, Artificial Sequence
  peptide linker
GGGGS

SEQ ID NO: 76 - protein, 4 aa, Artificial Sequence
  peptide linker
GGSG

SEQ ID NO: 77 - protein, 4 aa, Artificial Sequence
  peptide linker
GSGG

SEQ ID NO: 78 - protein, 4 aa, Artificial Sequence
  peptide linker
SGGG

SEQ ID NO: 79 - protein, 4 aa, Artificial Sequence
  peptide linker
SSGG

SEQ ID NO: 80 - protein, 4 aa, Artificial Sequence
  peptide linker
SSSG

SEQ ID NO: 81 - protein, 11 aa, Artificial Sequence
  peptide linker
TGGGGSGGGGS

SEQ ID NO: 82 - protein, 15 aa, Artificial Sequence
  synthetic construct
FLIVSPTAYHNDCRG

SEQ ID NO: 83 - protein, 7 aa, Artificial Sequence
  synthetic construct
IMTNKSR

SEQ ID NO: 84 - protein, 4 aa, Artificial Sequence
  synthetic construct
YHND

SEQ ID NO: 85 - protein, 6 aa, Artificial Sequence
  synthetic construct
FLSYCW

SEQ ID NO: 86 - protein, 12 aa, Artificial Sequence
  synthetic construct
LIVSPTAQKERG

SEQ ID NO: 87 - protein, 4 aa, Artificial Sequence
  synthetic construct
PLRN

SEQ ID NO: 88 - protein, 4 aa, Artificial Sequence
  synthetic construct
PLKD

SEQ ID NO: 89 - protein, 4 aa, Artificial Sequence
  synthetic construct
ELKE

SEQ ID NO: 90 - protein, 4 aa, Artificial Sequence
  synthetic construct
ELRN

SEQ ID NO: 91 - protein, 4 aa, Artificial Sequence
  synthetic construct
LAKS

SEQ ID NO: 92 - protein, 4 aa, Artificial Sequence
  synthetic construct
IAQS

SEQ ID NO: 93 - protein, 4 aa, Artificial Sequence
  synthetic construct
ALRL

SEQ ID NO: 94 - protein, 4 aa, Artificial Sequence
  synthetic construct
ALRN

SEQ ID NO: 95 - protein, 4 aa, Artificial Sequence
  synthetic construct
EVQE

SEQ ID NO: 96 - protein, 16 aa, Artificial Sequence
  synthetic construct
FLIVSPTAYHNDCRSG

SEQ ID NO: 97 - DNA, 15 bp, Artificial Sequence
  Maf recognition elements (MAREs)
tgctgagcgtcagca

(19) United States
(12) Patent Application Publication
(10) Pub. No.: US 2023/0146038 A1
(43) Pub. Date: May 11, 2023

(54) DIMERIZATION ASSAY

(71) Applicant: The University of Bath, Bath Somerset (GB)

(72) Inventors: Jody Michael MASON, Bath Somerset (GB); Neil Mark KAD, Kent (GB)

(73) Assignee: The University of Bath, Bath Somerset (GB)

(21) Appl. No.: 17/796,156

(22) PCT Filed: Feb. 3, 2021
(86) PCT No.: PCT/EP2021/052568

§ 371 Date: Jul. 28, 2022

(30) Foreign Application Priority Data
Apr. 2, 2020 (GB) 2001491.6

(51) Int. Cl. C12N 15/10

(52) U.S. Cl. CPC C12N 15/1055

(57) ABSTRACT

Disclosed are methods, kits and cells for screening an inhibitor of association between candidate binding partners, such as for screening antagonists of amyloid peptides. The methods, kits and cells employ a reporter expression cassette and hybrid proteins. The reporter expression cassette encodes a reporter and comprises at least one DNA binding site. Each hybrid protein comprises a candidate binding partner and a component of a DNA binding protein and, upon association, form a DNA-binding complex capable of binding to the at least one binding site and inhibiting expression of the reporter. The methods, kits and cells find application, for example, in the identification of inhibitors that may be useful in treating diseases associated with protein aggregation, such as Alzheimer's Disease and Parkinson's Disease.

### CROSS-REFERENCE

This application is a 371 National Stage filing and claims the benefit under 35 U.S.C. § 120 to International Application No. PCT/EP2021/052568, filed 3 Feb. 2021, which claims priority to Great Britain Application No. GB2001491.6, filed 4 Feb. 2020, each of which is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, is named 4553.016US1_Sequence_Listing.txt and is 56 kilobytes in size.

### FIELD OF THE INVENTION

The invention relates to methods for screening for an inhibitor of association between candidate binding partners, such as for screening for antagonists of amyloid peptides. The methods employ hybrid proteins each comprising a candidate binding partner and a component of a DNA binding protein, which associate to form a DNA-binding complex capable of inhibiting expression of a reporter. A test compound which inhibits association of the hybrid proteins via their candidate binding partners provides an increase in reporter expression.

### BACKGROUND

Underlying many neurodegenerative diseases is a common mechanism of protein aggregation and subsequent inclusion body formation which are categorised by the major protein involved (Masters et al. 2011). The abnormal aggregation of alpha-synuclein (αS) is associated with neurodegenerative diseases such as Dementia with Lewy bodies, Multiple System Atrophy, and Parkinson's Disease (PD) and amyloid beta (Aβ) aggregation is associated with Alzheimer's Disease (AD). Aggregation begins with the formation of a dimer, which can serve as a template to recruit more monomers, leading to a range of oligomers, and oligomer conformers that are often difficult to target. To date, there has been a lack of success in identifying therapeutics that target and break down protein aggregation.

An alternative therapeutic approach has been proposed that aims to disrupt early stage oligomer formation. However, some of the proteins involved in aggregation-associated disorders are natively disordered, which poses a challenge to traditional drug design. Instead, a number of screening methods have been designed that seek to identify inhibitors from a library of candidate compounds that are able to disrupt oligomer formation. These includes methods that make use of reconstituted split reporter molecules or proteins on aggregation-prone peptides which upon oligomerisation are brought together to create a fluorescent signal (Kurnik et al. 2018), abolish a fluorescent signal (Kim et al. 2006) or generate cell survival (Cheruvara et al. 2015). These signals can be perturbed by an aggregation inhibitor and allow for the identification of potential therapeutic agents.

The screen by Cheruvara et al. used an intracellular protein-fragment complementation assay (PCA) to screen a semi-rational peptide inhibitor library based on the aS fragment 45-54. This fragment was chosen as this is where most early-onset aS point mutations occur. This exploits a split reporter protein of murine dihydrofolate reductase (mDHFR), with one fragment attached to full length WT aS and the other fragment onto members of the peptide library. A successful peptide hit brings together mDHFR, renders it active and as an essential protein, allows cell survival. The initial hits were subjected to multiple passages for competitive growth which identified the strongest inhibitor, peptide fragment 45-54W. Peptide drugs bind targets with high specificity and this intracellular assay, in BL21 Escherichia coli (E. coli), allows for the selection of target-specific peptides that are also soluble, resistant to bacterial proteases, are non-toxic, and that should also function to populate the target in a non-toxic state to be selected. However, it is not clear which aS oligomer or conformation is targeted or the mechanism of inhibition.

The study by Kurnik et al. used two populations of aS labelled either with Tb³⁺ or fluorescein which on aS aggregation gave a fluorescent signal. This in vitro high-throughput method can be conducted in a plate reader and was used to screen 746,000 small compounds. Initial hits which reduced the fluorescence signal by over 50% were further tested to give 9 potential leads that inhibited aS aggregation and reduced the ability of aS oligomers to permeabilise membranes. The ability to derive reproducible and physiologically relevant methods to induce aggregation of aS in vitro is a valuable tool to test a large number of inhibitors in a high-throughput manner. Although hits were screened in vitro, the final 9 inhibitor leads were tested on OLN93 oligodendrocyte cells, a neuronal cell line, for ability to reduce cytotoxicity caused by aS oligomers applied in the cell media, identifying a final 6 compounds with therapeutic potential.

The study by Kim et al. used GFP fused Abeta42 as a method to monitor misfolding. In the absence of inhibition, misfolding and aggregation of Abeta42 caused the entire fusion protein to misfold, thereby preventing fluorescence. In contrast, compounds that inhibited Abeta42 aggregation enabled GFP to fold into its native structure and be identified by the resulting fluorescent signal. However, it is unclear if Abeta42 misfolding ensures loss of GFP folding and therefore if GFP remains fluorescent in the presence of low-n oligomers, and again unclear which oligomer or conformation is targeted or the mechanism of inhibition.

Thus, there remains a need for methods that are able to identify potential inhibitors of dimerization between two proteins, for example proteins that are associated with the formation of aggregation involved in neurodegenerative diseases.

The present invention has been devised in light of the above considerations.

### DISCLOSURE OF THE INVENTION

The screening method of the present invention makes use of a reporter expression cassette that encodes a reporter expression product, such as a protein that provides a phenotypic readout (also termed a “reporter protein”). The reporter expression cassette contains a binding site for a DNA-binding complex. Binding of the DNA-binding complex to the binding site inhibits transcription of the reporter expression cassette, thereby inhibiting expression of the reporter expression product.

This system is employed to investigate association between candidate binding partners (e.g. protein-protein interactions), by linking those candidate binding partners to components of a DNA-binding protein that must be in functional proximity in order to bind DNA. Each candidate binding partner is linked to a respective component of the DNA-binding protein, forming a “first hybrid protein” and a “second hybrid protein”. If the first and second candidate binding partners associate, this will bring the first and second components of the DNA-binding protein into functional proximity, enabling the resulting complex to bind the DNA-binding site within the reporter expression cassette and inhibit expression of the reporter expression product. If a test compound is able to inhibit association of the first and second candidate binding partners, this will inhibit formation of the complex between the first and second hybrid proteins and, in turn, inhibit DNA binding.

If the test compound is able to inhibit association between the first and second candidate binding partners, the expression level of the reporter expression product will be higher in the presence of the test compound than in the absence of the test compound, i.e. there will be an increase in expression of the reporter expression product in the presence of the test compound.

Aggregation involves multiple steps, starting with initial nucleation, oligomer growth, structural interconversion of oligomers and fibril formation (Arosio et al. 2015). The first species in the formation of amyloid is a transiently populated dimer. Once formed, this species can either dissociate back to monomers, or seed the assembly of kinetically trapped higher-n oligomers and their polymorphs. As described above, there has been a lack of success in developing therapeutics that target and either prevent or break down protein aggregation. Moreover, many proteins associated with aggregation, such as the amyloid sequences, are intrinsically disordered in the monomeric state making them difficult to target by rational design based approaches.

The approach described herein, that aims to identify compounds that inhibit association between binding partners is therefore particularly suited for identifying potential therapeutics of diseases associated with protein aggregation. Importantly, the inhibitors identified using the methods described herein are useful for identifying agents capable of inhibiting initial dimer formation, thereby targeting the first event in the formation of aggregates, e.g. by binding to the corresponding monomers.

Blocking the earliest, most upstream point from the formation of complex oligomeric distributions (i.e. by binding to the monomeric protein) is advantageous because the target is more tractable; directly inhibiting production of toxic downstream events. Furthermore, many oligomers and conformers of oligomers can be toxic, and the present methods may simplify the search for potential therapeutics by identifying inhibitors of the earliest stage of oligomerisation or aggregation, i.e. the monomer, or the formation of the dimer. Inhibitors that target only higher-n oligomers will typically not be selected since they will not prevent DNA binding. The approach described herein contrasts favourably to screening assays such as those described in Cheruvara et al., 2015, which aims to identify inhibitors of aggregation but does not distinguish between those inhibitors that target the initial dimerization event and those bind and act on higher-n oligomers.

Whilst the particular screening methods exemplified herein are demonstrated in the context of identifying inhibitors of amyloid proteins, it will also be appreciated that this technique is not limited to the use of amyloids. Rather, the technique can be used to identify compounds that are able to inhibit association at any protein interaction interface, such as within protein complexes.

A further advantage of the methods described herein is that a positive result (i.e. a finding that a test compound does inhibit interaction between the binding partners) is indicated by an increase in reporter expression. Methods in which an increase in expression indicates a positive result are typically less prone to false positives than methods which rely on detecting a decrease in expression. The screening methods described herein therefore produce results with a high degree of confidence, reducing the need for additional screening to confirm the result.

Thus, in one aspect the present invention provides a method for screening for an inhibitor of association between first and second candidate binding partners, the method comprising:

providing a cell, wherein the cell comprises:

a test compound;

a first hybrid protein comprising a first component of a DNA-binding protein linked to a first candidate binding partner;

a second hybrid protein comprising a second component of the DNA-binding protein linked to a second candidate binding partner; and

a reporter expression cassette that encodes a reporter expression product,

wherein the first and second hybrid proteins form a complex having DNA-binding activity upon association of the first and second candidate binding partners, and wherein the reporter expression cassette comprises at least one binding site for the DNA-binding protein such that binding of the complex to the binding site inhibits expression of the reporter expression product; and

determining expression of the reporter expression product;

wherein an increase in expression of the reporter expression product in the presence of the test compound indicates that the test compound is capable of inhibiting association between the first and second candidate binding partners.

By using living cells, the methods described in this aspect have the added benefit of avoiding selection of test compounds that are toxic, susceptible to proteases, insoluble, or non-specific for candidate binding partners and detrimental to cell growth. This method can therefore advantageously be used to select for inhibitors that bind to the candidate binding partner, inhibit dimerization and lack cell toxicity in a single step.

In another aspect, the present invention provides a method for screening for an inhibitor of association between first and second candidate binding partners, the method comprising:

providing a cell-free expression system comprising:

a test compound

a first hybrid protein comprising a first component of a DNA-binding protein linked to a first candidate binding partner;

a second hybrid protein comprising a second component of the DNA-binding protein linked to a second candidate binding partner; and

a reporter expression cassette that encodes a reporter expression product,

wherein the first and second hybrid proteins form a complex having DNA-binding activity upon association of the first and second candidate binding partners,

and wherein the reporter expression cassette comprises at least one binding site for the DNA-binding protein such that binding of the complex to the binding site inhibits expression of the reporter expression product; and

determining expression of the reporter expression product;

wherein an increase in expression of the reporter expression product in the presence of the test compound indicates that the test compound is capable of inhibiting association between the first and second candidate binding partners.

The reporter expression product may be referred to as simply the “reporter”, and its expression as “reporter expression”.

The methods of the invention may comprise comparing reporter expression in the presence of the test compound with a reference level of reporter expression. The reference level of reporter expression may be determined, for example, in the absence of test compound, or in the presence of a reference compound (e.g. a control compound). The reference (or control) compound may be a compound which is known not to inhibit complex formation between the hybrid proteins, i.e. a negative control compound.

The reference level of reporter expression may be determined in the same cell or expression system. For example, reporter expression level may be determined in the same cell or expression system before and after contacting the cell or expression system with the test compound.

Alternatively, the reference level of reporter expression may be determined in a reference cell or expression system, comprising the hybrid proteins and reporter expression cassette, but not comprising the test compound. The reference cell or expression system may comprise a reference (or control) compound instead of the test compound. The test cell or expression system and the reference cell or expression system may be otherwise identical, and may be tested under otherwise identical conditions.

An increase in reporter expression in the presence of the test compound typically indicates that the test compound is capable of inhibiting association between the first and second binding partners.

The methods of the invention may comprise the step of contacting the cell or expression system with the relevant test compound. Alternatively, when the test compound is a peptidic compound, the test compound may be expressed within the cell or expression system.

The first and second candidate binding partners may be amyloid peptides. In certain embodiments, the first and second candidate binding partners are amyloid β (Aβ) peptides, for example those having an amino acid sequence of SEQ ID NO: 49. In other embodiments, the first and second candidate binding partners are α-synuclein (αS) polypeptides, for example those having an amino acid sequence of SEQ ID NO: 53.

An aspect of the present invention relates to a fusion protein comprising a component of a DNA-binding protein and an amyloid peptide component capable of dimerization;

wherein said fusion protein forms a complex capable of binding DNA upon dimerization via the amyloid peptide component.

In some embodiments, the amyloid peptide components are amyloid-β (Aβ) peptides or are α-synuclein (αS) polypeptides.

Another aspect of the present invention relates to inhibitors identified by the methods of the present invention.

The invention also provides cells, libraries and kits as further defined herein.

Some particular aspects of the invention will now be discussed in more detail.

Reporter Expression Product

The reporter expression product used herein can be a peptidic compound or an RNA molecule (such as microRNA, siRNA, or a ribozyme). Methods of measuring expression of protein are well known in the art and include western blot, immunohistochemistry, luciferase gene reporter assays, colorimetric assays such as the BCA assay or Bradford assay, UV spectroscopy, as well as methods that involve observing the phenotypic readout of the protein, as described in more detail below. Methods of measuring the expression of an RNA molecule are also well known in the art and include quantitative PCR (qPCR), transcriptomic analyses, UV spectroscopy and microfluidic analysis. Preferably, the expression product is a protein.

In preferred embodiments, the reporter expression product is a protein that provides a phenotypic readout (also termed a “reporter protein”). A reporter protein that provides a phenotypic readout advantageously allows for a simple and rapid screening of test compounds.

Examples of reporter proteins include cell survival proteins, cell reproduction proteins, fluorescence proteins, bioluminescence proteins, enzymes that act on a substrate to produce a colorimetric signal, protein kinases, proteases, transcription factors, and regulatory proteins such as ubiquitin. The use of suitable reporter proteins in assays for determining PPIs is described, for example, in Wehr and Rossner (2016).

In some embodiments, the reporter protein is a cell survival protein or a cell reproduction protein. A cell survival protein is a protein that is essential for cell survival, such that survival is dependent upon the presence or activity of the cell survival protein. A cell reproduction protein is a protein that is essential for reproduction of the cell, such that cell proliferation (division) is dependent upon the activity of the cell reproduction protein. The essentiality of the cell survival or cell reproduction protein may depend on certain conditions, e.g. the presence of certain factors, such as a cytotoxic compound, in the cell medium.

If the reporter protein is a cell survival protein, then inhibition of expression of the cell survival protein will result in cell death. Thus, in methods described herein where the reporter protein is a cell survival protein, binding of the DNA-binding protein to the binding site in the absence of a test compound will result in cell death. Cell death can be determined by one of a number of techniques known to the person skilled in the art, e.g. the observing of morphological changes such as cytoplasmic blebbing, cell shrinkage, internucleosomal fragmentation and chromatin condensation. DNA cleavage typical of the apoptotic process may be demonstrated using TUNEL and DNA ladder assays. In these situations, when a test compound is added that is able to inhibit association between first and second candidate binding partners, this will result in cell survival and therefore such a method uses cell survival as an indicator that the test compound is an inhibitor of dimerization. Use of a cell survival protein as a reporter protein can be advantageous as it gives a simple binary readout, i.e. the cell is either dead or alive.

If the reporter protein is a cell reproduction protein, then inhibition of expression of the cell reproduction protein will result in the cell being unable to proliferate and therefore unable to form progeny. Thus, in methods described herein where the reporter protein is a cell reproduction protein, binding of the DNA-binding protein to the binding site in the absence of a test compound will inhibit cell proliferation. Cell proliferation can be determined by one of a number of techniques known to the person skilled in the art, e.g. by counting of individual cells, foci or colonies, measuring metabolic activity using dyes such as MTT and WST-1, using nucleoside analogues such as bromodeoxyuridine (BrdU) and measuring incorporation of this analogue in the cells, staining dividing cells using reagents such as succinimidyl ester of carboxyfluorescein diacetate, and detecting proliferation markers such as PCNA, poisomerase IIB or phosphohistone H3. Inhibition of cell proliferation may also result in cell death, which can be measured as described above. In these situations, when a test compound is added that is able to inhibit association between first and second candidate binding partners, this will restore cell proliferation and therefore such a method uses cell proliferation as an indicator that the test compound is an inhibitor of dimerization.

Examples of cell survival proteins include enzymes that are involved in synthesising compounds that are required for cell survival and proteins that are capable of inhibiting action of a toxic agent, such as an antibiotic. Examples of cell reproduction proteins include enzymes that are required for cell reproduction.

Examples of enzymes that are involved in synthesising compounds required for cell survival or reproduction are set out in Table 1. Thus, in some embodiments, the cell survival protein or cell reproduction protein is an enzyme selected from the first column Table 1.

TABLE 1
Example enzymes involved in synthesising compounds required for cell survival or reproduction

| Enzyme | Compounds/conditions able to inhibit enzyme function |
|---|---|
| Dihydrofolate reductase (DHFR) | methotrexate or trimethoprim, cultured |
| | without nucleosides |
| Thymidine kinase | ganciclovir, hypoxanthine/aminopterin/thymidine (HAT) |
| thymidylate synthase | 2 fluorodeoxyuridine |
| Xanthine-guanine phosphoribosyl | mycophenolic acid with limiting xanthine |
| Asparagine synthetase | B-aspartyl hydroxamate or albizin puromycin |
| Cytosine methyltransferase | 5-Azacytidine (5-aza-CR) and 5-aza-2′-deoxycytidine |
| O6-alkylguanine alkyltransferase | N-methyl-N-nitro-sourea |
| Glycinamide ribonucleotide transformylase | dideazatetrahydrofolate, cultured without purine |
| Glycinamide ribonucleotide synthetase | cultured without purine |
| Phosphoribosyl-aminoimidazole synthetase | cultured without purine |
| Formylglycinamide ribotide amidotransferase | L-azaserine, 6-diazo-5-oxo-L-nor-leucine, |
| | cultured without purine |
| Phosphoribosyl-aminoimidazole carboxylase | cultured without purine |
| Phosphoribosyl-aminoimidazole | cultured without purine |
| carboxamide formyltransferase | |
| Fatty acid synthase | cerulenin |
| IMP dehydrogenase | mycophenolic acid |
| histidinol dehydrogenase | cultured without histidine |

For example, dihydrofolate reductase (DHFR) catalyses the reduction of dihydrofolate to tetrahydrofolate, for use in transfer of one-carbon units required for biosynthesis of serine, methionine, purines, pantothenate and thymidylate. In the absence of DHFR function, de novo synthesis of nucleoside precursors (hypoxanthine and thymidine) is inhibited. Thus, if cells are grown in the absence of a functioning DHFR and in the absence of nucleosides (e.g. all nucleosides, or at least the purine nucleosides), the cells will die. Reconstitution of enzyme activity can be monitored in vivo by cell survival in DHFR-negative cells grown in the absence of nucleosides.

Examples of proteins that are capable of inhibiting action of a toxic agent include enzymes that are capable of metabolising a toxic agent, e.g. to a less toxic agent, and antibiotic resistance proteins, e.g. proteins that bind and inhibit antibiotics. Examples of these are set out in Table 2. Thus, in some embodiments, the cell survival protein is a protein selected from the first column in Table 2.

TABLE 2
Examples of proteins that are capable
of inhibiting action of a toxic agent

| Cell survival protein | Toxic agent/antibiotic |
|---|---|
| beta-lactamase | β-lactam antibiotics such as |
| | penicillins, cephalosporins, cephamycins |
| chloramphenicol | chloramphenicol |
| acetyl transferase | |
| Puromycin | puromycin |
| N-acetyltransferase | |
| Aminoglycoside | neomycin, G418, gentamycin |
| phosphotransferase | |
| Hygromycin B | hygromycin B |
| phosphotransferase | |
| Blebomycin binding | Blebomycin |
| protein | |
| Adenosine deaminase | Xyl-A or adenosine, alanosine, |
| | and 2′-deoxycoformycin |

For example, Hygromycin-B is an aminocyclitol that inhibits protein synthesis by disrupting translocation and promoting misreading. The E. coli enzyme hygromycin-B-phosphotransferase detoxifies the cells by phosphorylating hygromycin-B. When expressed in mammalian cells, hygromycin-B-phosphotransferase can confer resistance to hygromycin-B (Gritz and Davies, 1983).

As a further example, adenosine deaminase (ADA) catalyses the irreversible conversion of cytotoxic adenine nucleosides to their respective conversion of cytotoxic adenine nucleosides to their respective nontoxic inosine analogues. ADA only becomes a cell survival protein when cytotoxic concentrations of adenosine are added. By adding cytotoxic concentrations of adenosine or cytotoxic adenosine analogues such as 9-b-D-xylofuranosyladenine to the cells, ADA is required for cell growth to detoxify the cytotoxic agent. An exemplary method that uses ADA as a reporter protein is described in Kaufman et al. 1986.

Bleomycin, a member of the leomycin/phyleomycin family of antibiotics, is toxic to bacteria, fungi, plants, and mammalian cells. The expression of the bleomycin binding protein confers resistance by binding to and sequestering the drug and thus preventing its association and hydrolysis of DNA.

Methods using cell survival proteins as reporter proteins in screening for inhibitors that disrupt PPIs are known. See, for example, Park et al. (2007), which describes methods involving beta-lactamase in a fragmentation complementation strategy.

In some embodiments, the cell survival protein is a dihydrofolate reductase (DHFR). The DHFR may be murine DHFR, which may be the protein identified by UniProt accession number P00375-1 (version 3, last modified 23 Jan. 2007). For example, the murine DHFR may have an amino acid sequence that is at least 80%, at least 85%, or at least 90% identical to the sequence set forth in SEQ ID NO: 1. In particularly preferred embodiments, the murine DHFR has an amino acid sequence that is at least 90%, at least 91%, at least 92%, at least 93%, at least 94%, at least 95%, at least 96%, at least 97%, at least 98%, at least 99%, or 100% identical to the sequence set forth in SEQ ID NO: 2.

The DHFR may be human DHFR, which may be the protein identified by UniProt accession number P00374-1 (version 2, last modified 23 Jan. 2007). For example, the human DHFR may have an amino acid sequence that is at least 80%, at least 85%, or at least 90% identical to the sequence set forth in SEQ ID NO: 3.

In some embodiments, the cell may be conditionally dependent upon the activity of the cell survival protein or cell reproduction protein for its survival or reproduction, respectively. In some cases, the cell may not contain an endogenous cell survival protein or cell reproduction protein and therefore requires the addition of an exogenous protein to proliferate. In other cases, the cell may contain an endogenous cell survival protein or endogenous cell reproduction protein that is necessary for cell survival or proliferation, respectively, and the function of this endogenous protein can be inhibited or removed in certain conditions (also termed “selection conditions”). Thus, the cell can make use of the endogenous protein for its survival until the selection conditions are activated, at which point the activity of the cell survival protein (also termed an “exogenous cell survival protein”) becomes essential for the cell's survival. This is advantageous as it allows the cells to survive until the screening method is ready to be run. It may be possible to elicit these selection conditions using, for example, a selection agent, where the selection agent is a compound that inhibits the activity of endogenous protein but does not inhibit the activity of the cell survival protein. For example, the endogenous cell survival protein or cell reproduction protein may one of the proteins set out in the first column Table 1, above and may be inhibited using the selection conditions set out in the second column.

The term “endogenous” in the context of cell survival proteins and cell reproduction proteins is intended to mean a protein that originates from the cell in which the screening method is being performed. The term “exogenous” in the context of cell survival proteins and cell reproduction proteins is intended to mean a protein that has equivalent activity to the endogenous protein such that it can compensate for a deficiency in the function of the endogenous cell survival protein, but is resistant to selection conditions, e.g. a the presence of a particular compound, that inhibit the function of the endogenous protein, such that survival or proliferation of the cell is dependent upon the activity of the exogenous protein under these selection conditions. The exogenous and endogenous protein will normally have similar, but not identical amino acid sequences. For example, the exogenous protein may be at least 80%, at least 85%, at least 90%, or at least 95% identical to the endogenous protein and the exogenous protein may contain one or more modifications in its amino acid sequence compared to the amino acid sequence of the endogenous cell survival protein. The exogenous protein and endogenous protein may be orthologues, i.e. genes from different species that descended from a common ancestral sequence. For example, the endogenous cell survival protein or cell reproduction protein may be a bacterial version of the cell survival protein or cell reproduction protein set out above, e.g. in Table 1 or Table 2, and the exogenous protein may be an orthologous protein from a mammalian species, e.g. murine or human. Alternatively or additionally, the exogenous protein may contain one or more mutations in its amino acid sequence that render it resistant to the selection conditions that inhibits the function of the endogenous protein.

For example, where the cell is a bacterial cell, the endogenous protein may be a bacterial cell survival protein and the exogenous cell survival protein may be an orthologous eukaryotic cell survival protein, such as a mammalian cell survival protein, e.g. mouse or human cell survival protein. A bacterial specific inhibitor can then be used as the selection agent to inhibit the bacterial cell survival protein without affecting the function of the eukaryotic cell survival protein.

In a more specific example, the bacterial cell survival protein may be DHFR from E. coli, which may be the protein identified by UniProt accession number POABQ4-1 (version 1, last modified 21 Jul. 1986) and the eukaryotic cell survival protein may be mouse or human DHFR, as set out above. Bacterial DHFR, can be specifically inhibited using compounds such as trimethoprim, rendering cells dependent upon the activity of exogenous DHFR, e.g. murine or human DHFR, for their survival.

Thus, the bacterial cells may be grown in a medium, such as a rich liquid broth medium, until the screening method is ready to be performed. At this point the cells can make use of the endogenous protein in order to survive and/or proliferate. When the screening method is ready to be performed, the cells may be grown in a medium that lacks nucleosides such a purines and a selection agent, such as trimethoprim that inhibits bacterial DHFR, added. Once the selection agent is added, the cells are conditionally dependent on the activity of the exogenous cell survival protein, such as mammalian or murine DHFR, for its survival. Cell survival will therefore be dependent on the activity of the cell reporter protein and an increase in cell survival will indicate that the test compound is capable of inhibiting association between first and second candidate binding partners. A person of ordinary skill in the art would be able to select an appropriate type and amount of selection agent to use such that cell survival is dependent on the activity of the cell reporter protein. For example, where TMP is used to inhibit bacterial DHFR, the concentration may be between 4-20 μM.

In another example, where the cell is a mammalian cell, the endogenous cell survival protein may be a mammalian DHFR. Methods of using detecting PPIs using a mammalian DHFR as a cell survival protein are described in Remy et al. (2007). Briefly, the principle of the DHFR survival assay in mammalian cells is that cells lacking endogenous DHFR activity, can be rescued by the simultaneous expression of complementary DHFR in media depleted of nucleosides. The assay could be performed in DHFR-negative cells, or selection can be achieved in DHFR-positive cells using an exogenous DHFR as the cell survival protein, where the exogenous DHFR contains one or more mutations that render the DHFR resistant to a selection agent, such as the anti-folate drug methotrexate (MTX). When the cells are grown in the absence of nucleotides with selection for MTX resistance, only those cells that can make use of the exogenous DHFR will survive.

An example of a mutation in a mammalian DHFR that renders the mammalian DHFR resistant to MTX is the F31S mutation, wherein residue numbering is according to the murine DHFR set forth in SEQ ID NO: 1. Thus, the cell survival protein may be a murine DHFR that has an amino acid sequence that is at least 80%, at least 85%, at least 90%, or at least 95% identical to the sequence set forth in SEQ ID NO: 1, wherein the murine DHFR further comprises a serine (S) at position 31, and wherein residue numbering is according to the murine DHFR set forth in SEQ ID NO: 1. The cell survival protein may be a murine DHFR that has an amino acid sequence that is at least 90%, at least 91%, at least 92%, at least 93%, at least 94%, at least 95%, at least 96%, at least 97%, at least 98%, at least 99%, or 100% identical to the sequence set forth in SEQ ID NO: 2, wherein the murine DHFR further comprises a serine (S) at position 31.

Such a method may comprise growing the mammalian cells comprising the exogenous cell survival protein under conditions where the cell is dependent on the activity of the exogenous cell reporter protein for survival. For example, the exogenous cell survival protein may be murine DHFR that has been modified to be resistant to the anti-folate drug methotrexate (MTX), and the mammalian cell may be grown in the absence of nucleosides and in the presence of MTX. Cell survival will therefore be dependent on the activity of the cell reporter protein and an increase in cell survival will indicate that the test compound is capable of inhibiting association between the first and second candidate binding partners.

As a further example of a reporter protein that provides an observable phenotype, the reporter protein can be a fluorescent reporter protein. In these cases, binding of the DNA-binding protein to the binding site in the absence of a test compound will inhibit the fluorescent signal. When a test compound is added that is capable of inhibiting association between the first and second candidate binding partners, this will result in an increase in fluorescent signal and therefore such a method uses fluorescence as an indicator that the test compound is an inhibitor of dimerization. The cells expressing fluorescence could be sorted (e.g. by fluorescence-activated cell sorting, FACS) in order to rank cells by fluorescence and therefore the most effective test compound(s), where the cell with the highest level of fluorescence indicates the most effective test compound.

Thus, in some embodiments, the reporter protein is a fluorescent reporter protein, such as green fluorescent protein (GFP), yellow fluorescent protein (YFP), mNeonGreen, mCherry or Kusabira-Green fluorescent protein (mKG).

In some embodiments, the reporter protein is a bioluminescence protein, such as a luciferase enzyme. Such proteins work in a similar manner to fluorescent proteins, except that instead of requiring an external light source, they require the addition of luciferin. Cells expressing bioluminescence can be sorted, e.g. by FACS, to rank cells in a similar manner as described above for fluorescence.

In another example of a reporter protein that provides an observable phenotype, the reporter protein can be an enzyme that acts on a substrate to produce a colorimetric signal. In these cases, binding of the DNA-binding protein to the binding site in the absence of a test compound will inhibit the colorimetric signal. When a test compound is added that is a is capable of inhibiting association between the first and second candidate binding partners, this will result in an increase in the colorimetric signal and therefore such a method uses colorimetric signal as an indicator that the test compound is able to inhibit dimerization.

Thus, in some embodiments, the reporter protein is an enzyme that acts on a substrate to produce a colorimetric signal. For example, the enzyme may be horseradish peroxidase or beta-galactosidase.

A further example of a reporter protein is a protein kinase, such as the focal adhesion kinase (FAK). FAK is a tyrosine kinase that is made up of distinct domains that are phosphorylated. Phosphorylation can be detected by, for example, lysing and immunoblotting the cell lysate. A method of probing protein-protein interactions using FAK is described, for example, by Ma et al. (2014).

Thus, in some embodiments, the reporter protein is a protein kinase, such as FAK.

Another example of a reporter protein is a protease, such as tobacco etch virus protease (TEV). TEV is a highly specific viral cysteine protease and can be applied to analyse PIPIs using a modular approach of various reporters, including ‘silent’ fluorescent and luminescent reporter proteins that require proteolysis in order to become active.

Thus, in some embodiments, the reporter protein is a protease, such as TEV, used in combination with a silent fluorescent or luminescent reporter protein that requires proteolysis in order to become active. A method of monitoring PIPIs using TEV is described, for example, by Wehr et al. (2006).

In some embodiments, the reporter protein is a transcription factor, such as a transcriptional activator. Examples of transcriptional activators include GAL4, which is well known for its use in “two hybrid” systems for studying PPIs. See, for example, Young, 1998. A transcriptional activator binds a DNA sequence causing activation of a downstream reporter gene. For example, GAL4 binds the UAS and drives transcription of the downstream reporter gene. The downstream reporter gene may encode any of the reporter proteins described above, for example, it may encode a cell survival protein or may encode a fluorescent protein. Expression of the transcriptional activator can therefore be measured indirectly by measuring expression of the protein encoded by the downstream reporter gene.

In some embodiments, the reporter protein is not a split reporter protein. Split reporter proteins are made up of a functional reporter protein that has been split into two or more inactive fragments, i.e. the inactive fragments do not provide a phenotypic readout unless they are reassembled. Thus, in some embodiments the reporter protein is capable of providing a phenotypic readout without requiring reassembly with another protein or peptide.

Candidate Binding Partners

The candidate binding partners can be any peptidic molecules that associate with one another (or are expected to do so). The first and second binding partners may have an identical amino acid sequence. Alternatively, the first and second binding partners may have different amino acid sequences.

The candidate binding partners may form protein aggregates, or may be expected to do so. Protein aggregates are typically formed where multiple misfolded proteins accumulate and clump together and their presence is associated with a number of diseases, in particular neurodegenerative diseases such as Alzheimer's Disease (AD), Parkinson's disease (PD) and prion disease (also known as transmissible spongiform encephalopathy). In some embodiments, the presence of an aggregate of the candidate binding partners in a human patient is associated with a disease or other pathological condition, such as a neurodegenerative disease.

Examples of peptides and polypeptides that are capable of forming protein aggregates include those that are capable of aggregating to form amyloids, as well as those capable of aggregating to form amorphous or native-like deposits.

The candidate binding partners may be capable of aggregating to form amyloid, i.e. which are capable of aggregating to form a “cross-beta” structure, in vivo or in vitro such molecules may be referred to as amyloid peptides or amyloid proteins. Typically, the candidate binding partners are provided as monomeric peptides, e.g. monomeric amyloid peptides.

Examples of peptides and polypeptides known to form amyloid, and their associated diseases, include:

| Peptide or | | |
|---|---|---|
| polypeptide name | Abbreviation | Disease, e.g. |
| Beta amyloid from Amyloid precursor protein | Aβ from APP | Alzheimer's disease |
| Islet amyloid polypeptide (Amylin) | AIAPP | Diabetes mellitus type 2 |
| Alpha-synuclein | αS | Parkinson's disease and other synucleinopathies |
| Tau protein | Tau | Various tauopathies |
| Prion protein | PrP | Transmissible spongiform encephalopathy |
| | | (e.g. bovine spongiform encephalopathy) |
| Huntingtin | none | Huntington's disease |
| Calcitonin | ACal | Medullary carcinoma of the thyroid |
| Atrial natriuretic factor | AANF | Cardiac arrhythmias, isolated atrial amyloidosis |
| Apolipoprotein AI | AApoA1 | Atherosclerosis |
| Serum amyloid A | SAA | Rheumatoid arthritis |
| Medin | AMed | Aortic medial amyloid |
| Prolactin | APro | Prolactinomas |
| Transthyretin | ATTR | Familial amyloid polyneuropathy |
| Lysozyme | ALys | Hereditary non-neuropathic systemic amyloidosis |
| Beta-2 microglobulin | Aβ2M | Dialysis related amyloidosis |
| Gelsolin | AGel | Finnish amyloidosis |
| Keratoepithelin | AKer | Lattice corneal dystrophy |
| TDP43, FUS, SOD | TDP43, | ALS |
| | FUS, SOD | |
| Cystatin | ACys | Cerebral amyloid angiopathy (Icelandic type) |
| Immunoglobulin light chain AL | AL | Systemic amyloid light-chain (AL) amyloidosis |
| Immunoglobulin heavy chain | AH | Heavy-chain amyloidosis |
| S-IBM | none | Sporadic Inclusion body myositis |
| ABri peptide | ABri | Familial British dementia |
| ADan peptide | ADan | Familial Danish dementia |
| Insulin | none | Injection-localized amyloidosis |
| β2-microglobulin | β2-m | Dialysis-related amyloidosis, and Hereditary |
| | | visceral amyloidosis |
| N-term fragments of apolipoprotein A-I | ApoAI | ApoAI amyloidosis |
| C-term extended apolipoprotein A-II | ApoAII | ApoAII amyloidosis |
| N-term fragments of apolipoprotein A-IV | ApoAIV | ApoAIV amyloidosis |
| Apolipoprotein C-II | ApoCII | ApoCII amyloidosis |
| Apolipoprotein C-III | ApoCIII | ApoCIII amyloidosis |
| Fragments of fibrinogen α-chain | none | Fibrinogen amyloidosis |
| Atrial natriuretic factor | ANF | Atrial amyloidosis |

These and other amyloid peptides and examples of associated diseases are set out in Chiti and Dobson, 2017. See, in particular Table 1 of Chiti and Dobson, 2017. Thus, the candidate binding partners may comprise an amyloid peptide listed above and/or in Table 1 of Chiti and Dobson, 2017.

Examples of peptides and polypeptides capable of forming non-amyloid deposits (such as amorphous deposits), and their associated diseases, include:

| Peptide or | |
|---|---|
| protein name | Disease, e.g. |
| Neurogenic locus notch homolog | Cerebral autosomal dominant arteriopathy with |
| protein 3 (Notch 3) ectodomain | subcortical infarcts and leukoencephalopathy (CADASIL) |
| Immunoglobulin (Ig) heavy chains | Heavy-chain deposition disease |
| | (renal disease) |
| Ig light chains | Light-chain deposition disease, Myeloma cast |
| | nephropathy, and Fanconi syndrome (all are renal) |
| Fibronectin (FN) | FN glomerulopathy |
| TAR DNA-binding protein 43 (TDP-43) | Frontotemporal lobar degeneration with ubiquitin-positive |
| | inclusions, and Amyotrophic lateral sclerosis |
| RNA-binding protein FUS (FUS) | Frontotemporal lobar degeneration with ubiquitin-negative |
| | inclusions, and Amyotrophic lateral sclerosis |
| [Cu—Zn] superoxide | Amyotrophic lateral sclerosis |
| dismutase (SOD1) | |
| Complement C1q | C1q nephropathy |
| subcomponent (C1q) | |
| Immunoglobulin A (IgA) | IgA nephropathy (Berger disease), and Henoch-Sch\ddot{ }onlein |
| | purpura |
| Alanine:glyoxylate | Primary hyperoxaluria type 1 |
| aminotransferase (AGT) | |
| Immunoglobulin M (IgM) | Multiple myeloma/plasmacytoma (Russell bodies) |
| Immunoglobulin G | Multiple myeloma/plasmacytoma (Russell bodies) |
| (IgG) | |
| Uromodulin, or Tamm-Horsfall | Medullary cystic kidney disease 2, Familial juvenile |
| urinary glycoprotein (THP) | hyperuricemic nephropathy, and Glomerulocystic kidney disease |
| Ataxin-1 | Spinocerebellar ataxia 1 |
| Hemoglobin | Sickle cell anemia, Heinz body anemia, |
| | and Inclusion body β-thalassemia |
| α1-Antitrypsin | α1-Antitrypsin deficiency |
| Ferritin light chain | Hereditary hyperferritinemia cataract syndrome |
| Actin | Alzheimer disease, and Frontotemporal dementia |
| Cellular tumor antigen p53 (p53) | Cancer |

These and other non-amyloid peptides and polypeptides and examples of associated diseases are set out in Chiti and Dobson, 2017. See, in particular Table 2 of Chiti and Dobson, 2017. Thus, the candidate binding partners may comprise a peptide or polypeptide capable of forming non-amyloid deposits listed above and/or in Table 2 of Chiti and Dobson, 2017.

Thus the candidate binding partners may be, or may comprise, a peptide having an amino acid sequence from one of these proteins which is capable of dimerization. Typically, the peptide will be capable of aggregation, although not necessarily when linked to a component of a DNA binding protein as described herein.

In some embodiments, the candidate binding partners are amyloid-β (Aβ) peptides, α-synuclein (αS) polypeptides, tau proteins, or prion proteins.

In some embodiments, the candidate binding partners are amyloid-β (Aβ) peptides. Amyloid-β precursor protein (AβPP) is a major transmembrane protein found at neuronal synapses and can be sequentially cleaved by β- and γ-secretases to release amyloid-β (Aβ) peptides into the intercellular space (O'Brien and Wong, 2011; Pospich and Raunser, 2017). Aβ peptides vary in length between 39-42 amino acids, as γ-Secretase cleave several sites in the transmembrane domain of AβPP (Takami et al., 2009; Andrew et al., 2016). 15% of released amyloid peptide are 42 amino acids long (Golde, Eckman and Younkin, 2000) which is the most fibrillogenic form making it the major component found in plaques derived from AD patients (Pospich and Raunser, 2017). Recent cryo-electron microscopy data of a highly homogeneous form of fibrilous Aβ₁₋₄₂ revealed that the intrinsically disordered peptides form ordered helical structures when in fibres (Gremer et al., 2017).

In some embodiments, the candidate binding partners are Aβ peptides having 42 amino acids (Aβ₁₋₄₂), In some embodiments, the first and second candidate binding partners comprise an amino acid sequence having the sequence of SEQ ID NO: 49.

In some embodiments, the candidate binding partners are α-synuclein (αS) polypeptides. αS is a small 14 kDa, 140 amino acid, natively unfolded protein (lacks persistent secondary and tertiary structure) of which little is known about the normal structure and function. However, high expression in pre-synaptic terminals (Jakes et al. 1994), adoption of an α-helical structure on interaction with membranes (Jao et al. 2004; Ulmer et al. 2005) and its promotion of SNARE-complex assembly (Burré et al. 2010) has implicated αS in the regulation of synaptic function and plasticity as well as neurotransmitter release (Lashuel et al. 2013). The native structure of physiological αS has long been controversial, most commonly it is described as monomeric (Burré et al. 2013; Fauvet et al. 2012). The αS polypeptide may be the polypeptide identified by UniProt accession number P37840-1 (version 1, last modified 1 Oct. 1994).

In some embodiments, the first and second candidate binding partners comprise an amino acid sequence having the sequence of SEQ ID NO: 53.

In some embodiments, the candidate binding partners are tau proteins. Tau proteins are proteins that stabilise microtubules and in humans are the product of alternative splicing from the MAPT (microtubule-associated protein tau) gene. Hyperphosphorylation of the tau protein can result in the self-assembly of tangles of paired helical filaments and straight filaments, which are involved in the pathogensis of Alzheimer's disease, frontotemporal dementia and other tauopathies. Tau filaments from the human brain and from in vitro assembly have been demonstrated to show the cross-beta structure associated with amyloids (Berriman et al. 2003). The tau protein may be any one of the isoforms identified by UniProt accession number P10636, i.e. any one of P10636-1, P10636-2, P10636-3, P10636-4, P10636-5, P10636-6, P10636-7, P10636-8, or P10636-9 (version 5, last modified 31 Mary 2011).

In some embodiments, the candidate binding partners are prion proteins. The specific function of prion protein (also known as PrP or CD230) is uncertain, but misfolded versions of PrP isoforms are associated with a variety of cognitive disorders and neurodegenerative diseases. Prion proteins are particularly associated with transmissible spongiform encephalopathies (also known as prion disease), which in humans include Creutzfeldt-Jakob disease (CJD), fatal familial insomnia (FFI), Gerstmann-Sträussler-Scheinker syndrome (GSS), kuru, and variant Creutzfeldt-Jakob disease. Prion proteins form abnormal amyloid aggregates, which accumulate in infected tissue and are associated with tissue damage and cell death. The prion protein may be the polypeptide identified by UniProt accession number P04156-1 (version 1, last modified 1 Nov. 1986).

The candidate binding partners may be known to be associated with post-translational modifications, such as phosphorylation, glycation, nitration or acetylation. The first and candidate binding proteins may be ones where the post-translation modification is associated with aggregation. For example, phosphorylation of the tau protein is associated with its aggregation and the formation of disease-causing filaments.

Thus, in some embodiments the candidate binding proteins comprise one or more post-translational modifications. Where the assay is carried out in a cell, the cell may comprise the necessary components and/or enzymes to post-translationally modify the candidate binding proteins. In such cases, the cell is typically a eukaryotic cell because some prokaryotic cells do not allow for the same post-translational modifications as eukaryotes.

Components of DNA-Binding Protein

The first and second hybrid proteins each comprise a component of a DNA-binding protein, respectively termed a “first component” and a “second component”.

The first and second components are not able to associate with one another directly, but rely on interaction between their respective binding partners in order to associate.

Further, the first and second components are not able to bind DNA individually, or have only minimal DNA-binding activity individually. When the separated components are brought into close proximity (referred to as “functional proximity”) as part of a complex between the hybrid proteins (mediated by interaction between the candidate binding partners), they are able to bind DNA.

Typically, the first and second components are components of the same native DNA-binding protein, e.g. the same transcription factor.

The first and second components may have an identical amino acid sequence, i.e. the DNA-binding complex binds DNA via a homodimer of the same DNA-binding component sequence. Alternatively, the first and second components may have different amino acid sequences, i.e. the DNA-binding complex binds DNA via a heterodimer of the first and second components.

The DNA-binding complex formed in the methods of the invention may bind to a binding site (also known as a recognition site) in a sequence specific manner. Typically, the complex will bind to a recognition site bound by the DNA-binding protein from which the first and second components are derived. For example, the DNA-binding components may be derived from a transcription factor, e.g. a DNA-binding fragment of a transcription factor, e.g. a eukaryotic transcription factor, such as a human transcription factor. The DNA-binding components may be derived from any of the human transcription factors described in Vaquerizas et al. (2009) (e.g. any of those listed in Supplementary information S3). Exemplary transcription factors from which the DNA-binding protein can be derived are set forth below.

The complex is typically not able to activate transcription of the reporter expression product. Thus, the complex lacks a functional domain for activating transcription of the reporter expression product. In some embodiments, the DNA-binding protein lacks transcriptional activation activity. For example, where the first and second components of the DNA-binding protein are derived from a transcription factor, the hybrid proteins lack functional domain(s) of the transcription factor responsible for transcriptional activation (or transcriptional repression). By “lack a functional domain” or “lacks functional domain(s)” it is intended to encompass situations where the domain itself is absent, but also situations where a structurally similar domain is present, but lacks functional activity (e.g. contain amino acid mutations that result in a loss of functional activity).

The disclosed screening method relies on association (e.g. dimerization) of the first and second candidate binding partners to bring the separated components of the DNA-binding protein into close proximity to form the DNA-binding complex and bind DNA. As noted above, the first and second components of the DNA-binding protein should not be able to bind DNA individually, and are not able to bind DNA in combination without both respective binding partners also being present to mediate their association. Thus, the first and second components of the DNA-binding protein should display minimal or no ability to bind DNA if either component is not linked to their respective candidate binding partner. In this situation, minimal ability to bind DNA is intended to mean the first and second components of the DNA-binding protein display less than 10%, preferably less than 5%, more preferably less than 1%, of the DNA-binding activity than is exhibited when the components are expressed as part of the first and second hybrid proteins. Any suitable method can be used to measure DNA-binding activity. For example DNA-binding activity can measured using the TBS assay described in the Examples, where DNA-binding activity results in cell death under selective conditions.

In embodiments where the DNA-binding protein is derived from a transcription factor, preferably the first and second components of the DNA-binding protein lacks functional domain(s) of the transcription factor responsible for association (e.g. dimerization). For example, the first and second components of the DNA-binding protein may lack the dimerization domain found in the native transcription factor. In particularly preferred embodiments where the DNA-binding protein is derived from a transcription factor, the first and second components of the DNA-binding protein lack functional domain(s) of the transcription factor responsible for transcriptional activation or transcription repression and further lack functional domain(s) of the transcription factor responsible for association (e.g. dimerization).

In some embodiments, the first and second components of the DNA-binding protein are DNA-binding fragments of a basic leucine zipper (bZip), basic helix-loop helix (bHLH) or bHLH leucine zipper (bHLH-Zip) transcription factor. bHLH and bHLH-Zip transcription factors are exclusively eukaryotic proteins that bind to sequence-specific double-stranded DNA as homodimers or heterodimers to either activate or repress gene transcription. bZIP transcription factors form one of the largest families of transcription factors in eukaryotic cells and contain a basis region that contacts DNA bases in order to bind to its DNA-binding site. As well as human proteins, certain viral proteins such as BZLF1 form part of the bZIP family. Details of bZIP, bHLH and bHLH-Zip transcription factors and their consensus sequences are provided in Vinson et al. (2002), Newman & Keating (2003) and Rodriguez-Martinez et al. (2017). Typically, the components will be (or will comprise) the basic portions which physically interact with DNA and will lack the portions (e.g. coiled-coil portions) responsible for dimerization.

Exemplary bHLH transcription factors include ATOH1, AhR, AHRR, ARNT, ASCL1, BHLH2, BHLH3, BHLH9, ARNTL, ARNTL2, CLOCK, EPAS1, FIGLA, HAND1, HAND2, HESS, HES6, HEY1, HEY2, HEYL, HES1, HIF1A, HIF3A, ID1, ID2, ID3, ID4, LYL1, MESP2, MXD4, MYCL1, MYCN, MyoD, Myogenin, MYFS, MYF6, Neurogenin1, Neurogenin2, Neurogenin3, NeuroD1, NeuoD2, NPAS1, NPAS2, NPAS3, OLIG1, OLIG2, Pho4, Scleraxis, SIM1, SIM2, TAL1, TAL2, Twist and USF1. Exemplary bHLH-ZIP transcription factors include AP-4, Max, MXD1, MXD3, MITF, MNT, MLX, MLXIPL, MXI1, Myc, SREBP1 and SREBP2. In particular embodiments, the bHLH-ZIP transcription factor used may be c-Myc or Max, or a heterodimer between c-Myc and Max (c-Myc-Max). bHLH and bHLH-ZIP transcription factors typically bind to a consensus sequence called an E-box, which can have the sequence CANNTG (‘N’ being any nucleotide) and in particular cases has the sequence CACGTG. The components of the DNA-binding protein are DNA-binding may be or may comprise the DNA-binding fragments of any of these bHLH or bHLH-Zip transcription factors and the reporter expression cassette comprise at least one E-box as a binding site, where the E-box may have the sequence CANNTG, e.g. CACGTG.

Exemplary human bZIP transcription factor subfamilies, the nucleotide sequences of their binding sites and examples of proteins of these subfamilies are set forth in the following table. The components of the DNA-binding protein may be derived from any of these human bZIP proteins and the reporter expression cassette comprise at least one of these binding sites. For example, the first and second components of the DNA-binding protein may be DNA-binding fragments of a bZIP protein of the Fos/Jun bZip family (e.g. a DNA-binding fragment of cJun) and the at least one binding site may have the nucleotide sequence TGACTCA or TGAGTCA.

| Human bZIP | | Nucleotide sequence(s) | |
|---|---|---|---|
| subfamily | Exemplary bZIP protein | of binding site | Name of binding site |
| PAP | PAP1, YAP1, YAP2, YAP3, | TTACGTAA | PAP/CREB-2/PAR |
| | YAP4, YAP5, YAP6, YAP7, | | |
| | Cap1 | | |
| CREB-2 | AFT4, mATFP4, ApCREB-2, | | |
| | hCREB2, acr1 | | |
| PAR | DBP, VBP/TEF, HLF, CES2, | | |
| | TEF | | |
| C/EBP | C/EBPα, C/EBPβ, C/EBPδ, | ATTGCGCAAT | CCAAT |
| | C/EBPϵ, C/EBPγ, CRP1, | | |
| | CRP2, CRP3, Ig/EBP, lap, | | |
| | DDIT3 | | |
| Fos/Jun | cFos, FRA1 (FosL1), FRA2 | TGACTCA or TGAGTCA | TPA response |
| | (FosL2), eJun, JUNB, JUND, | | element (TRE) |
| | GCN4, BATF, BATF2, | | |
| | BATF3 | | |
| CREB | CREB1, ATF1, ATF2, ATF3, | TGACGTCA | cAMP response |
| | ARF5, ATFa, BBF-2, | | element (CRE) |
| | CREB3L1 | | |
| Maf | MafA, MafB, BACH1, BACH2 | TGCTGA(G/C)TCAGCA and | Mat recognition |
| | | TGCTGAG(C/C)GTCAGCA | element (MARE) |

AP-1 is a dimer, typically a heterodimer, that is composed of proteins belonging to the Fos/Jun subfamily (e.g. cFos, FRA1, FRA2, cJun, JUNB, JUND, GCN4, BATF, BATF2, BATF3).

In addition to the human bZIP transcription factors, certain viral proteins that bind DNA also belong to the bZIP family. This includes the bZIP transactivator of Epstein-Barr virus, BZLF1. BZLF1 can bind to either the TRE binding site (TGACTCA or TGAGTCA) or the CCAAT binding site (ATTGCGCAAT). The components of the DNA-binding protein may be, or may comprise, the DNA-binding fragment of BZLF1 or a DNA-binding fragment thereof, and the at least one binding site may be a TRE binding site or CCAAT binding site.

Components of DNA-binding proteins can also be derived from transcription factors that are not part of the bHLH, bZip or bHLH-Zip families. Examples of additional suitable eukaryotic transcription factors from which the components of DNA-binding proteins can be derived are set forth in the following table, along with the nucleotide sequences of their DNA binding sites and the names of these binding sites. The components of the DNA-binding protein may be derived from any of these eukaryotic (e.g. human) transcription factors and the reporter expression cassette comprise at least one of these binding sites set forth in the same row as the transcription factor in the table below.

| Eukaryotic transcription | | Nucleotide sequence(s) of |
|---|---|---|
| factor(s) | Name of binding site | binding site |
| CAAT-box binding factor* | CAAT box | GGCCAATCT |
| Serum response factor* | CArG box | CC(A/T)₆GG |
| Snail proteins (e.g. SNAH)* | E2 box | CAGGTG and CACCTG |
| Runx2* | HY box | TG(A/T)GGG |
| T box transcription factors* | T box | TCACACCT |
| RNA polymerase in eukaryotes* | TATA box | TATAAA |
| RFX proteins (e.g. RFX1)* | X box | GTTGGCATGGCAAC |
| Y box binding protein* | Y box | (A/G)CTAACC(A/G)(A/G)(C/T) |
| Ethylene-responsive element | ATA box | AAATAT |
| binding proteins | | |
| AtSR1 (Arabidopsis thaliana | CGCG box | (A/C/G)CGCG(C/G/T) |
| signal-responsive genes) | | |
| Dehydration-responsive element- | DREB box | TACCGACAT |
| binding (DREB)-like proteins | | |
| Fur protein | Fur box | GATAATGATAATCATTATC |
| EmBP1 | G box | GCCACGTGGC |
| EREBP-like proteins | GCC box | AGCCGCC |
| KAP-2 protein | H box | ACACCA |
| barley prolamin-box (P-box) | Prolamin box | TGTAAAG |
| binding factor | | |
| Aleurone proteins | Pyrimidine box | CCTTTT |
| U2 snRNP | TACTAAC box | ATTTACTAAC |

*Eukaryotic transcription factors that are also human transcription factors.

In some embodiments,
a) the at least one binding site is a TPA response element (TRE) having the nucleotide sequence TGACTCA (SEQ ID NO: 5) or TGAGTCA (SEQ ID NO: 6);
b) the at least one binding site is an Ebox response element having the nucleotide sequence CACGTG (SEQ ID NO: 7) or CACATG (SEQ ID NO: 8);
c) the at least one binding site is a CCAAT binding site having the nucleotide sequence ATTGCGCAAT (SEQ ID NO: 9);
d) the at least one binding site is a cAMP response element (CRE) having the nucleotide sequence TGACGTCA (SEQ ID NO: 10);
e) the at least one binding site is a Maf recognition element (MARE) having the nucleotide sequence TGCTGA^{G}/_{C}TCAGCA (SEQ ID NO: 32) or TGCTGA^{GC}/_{CG}TCAGCA (SEQ ID NO: 33); or
f) the at least one binding site is a PAP/CREB-2/PAR binding site having the nucleotide sequence TTACGTAA (SEQ ID NO: 34).

In particular embodiments,
a) the DNA-binding protein is a DNA-binding fragment of a member of the Fos/Jun subfamily of transcription factors (such as c-Jun), and the at least one binding site is a TPA response element (TRE) having the nucleotide sequence TGACTCA (SEQ ID NO: 5) or TGAGTCA (SEQ ID NO: 6);
b) the DNA-binding protein is a DNA-binding fragment thereof of c-Myc, and the at least one binding site is an Ebox response element having the nucleotide sequence CACGTG (SEQ ID NO: 7) or CACATG (SEQ ID NO: 8);
c) the DNA-binding protein is a DNA-binding fragment of a member of the C/EBP subfamily of transcription factors (such as C/EBP protein), and the at least one binding site is a CCAAT binding site having the nucleotide sequence ATTGCGCAAT (SEQ ID NO: 9);
d) the DNA-binding protein is a DNA-binding fragment of a member of the CREB subfamily of transcription factors (such as CRE), and the at least one binding site is a cAMP response element (CRE) having the nucleotide sequence TGACGTCA (SEQ ID NO: 10);
e) the DNA-binding protein is a DNA-binding fragment of a Maf transcription factor, and the at least one binding site is a Maf recognition element (MARE) having the nucleotide sequence TGCTGA^{G}/_{C}TCAGCA (SEQ ID NO: 32) or TGCTGA^{GC}/_{CG}TCAGCA (SEQ ID NO: 33);
f) the DNA-binding protein is a DNA-binding fragment of a member of the poly(ADP-ribose) (PAR) subfamily of transcription factors, and the at least one binding site is a PAP/CREB-2/PAR binding site having the nucleotide sequence TTACGTAA (SEQ ID NO: 34); or
g) the DNA-binding protein is a DNA-binding fragment of a member of the CREB-2 subfamily of transcription factors, and the at least one binding site is a PAP/CREB-2/PAR binding site having the nucleotide sequence TTACGTAA (SEQ ID NO: 34).

In particular embodiments, the DNA-binding protein is a DNA-binding fragment of a member of the Fos/Jun subfamily of transcription factors (such as c-Jun), and the at least one binding site is a TPA response element (TRE) having the nucleotide sequence TGACTCA (SEQ ID NO: 5) or TGAGTCA (SEQ ID NO: 6). In certain embodiments, the first and second component of the DNA-binding protein is basic c-Jun, i.e. a fragment containing the basic motif of c-Jun but lacking the leucine zipper dimerization domain. An exemplary amino acid sequence for basic c-Jun is set forth in SEQ ID NO: 47. Thus, in some embodiments, the first and second component of the DNA-binding protein comprise an amino acid sequence having at least 90%, 91%, 92%, 93%, 94%, 95%, 96%, 97%, 98%, 99%, or 100% identity to the amino acid sequence set forth in SEQ ID NO: 47. In some embodiments, the first and second component of the DNA-binding protein comprise the amino acid sequence of SEQ ID NO: 47, optionally with 1, 2, 3, 4, or 5 sequence alterations.

As described in the examples, a reporter expression cassette encoding murine DHFR as a reporter protein was generated, where the reporter expression cassette contained 15 TREs in its protein coding sequence. This exemplified protein coding sequence of this reporter expression cassette has the sequence set forth in SEQ ID NO: 4.

Thus, in some embodiments the reporter expression cassette comprises a protein coding sequence that is at least 90%, at least 95%, at least 98%, or 100% identical to the sequence set forth in SEQ ID NO: 4 and the DNA-binding protein is a DNA-binding fragment of a member of the Fos/Jun subfamily of transcription factors (such as c-Jun).

Hybrid Proteins

The hybrid proteins described herein comprise a component of the DNA-binding protein linked to a respective candidate binding partner. Provided are a “first hybrid protein”, which comprises the first component of the DNA-binding protein linked to the first candidate binding partner, and a “second hybrid protein”, which comprises the second component of the DNA-binding protein linked to the second candidate binding partner.

By “linked” is meant that the component of the DNA-binding protein is physically associated with the candidate binding partner, either covalently or non-covalently. Preferably the association is a covalent association. The component of the DNA-binding protein may be covalently linked to the N-terminus or C-terminus of its respective candidate binding partner.

The hybrid proteins may comprise the candidate binding partner and the respective component of the DNA binding protein within the same peptide chain. Such a hybrid protein may be regarded as a fusion protein, comprising both candidate binding partner and component of DNA binding protein. Thus either or both of the hybrid proteins may be fusion proteins.

The component of the DNA-binding protein may be separated from the candidate binding partner by a peptide linker, or the component of the DNA-binding protein may be fused directly to the candidate binding partner (i.e. without a peptide linker in between). Suitable peptide linkers include those represented by [G]n, [S]n, [A]n, [GS]n, [GGS]n, [GGGS]n, [GGGGS)n, [GGSG]n, [GSGG]n, [SGGG]n, [SSGG]n, [SSSG]n, [GG]n, [GGG]n, [SA]n, [TGGGGSGGGGS]n, and combinations thereof, wherein n is an integer between 1 and 30. For example, n may be 1, 2, 3, 4, 5, 6, 7, 8, 9, 10, or any number up to 30. The component of the DNA-binding protein and the candidate binding partner may be present in any relative orientation. For example, the component of the DNA-binding protein may be N-terminal or C-terminal of the candidate binding partner, with our without a linker in between. In some preferred embodiments, the component of the DNA-binding protein is N-terminal to the candidate binding partner.

As described herein, the first and second candidate binding partner may have an identical amino acid sequence or they may have different amino acid sequences. Similarly, the first and second components of the DNA-binding protein may have an identical amino acid sequence or they may have different amino acid sequences. Thus, in certain embodiments, the first and second hybrid proteins (e.g. first and second fusion proteins) have an identical amino acid sequence. Where the first and second proteins have an identical amino acid sequence, the DNA-binding complex is a homodimeric complex. Thus, the methods may be put into effect using a single expression cassette encoding just one such fusion protein, which is capable of homodimerizing once expressed.

Where the first and second hybrid proteins have different sequences, the methods of the invention will typically employ first and second expression cassettes, encoding the respective first and second hybrid proteins. Each expression cassette may comprise its own set of transcriptional and translational regulatory sequences to drive expression of the respective hybrid protein.

In some embodiments, where the first and second candidate binding partners are Aβ peptides, the first and second fusion proteins comprise an amino acid sequence that is at least 90%, 91%, 92%, 93%, 94%, 95%, 96%, 97%, 98%, 99% or 100% identical to the sequence set forth in SEQ ID NO: 51. In some embodiments, the first and second fusion proteins comprise the amino acid sequence of SEQ ID NO: 51. A fusion protein expression cassette encoding the first and second fusion proteins may comprise a nucleotide sequence that is at least 90%, 91%, 92%, 93%, 94%, 95%, 96%, 97%, 98%, 99% or 100% identical to the sequence set forth in SEQ ID NO: 50. In some embodiments, the fusion protein expression cassette comprises the nucleotide sequence of SEQ ID NO: 50.

In some embodiments, where the first and second candidate binding partners are αS polypeptides, the first and second fusion proteins comprise an amino acid sequence that is at least 90%, 91%, 92%, 93%, 94%, 95%, 96%, 97%, 98%, 99% or 100% identical to the sequence set forth in SEQ ID NO: 55. In some embodiments, the first and second fusion proteins comprise the amino acid sequence of SEQ ID NO: 55. A fusion protein expression cassette encoding the first and second fusion proteins may comprise a nucleotide sequence that is at least 90%, 91%, 92%, 93%, 94%, 95%, 96%, 97%, 98%, 99% or 100% identical to the sequence set forth in SEQ ID NO: 54. In some embodiments, the fusion protein expression cassette comprises the nucleotide sequence of SEQ ID NO: 54.

Also provided herein are fusion proteins comprising amyloid peptides, such as an amyloid-β (Aβ) peptide or an α-synuclein (αS) polypeptide, fused to a component of a DNA-binding protein. In one aspect, provided is a fusion protein comprising a component of a DNA-binding protein and an amyloid peptide capable of dimerization;

wherein said fusion protein forms a complex capable of binding DNA upon dimerization via the amyloid peptide component.

In some embodiments, the amyloid peptide components are amyloid-β (Aβ) peptides or α-synuclein (αS) polypeptides.

The fusion protein, components of the DNA-binding protein, and amyloid peptides may be as further defined herein.

Expression Cassettes

In this specification, the term “expression cassette” is intended to mean a DNA polynucleotide sequence that is capable of directing transcription of an expression product. The expression cassette and may be derived from a eukaryotic gene or a prokaryotic gene. A eukaryotic gene typically comprises, from 5′ to 3′, a promoter, a 5′ untranslated region (UTR), an open reading frame made up of exons and introns, a 3′ UTR and may further comprise one or more enhancers and/or silencers. Promoters are well known to be regions of DNA that are responsible for the initiation of transcription. Enhancers, are well known to be regions of DNA that can be bound by activator proteins to increase the likelihood that transcription will progress. Silencers are well known to be regions of DNA that can be bound by repressor proteins to decrease the likelihood that transcription will progress.

During transcription in eukaryotic cells, the eukaryotic gene is typically first transcribed into pre-mRNA in the nucleus of the cells, which contains the 5′ and 3′ UTRs and the exons and introns that make up the open reading frame. Following this, the pre-mRNA is processed into mRNA, which involves the addition of a 5′ cap to the beginning of the RNA, the addition of a poly-A tail to the end of the RNA and the removal of introns. The final mature mRNA is then able to travel out of the nucleus and be translated into a protein.

A prokaryotic gene has a similar structure, except it does not contain introns within the open reading frame. This means that the RNA transcript of a prokaryotic gene is ready to act as a mature mRNA and does not require the processing that features in eukaryotic cells. The transcription of an operon's mRNA is often controlled by a repressor that binds to a segment of DNA known as an operator. For example, the Lac operon encodes a repressor protein, which is under allosteric regulation. In the prokaryotic cell, the repressor protein is normally bound to the operator, which prevents transcription of the open reading frame. However, when the repressor is bound to the effector molecule lactose, or the structural analogue isopropyl β-D-1-thiogalactopyranoside (IPTG), the repressor will not bind to the operator, which allows transcription to occur. In this way, the initiation of transcription is dependent upon the availability of lactose or IPTG within the prokaryotic cell.

A “coding sequence” is intended to mean a portion of a gene's DNA sequence that encodes the expression product. Where the expression product is a protein, this sequence may be referred to as a “protein coding sequence”. The protein coding sequence typically begins at the 5′ end by a start codon and ends at the 3′ end with a stop codon. Furthermore, the protein coding sequence is typically the sequence of the gene exon(s) that in a gene is flanked by 5′ and 3′ UTRs. An example of a protein coding sequence is set forth in SEQ ID NO: 4.

Typically, the expression cassette comprises a promoter operably linked to a protein coding sequence. The term “operably linked” includes the situation where a selected coding sequence and promoter are covalently linked in such a way as to place the expression of the protein coding sequence under the influence or control of the promoter. Thus a promoter is operably linked to the protein coding sequence if the promoter is capable of effecting transcription of the protein coding sequence. Where appropriate, the resulting transcript may then be translated into a desired protein. In some embodiments, the expression cassette may further comprise further components of a eukaryotic or prokaryotic gene, such as one or more selected from the a list consisting of: an intron, an enhancer, a silencer, a 5′ UTR, a 3′ UTR, and a regulator.

Any suitable promoter known in the art may be used in the expression cassette providing it functions in the cell type being used. For example, where the cell is a bacterial cell, expression may be under control of the lac operon. In such cases, the cell may also contain a lac repressor protein, whereby expression can be controlled by the introduction of isopropyl β-D-1-thiogalactopyranoside (IPTG). The promoter may be endogenous to the cell in which the method is being carried out. Where multiple expression cassettes are used, each coding sequence may be independently operably linked to its own promoter. Alternatively, the coding sequence for one or more of the expression cassettes may be operably linked to the same promoter.

As already described here, the reporter expression product is encoded by a reporter expression cassette. The first and second fusion proteins may also be encoded by an expression cassette, termed herein a “fusion protein expression cassette”. Where the first and second fusion proteins have an identical amino acid sequence, a single fusion protein expression cassette may be used to encode the first and second fusion proteins. Where the first and second fusion proteins have different amino acid sequences, a first fusion protein expression cassette may encode the first fusion protein and a second fusion protein expression cassette may encode the second fusion protein. The test compound may also be a peptide or polypeptide that is expressed intracellularly from an expression cassette, termed herein a “test compound expression cassette”.

The expression cassettes described herein may be part of one or more expression vector(s). An “expression vector” as used herein is a DNA molecule used for expression of foreign genetic material in a cell. Any suitable vectors known in the art may be used. Suitable vectors include plasmids, binary vectors, viral vectors and artificial chromosomes (e.g. yeast artificial chromosomes). Alternatively, the expression cassettes described herein may be incorporated into the genome of the cell.

The methods described herein may comprise administering one or more expression cassettes described herein to the cell. For example, the method may comprise administering a reporter expression cassette, a fusion protein expression cassette, and/or a test compound expression cassette to the cell, optionally where the expression cassette(s) are part of one or more expression vector(s). Molecular biology techniques suitable for administering expression cassettes and producing proteins such as the fusion proteins and reporter protein described herein in cells are well known in the art, such as those set out in Sambrook et al., Molecular Cloning: A Laboratory Manual, New York: Cold Spring Harbor Press, 1989.

Reporter Expression Cassette and Binding Site(s)

As described above, the reporter expression cassette comprises at least one binding site such that binding of the DNA-binding protein to the binding site is capable of inhibiting expression of the reporter expression product. Preferably, the expression product comprises a plurality of such binding sites.

The at least one binding site may be located anywhere in the reporter expression cassette, providing binding of the DNA-binding protein to the at least one binding site is capable of inhibiting expression of the reporter expression product. For example, the binding site(s) may be located in a promoter, protein coding sequence, enhancer, silencer, 5′ UTR, 3′ UTR, regulator, exon, and/or intron. Binding of the DNA-binding protein to the binding site(s) may inhibit expression of the reporter expression product to less than 50%, less than 40%, less than 30%, less than 20%, less than 10%, or less than 5% of the expression of the reporter expression product when the cell comprises the reporter expression cassette without the DNA-binding protein.

Thus, in some embodiments the reporter expression cassette comprises at least 2, at least 3, at least 4, at least 5, at least 6, at least 7, at least 8, at least 9, at least 10, at least 11, at least 12, at least 13, at least 14, or at least 15 binding sites. Preferably, the reporter expression cassette comprises at least 2, more preferably at least 5, even more preferably at least 10, still more preferably at least 12, still further preferably at least 15 binding sites. In some embodiments, the reporter expression cassette comprises: between 1 and 20, between 1 and 18, between 1 and 15, between 1 and 10, between 1 and 5, between 2 and 20, between 2 and 18, between 2 and 15, between 2 and 10, between 2 and 5, between 5 and 18, between 5 and 10, between 10 and 18, or between 12 and 16 binding sites. In some embodiments, the reporter expression cassette comprises up to 5, up to 10, up to 15, up to 18, up to 20 binding sites. In an exemplified embodiment, the reporter expression cassette comprises 15 binding sites.

Preferably, some or all of the binding site(s) are located in the transcribed sequence of the reporter expression cassette, e.g. in the coding sequence of the reporter expression cassette. Even more preferably, the reporter expression cassette comprises a plurality of binding sites that are located in the transcribed sequence or coding sequence. Without wishing to be bound by theory, it is believed that a plurality of binding sites located in the transcribed region or coding sequence will increase the likelihood that binding of the DNA-binding protein to the binding sites will efficiently inhibit expression of the reporter expression product.

In embodiments where the reporter expression product is a reporter protein, it is preferable that the expression product is functional in order to determine whether the expression of the reporter protein is increased in the presence of the test compound of the screening method. In preferred embodiments, the presence of the binding site(s) in the reporter expression cassette does not substantially affect the function of the reporter protein. For example, the reporter protein may retain at least 50%, at least 70%, at least 90%, or at least 95% of the function of a parent reporter protein, wherein the parent reporter protein is encoded by a parent reporter expression cassette that corresponds to the reporter expression cassette but does not comprise the binding site(s).

In order to preserve the activity of the reporter protein, at least some of the binding site(s), preferably the majority of the binding site(s) may be introduced into the protein coding sequence of the reporter expression cassette as silent, semi-conservative and/or conservative mutations. The protein coding sequence is made up of a series of codons, each of which encodes a specific amino acid or stop signal when the protein coding sequence is transcribed and translated. Silent mutations are mutations in a codon of the protein coding sequence that do not affect the resulting amino acid residue of the codon. For example, the codon GCA encodes the amino acid Alanine (A). Mutating the GCA codon to GCG would be considered a silent mutation as the GCG codon still encodes the amino acid Alanine (A).

A conservative or semi-conservative mutation is a change to a given codon that leads to the replacement of one amino acid with a biochemically similar one, e.g. as set out according to the following table.

| | | |
|---|---|---|
| Hydrophobic | Alkyl | G A V L I M P |
| (non-polar) | Aromatic | F Y W |
| Hydrophilic | Neutral | S T C Q N |
| (polar) | Acidic | E D |
| | Basic | K H R |

For example, a change to a given codon that replaces a hydrophobic amino acid for another hydrophobic amino acid, or a hydrophilic amino acid for another hydrophilic amino acid, may be considered a semi-conservative mutation. For example, a change to a given codon that replaces a serine (S) to aspartic acid (D) may be considered a semi-conservative mutation. A change to a given codon that replaces an alkyl amino acid for another alkyl amino acid, or an aromatic amino acid for another aromatic amino acid, or a neutral amino acid for another neutral amino acid, or an acidic amino acid for another acidic amino acid, or a basic amino acid for another basic amino acid, may be considered a conservative mutation. For example, a change to a given codon that replaces a neutral, hydrophilic amino acid for another neutral, hydrophilic amino acid (e.g. threonine (T) to glutamine (Q)) may be considered a conservative mutation.

Thus, the reporter protein may have an amino acid sequence that is at least 80%, at least 85%, at least 90%, or at least 95% identical to a parent reporter protein, wherein the parent reporter protein is encoded by a parent reporter expression cassette that corresponds to the reporter expression cassette but does not comprise the binding site(s).

In some embodiments, the majority of the differences in the amino acid sequence of the reporter protein and the amino acid sequence of the parent reporter protein are conservative and/or semi-conservative substitutions. In these cases, it is expected that the reporter protein will have substantially the same function as the parent reporter protein.

The location of the binding site(s) in the reporter expression cassette may be selected so as to avoid affecting the function of the reporter protein. For example, the binding site(s) may be located at a position in the protein coding sequence that does not encode a residue that forms part, or is in close proximity to the catalytic centre (active site) of the reporter protein, or forms part, or is in close proximity to a residue involved in cofactor binding (e.g. NADH, NDDPH). Close proximity can mean that the residue is less than 15 Å, more preferably less than 10 Å, even more preferably less than 5 Å away from a residue that forms part of the catalytic centre and/or is involved in cofactor binding. Alternatively or additionally, close proximity can mean that the residue is less than 5 residues, more preferably less than 4 residues, even more preferably less than 3 residues, still more preferably less than 2 residues away from a residue that forms part of the catalytic centre and/or is involved in cofactor binding, when assessed in a linear sequence of amino acids.

Changes outside the catalytic centre of the reporter protein are expected to minimise functional alterations. Alternatively or additionally, the binding site(s) may be located at a position in the protein coding sequence that encodes a solvent exposed residue in the reporter protein. Changes made at solvent exposed regions of the reporter protein are expected to minimise the structural perturbations and therefore minimise perturbation to the overall function.

Methods of identifying the solvent exposed regions of the reporter protein are known. For example, it is possible to take the coordinate files for the reporter protein, e.g. a protein databank (PDB) file and use a program that calculates the accessible surface area (ASA) which informs the user how exposed/buried residues are within a structure. An exemplary ASA program can be found at http://cib.cf.ocha.ac.jp/bitool/ASA/. An exemplary cut-off value of 20 Å² can be used, such that residues that are lower than this are considered to be buried and greater than this are considered exposed. In this way, the locations of solvent exposed residues can be identified and codons modified accordingly.

In some embodiments, the reporter expression cassette encodes a reporter protein that is a fusion protein, where the fusion protein comprises two or more of the cell survival proteins, cell reproduction proteins, fluorescence proteins, bioluminescence proteins, enzymes that act on a substrate to produce a colorimetric signal, protein kinases, proteases, transcription factors, and regulatory proteins that are described herein. For example, the reporter expression cassette may encode a fusion protein comprising a cell survival protein as described herein and a fluorescence protein as described herein. In such an example, the binding site(s) may be located in the part of the reporter expression cassette (e.g. the coding sequence) that encodes the cell survival protein. This exemplary reporter expression cassette would therefore provide a two readouts of efficacy, namely cell survival and fluorescence. In a particular example, the fusion protein may comprise a DHFR as a cell survival protein and mNeonGreen as a fluorescence protein.

Cells

The method for screening of the invention functions in isolated live cells, i.e. the methods are performed in cellulo unless the context clearly dictates otherwise. The term “in cellulo” is intended to encompass experiments that take place involving cells and may be on cultured cells or may be on cells or tissues that have been taken from an organism. The methods of the invention are not practiced on the human or animal body.

As described above, many oligomers and confirmations of oligomers of peptides involved in protein aggregation are toxic. Thus, by carryout out the screening method in cellulo, this means that in addition to oligomer-driven transcriptional repression, the population of toxic oligomers will also result in toxicity and reduced cell growth rates, therefore improving the robustness of the assay. Where prokaryote cells are used, size of the colony produced may give a further indication of inhibitor activity and allow for the identification and removal of false negatives during subsequent selection.

Any cell suitable for the expression of expression products may be used for the screening method described herein. The cell may be a prokaryote or eukaryote. Typically the cells are isolated cells.

The cell used in the screening method may be a bacterial cell. In some embodiments, the bacterial cell is an Escherichia coli cell, for example BL21 (DE3), XL-1, RV308, or DH5alpha cells. Screening methods where the cell is a bacterial cell may involve culturing the bacterial cell in suitable media. Such techniques are well known to those of skill in the art.

Alternatively, the cell is a eukaryotic cell such as a yeast cell, a plant cell, insect cell or a mammalian cell. In some embodiments, the cell is a mammalian cell, for example a Chinese Hamster Ovary (CHO), or a human cell. Mammalian cells, especially human cells, may be somatic cells. Screening methods where the cell is a eukaryotic cell may involve culture or fermentation of the eukaryotic cell. The culture or fermentation may be performed in a bioreactor provided with an appropriate supply of nutrients, air/oxygen and/or growth factors. Culture, fermentation and separation techniques are well known to those of skill in the art.

Where the cell is a eukaryotic cell, and particularly a human cell, it may be a somatic cell. Typically the cell is not totipotent or pluripotent, e.g. it is not a single cell embryo or an embryonic stem cell.

As described above, a method for screening for an inhibitor of association between first and second candidate binding partners finds particular use in mammalian diseases associated with protein aggregation, such as neurodegenerative diseases including Alzheimer's Disease and Parkinson's Disease. Thus in some embodiments, the cell used is a mammalian neural cell, such as a human neuronal cell line.

Methods where the cell is a human cell have the additional advantage in that as well as screening for an inhibitor of association between first and second candidate binding partners, the method will simultaneously profile the test compound for further desirable properties that are conducive to drug development. For example, it can be used to determine if the test compound is toxic, if it is effectively able to inhibit dimerization of the components, and whether the test compound is stable in human cells. This compares favourably to known methods for identifying inhibitors of PPI that function as therapeutic compounds in human cells, where a first step would be to identify a PPI inhibitor, the second step to confirm that the PPI inhibitor ablates protein function and then the third step to check that it functions in human cells. The present invention therefore advantageously allows all these individual steps to be combined into an intracellular screening step in human cells.

For example, as described in the reporter protein section above, where the cell is a mammalian cell the reporter protein may be mammalian DHFR, e.g. murine DHFR that has been modified such that it is rendered resistant to the anti-folate drug methotrexate (MTX), for example as described by Remy et al. (2007). In this way, cell survival can be used as a readout to indicate whether the test compound is capable of inhibiting association between first and second candidate binding partners in question in the human cell.

Test Compound

The test compounds for use with the screening method of the invention are not particularly limited. In some embodiments, the test compound is peptidic. “Peptidic” as used herein includes compounds that are composed of or comprise a linear chain of amino acids linked by peptide bonds and include peptides and polypeptides. In this specification the term “peptide” is intended to mean molecules that consist of between 2 and 50 amino acids and the term “polypeptide” is intended to mean molecules that are made up of more than 50 amino acids. In other embodiments, the test compound is a small molecule, synthetic or naturally occurring. A small molecule is a compound (typically an organic compound) that has a molecular weight of 500 daltons or less.

In some embodiments, the test compound is a peptide mimetic. The terms “peptide mimetic”, “peptidomimetic” and “peptide analogue” are used interchangeably and refer to a chemical compound that is not entirely composed of amino acids but has substantially the same characteristics as a peptidic compound that is entirely composed of amino acids. A peptide mimetic may be peptidic, in that it is a chimeric molecule that it is made up of both natural peptide amino acids and non-natural analogues of amino acids. Alternatively, a peptide mimetic may not be peptidic, in that it is entirely composed of synthetic, non-natural analogues of amino acids. Peptide mimetics may be classified as set out in Pelay-Gimeno et al. (2015). Briefly ‘class A’ mimetics correspond to peptidic compounds that are mainly formed by amino acids with minor side chain or backbone alterations; ‘class B’ mimetics correspond to peptidic compounds with various backbone and side chain alterations; ‘class C’ mimetics correspond to small molecule-like scaffolds that project substituents in analogy to peptide side chains; and ‘class D’ mimetics correspond to molecules that mimic the mode of action of a peptide without a direct link to its side chains.

In some embodiments, the test compound is a peptidic test compound that is expressed intracellularly from a nucleotide sequence. For example, the nucleotide sequence may be an expression cassette (also termed a “test compound expression cassette”, which may be contained in a vector present in the cell, or may be incorporated into the genome of the cell as described above.

The screening method of the invention is expected to have use with genetically encoded peptidic libraries. Genetically encoded peptidic libraries are known and have been used in screening methods for identifying inhibitors of proteins. See, for example, Mern et al. (2010). Briefly, such libraries are formed from libraries of test compound expression cassettes, each of which encodes and is capable of directing expression of a different peptidic test compound. By transforming the library into cells containing the first and second fusion proteins, and reporter expression cassette, it is possible to indicate whether a given library member is capable of inhibiting association between first and second candidate binding partners. Such genetically encoded peptidic libraries can be used with the method of the present invention to rapidly screen multiple different test compounds at the same time.

Thus in some embodiments, the cell used in the method was obtained from a pool of cells that were transformed with a genetically encoded library of peptidic test compounds, such that the cell expresses the peptidic test compound intracellularly.

The present inventors have also recognised that the screening method can be used with test compounds that are added extracellularly. For example, cells containing the first and second fusion proteins, and reporter expression cassette can be cultured and plated onto microtiter plates (e.g. 1536 well plates) and test compound libraries screened by direct addition to each well. Addition of the test compound libraries to the wells can occur before or after addition of the cells. This method can be used to rapidly screen multiple different test compounds and has the additional advantage of allowing the user to move away from standard peptide libraries, for example allowing the user to profile for helix constrained peptides, peptidomimetics, non-natural amino acids, or even small molecule libraries. Test compounds that are added extracellularly must be able to cross the cell membrane (and cell wall, if present) in order to enter the cell and be screened to indicate if they are capable of preventing association between first and second candidate binding partners using the methods of the invention. This means that the extracellular test compound addition method allows the user to profile for cell penetrance concomitantly with inhibition of dimerization as an increase in expression of the reporter expression product will indicate that the test compound is capable of entering the cell and capable of inhibiting DNA-binding activity of the DNA-binding protein. Compounds used in a therapeutic setting in humans will need to enter the cells in order to have a therapeutic effect. Thus, without wishing to be bound by theory, it is expected that those extracellularly-added test compounds that result in an increase in expression of reporter expression product using the methods described herein represent good candidates for taking forward as potential therapeutic agents. Furthermore, because cell penetrance and dimerization inhibition is determined concomitantly, this compares favourably to methods that require separate assays to test for cell penetrance and for in cellulo dimerization inhibition.

Thus, in some embodiments, the method comprises administering the test compound extracellularly in order to obtain a cell that comprises the test compound. For example, the test compound may be added to culture media that the cell is being cultured in. In embodiments where the test compound is administered extracellularly, an increase in expression of the reporter expression product indicates that the test compound is capable of entering the cell as well as being capable of inhibiting association between first and second candidate binding partners.

In some embodiments, the test compound is one that has previously been identified as being able to interact with first and/or second candidate binding partner, or is suspected of being able to inhibit association between the first and second candidate binding partners. For example, the test compound may be suspected to be an inhibitor based on a PCA assay and the method described herein can then be used to provide further indication that the inhibitor is capable of inhibit association between the first and second candidate binding partners.

In some embodiments, the method may further comprise carrying out an in vitro assay to confirm binding of the test compound to the first and/or second candidate binding partner. This can be used, for example, to distinguish those test compounds that are inhibiting dimerization by binding to the first and/or second candidate binding partner from those test compounds that are inhibiting dimerization by binding to the first and/or second components of the DNA-binding protein. This can be carried out using any method known in the art for detecting binding between two or more proteins. For example, the in vitro assay may comprise carrying out one or more of surface plasmon resonance (SPR), isothermal calorimetry and X-ray crystallography.

The method may further comprise, carrying out biophysical, structural and/or cell-based approaches to further analyse function of the test compound. For example, circular dichroism spectroscopy experiments can be carried out to detect changes in global secondary structure following addition of the test compound. Single molecule fluorescence and atomic force microscopy (AMF) can be carried out, e.g. to confirm prevention of amyloid formation by the test compound. Further analysis may be carried out in mammalian cells, e.g. neuronal mammalian cells where aggregation of the first and second candidate binding partners in a human patient is associated with a neurodegenerative disease. For example, continuous growth ThT experiments can be carried out in primary neuron cells to demonstrate inhibition of amyloid formation in a dose-dependent manner, neuronal cell assays carried out to demonstrate reduced cytotoxicity of the candidate binding partners, and/or intracellular delivery of peptides to test colocalization and downstream effects of the test compounds on cytotoxicity and proteostasis in wild-type or mutant neurons.

The residues present on the surface of a protein that are responsible for PPIs are associated with protein secondary structure motifs, such as alpha-helix, beta-sheets and beta-turns. In some embodiments, the test compound comprises an alpha-helix, such as a helix-constrained peptide. In some embodiments, the test compound may comprise a beta-strand, which may form a beta-sheet.

The term “helix-constrained peptide” is intended to mean a peptide having at least one chemical modification that results in an intramolecular cross-link between two amino acids in order to produce a stabilised alpha-helix. Generally, the cross-link extends across the length of one or two helical turns (i.e. about 3-3.6 or about 7 amino acids). Accordingly, amino acids positioned at i and one of: i+3, i+4, and i+7 are ideal candidates for cross-linking. Thus, for example, where a peptide has the sequence . . . X1, X2, X3, X4, X5, X6, X7, X8, X9, . . . and the amino acid X is independently selected for each position, cross-links between X1 and X4, or between X1 and X5, or between X1 and X8 are useful as are cross-links between X2 and X5, or between X2 and X6, or between X2 and X9, etc. The use of multiple cross-links (e.g., 2, 3, 4 or more) is also contemplated.

Chemical modification includes a chemical modification to incorporate a molecular tether, such as a hydrocarbon staple, and a chemical modification to promote the formation of a disulphide bridge. The cross-link can be an ionic, covalent or hydrogen bond that links the two residues together, preferably the cross-link is a covalent bond.

The presence of a stabilised alpha-helix can be determined using methods such as circular dichroism spectroscopy for an alpha-helix, for example as described in Jo et al. (2012). Circular dichroism be used to measure a helicity increase, i.e. linear to cyclic. In situations where the cross-linking occurs through the formation of a disulphide bridge between two thiol groups, such as between two cysteine residues, the presence of a stabilised alpha-helix can also be determined using an assay that determining if thiols in the sample are free or conjugated. For example, free thiols can be assayed via reaction with Ellman's reagent (5,5′-dithiobis(2-nitrobenzoic acid; DNTB) (Sigma)) and monitoring absorbance at 412 nm.

Methods of inducing cross-links between amino acids are well known and include methods that induce cross-links between the peptide backbone, e.g. between the carbonyl group and amino group as in natural alpha-helices, as well as between side-chains of the peptides. Methods include disulphide bond formation (e.g. as described in Leduc et al. (2003)), hydrogen bond surrogates (e.g. as described in Wang et al. (2005)), ring-closing metathesis (e.g. as described in Walensky et al. (2004)), cysteine alkylation using α-haloacetamide derivatives (e.g. as described in Woolley (2005)) or biaryl halides (e.g. as described in Muppidi et al. (2011)), lactam ring formation (e.g. as described in Fujimoto et al. (2008)), hydrazine linkage (e.g. as described in Cabezas & Satterthwait (1999)), oxime linkage (e.g. as described in Haney et al. (2011)), metal chelation (e.g. as described in Ruan et al. (1990)), and “click” chemistry (e.g. as described in Holland-Nell & Meldal (2011)).

In some embodiments, the cross-link is introduced between the amino acids in the peptidic test compound to produce a helix-constrained peptide prior to administering the test compound to the cell, e.g. administering the test compound extracellularly.

The present inventors have also made the surprising discovery that it is possible to introduce the intra-molecular cross-link into the test compound intracellularly. Thus, a method where the peptidic test compound are cross-linked during the intracellular selection step could be used to directly screen for helix-constrained peptides within the cell. Since the helix-constrained peptide is present within the cell, the cells can immediately be used for subsequent screening for whether the test compound is capable of inhibiting association between the first and second candidate binding partner using the screening method described herein. Furthermore, this method is applicable for polypeptides that contain the helix-constrained peptide, allowing the helix-constrained peptide to be screened to determine if it can disrupt FP's in the context of the polypeptide.

Thus, in some embodiments of the screening method described herein where the test compound comprises a peptide, the method further comprises administering a cross-linking agent into the cell, wherein the cross-linking agent chemically modifies the peptide to introduce a cross-link between two amino acid residues to produce a stabilised alpha-helix, thereby producing the test compound comprising the helix-constrained peptide. The test compound may be expressed intracellularly from a test compound expression cassette.

In some embodiments, the cross-link is formed between amino acids at positions i and i+3, i and i+4, or i and i+7 in the amino acid sequence of the peptide. In some embodiments, the cross-link is between cysteine (C) residues located at these positions. In other embodiments, the cross-link is between lysine (K) and aspartic acid (D) residues at these positions. Preferably, the cross-link is formed between amino acids at positions i and i+4.

In some embodiments, the method comprises determining expression of the reporter expression product both before and after the addition of the cross-linking agent. In this way, it can be determined whether the peptide or polypeptide is capable of inhibiting association between the first and second candidate binding partner both before and after cross-linking, therefore providing an indication of the functional effect that constraining the alpha-helix in the peptide is having.

In preferred embodiments, the peptide comprises a cysteine (C) at positions i and i+4 in its amino acid sequence. As described in Jo et al. (2012), the introduction of cysteine residues at i and i+4 positions is useful because this spacing brings two thioether residues into proximity when in the alpha-helix. Suitable cross-linking agents for stabilising the alpha-helix within the peptide containing a cysteine (C) at position i and i+4 are described in Jo et al. (2012). For example, the cross-linking agent could be a cross-linker selected from the group consisting of an alkyl bromide, an alkyl iodide, a benzyl bromide, an allyl bromide, a maleimide, and an electrophilic difluorobenzene. In preferred embodiments, the cross-linking agent is an m-xylene based, o-xylene based, or p-xylene based benzyl bromide, more preferably a m-xylene based benzyl bromide. In particularly preferred embodiments, the cross-linking agent is 1,3-dibromomethylbenzene (DBMB) having the following chemical formula:

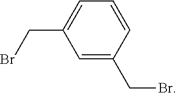

In some embodiments, the peptide comprises a lysine (K) and aspartic acid (D) at i and i+4 positions in its amino acid sequence. That is, position i is a lysine (K) and position i+4 is an aspartic acid (D), or position i is an aspartic acid (D) and position i+4 is a lysine (K). Methods of carrying out K-D lactamisation are described, for example, in de Araujo et al. (2014).

The method may comprise adding the cross-linking agent at a pH of between 7.5 and 8.5, preferably a pH of 8.0. This can be achieved using various buffers, as is well understood in the art. The method may additionally comprise treating the cells with tris(2-carboxyethyl) phosphine (TCEP), which may help drive specific bi-alkylation. In particular exemplary methods, the DBMB cross-linking agent may be added to the test compound comprising a helix-constrained peptide with TCEP and ammonium bicarbonate, and reacted at pH 8.0 and room temperature for 4 to 5 hours in the dark.

Cell-Free Method

Although the methods of the invention have been described primarily in the context of assays in cellulo, it will be clear that they can equally be performed in cell-free expression systems, and the disclosure relating to methods in cellulo should be construed accordingly except where the context requires otherwise.

Thus, the present invention provides a cell-free method for screening for an inhibitor of association between first and second candidate binding partners, the method comprising:

providing a cell-free expression system comprising

a test compound;

a first hybrid protein comprising a first component of a DNA-binding protein linked to the first candidate binding partner;

a second hybrid protein comprising a second component of the DNA-binding protein linked to the second candidate binding partner; and

a reporter expression cassette that encodes a reporter expression product,

wherein the first and second hybrid proteins form a DNA-binding complex upon association of the first

and second candidate binding partners, and wherein the reporter expression cassette comprises at least

one binding site for the DNA-binding complex such that binding of the DNA-binding complex to the

binding site inhibits expression of the reporter expression product; and

determining expression of the reporter expression product.

An increase in expression of the reporter expression product in the presence of the test compound typically indicates that the test compound is capable of inhibiting association between first and second candidate binding partners.

Such methods are carried out using in vitro expression systems comprising the components required for expression of the reporter. Although described as “cell free”, cells may be present. However, expression of the reporter does not take place within cells. Such expression systems contain the molecular components required to synthesise proteins from DNA in vitro, including RNA polymerase, ribosomes, tRNAs, amino acids, initiation, elongation and termination factors, etc. and only require the addition of template DNA. Commercially available in vitro transcription-translation kits can be used. An example of a commercially available in vitro transcription-translation kits is the PURExpress® in vitro Protein Synthesis Kit available from New England Biolabs (Catalogue number E6800).

In such cell-free methods, the reporter protein can be any protein that provides an observable phenotype, for example a fluorescent reporter protein or a protein that provides a colorimetric signal. Further details about suitable reporter proteins are described above.

Alternatively, the reporter protein could be DHFR and NADPH could be monitored in order to determine protein expression. DHFR is an enzyme that reduces dihydrofolic acid to tetrahydrofolic acid, using NADPH as electron donor, meaning that as tetrahydrofolic acid is produced NADPH is oxidised to NADP+. The oxidation of NADPH to NADP+ is accompanied by a decrease in absorbance at 340 nM (A340), which can be monitored by spectrophotometry. Thus, when the reporter protein is DHFR, an increase in protein expression can revealed by a decrease in absorbance at 340 nM

Antagonists, Cells, Kits and Libraries

In some embodiments, the methods for screening described herein further comprise isolating the test compound that has been indicated as being capable of inhibiting association between first and second candidate binding partners. Isolated test compounds identified by the methods of the present invention therefore form further aspects of the present invention.

As noted above, inhibitors that are capable of inhibiting association between first and second candidate binding partners may be useful in a therapeutic setting. Thus, the inhibitors may have utility in the treatment per se as pharmaceuticals, or may be valuable lead compounds for modification and improvement. In either case such pharmaceutical compounds, including modified or improved compounds, form further aspects of the present invention.

Thus the aspects of the invention described above may further comprise the step of formulating the inhibitor identified by the screen with a pharmaceutically acceptable excipient. The pharmaceutical compositions encompassed by the invention may be formulated and administered by any number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual, or rectal means.

In another aspect, the present invention provides a kit comprising:

a reporter expression cassette that encodes a reporter expression product; and

one or more fusion protein expression cassettes encoding a first and second fusion protein,

wherein the first fusion protein comprises a first component of a DNA-binding protein and a first candidate binding partner,

wherein the second fusion protein comprises a second component of a DNA-binding protein and a second candidate binding partner,

wherein the first and second fusion proteins form a DNA-binding complex upon association of the first and second candidate binding partners; and

wherein the reporter expression cassette comprises at least one binding site for the DNA-binding complex such that binding of the DNA-binding complex to the binding site inhibits expression of the expression product.

In some embodiments, the reporter expression cassette comprises a coding sequence having a nucleotide sequence that is at least 90%, at least 95%, at least 98%, or 100% identical to the sequence set forth in SEQ ID NO: 4.

In some embodiments, the kits defined above further comprise a test compound, which may be a peptide, polypeptide or test compound as described above. Where the test compound is a peptide or polypeptide, the test compound may be expressed from a test compound expression cassette. That is, in some embodiments, the kit further comprises a test compound expression cassette that encodes a test compound peptide or polypeptide.

One or more of the reporter expression cassette, fusion protein expression cassette(s), and test compound expression cassette (where present) in the kit may be part of one or more expression vector(s). For example, the kit may comprise a reporter expression vector that comprises the reporter expression cassette, one or more fusion protein expression vector(s) that comprise the one or more fusion protein expression cassette(s), and optionally a test compound expression vector that comprises the test compound expression cassette. Where the first and second fusion proteins have an identical amino acid sequence and are both encoded from the same fusion protein expression cassette, the kit may comprise a reporter expression vector that comprises the reporter expression cassette, a fusion protein expression vector that comprises the fusion protein expression cassette, and optionally a test compound expression vector that comprises the test compound expression cassette. The kit may comprise a single expression vector that comprises the reporter expression cassette, the first and/or second fusion protein expression cassette, and optionally the test compound expression cassette.

The first and second fusion proteins, reporter expression cassette, reporter expression product and test compound may be as further described above.

In another aspect, the present invention provides a cell comprising:
i) a reporter expression cassette that encodes a reporter expression product; and
ii) one or more fusion protein expression cassettes encoding a first and second fusion protein,

wherein the first fusion protein comprises a first component of a DNA-binding protein and a first candidate binding partner,

wherein the second fusion protein comprises a second component of a DNA-binding protein and a second candidate binding partner,

wherein the first and second fusion proteins form a DNA-binding complex upon association of the first and second candidate binding partners; and
wherein the reporter expression cassette comprises at least one binding site for the DNA-binding complex such that binding of the DNA-binding complex to the binding site inhibits expression of the expression product.

In some embodiments, the reporter expression cassette comprises a coding sequence having a nucleotide sequence that is at least 90%, at least 95%, at least 98%, or 100% identical to the sequence set forth in SEQ ID NO: 4.

In some embodiments, the cell further comprises a test compound expression cassette that encodes a test compound, wherein the test compound is a peptide or polypeptide. The invention also provides a genetically encoded library comprising a plurality of these cells, wherein each cell comprises a different test compound expression cassette.

One or more of the reporter expression cassette, fusion protein expression cassette(s), and test compound expression cassette (where present) in the cell may be part of one or more expression vector(s). For example, the cell may comprise a reporter expression vector that comprises the reporter expression cassette, one or more fusion protein expression vector(s) that comprise the fusion protein expression cassette, and optionally a test compound expression vector that comprises the test compound expression cassette. Where the first and second fusion proteins have an identical amino acid sequence and are both encoded from the same fusion protein expression cassette, the cell may comprise a reporter expression vector that comprises the reporter expression cassette, a fusion protein expression vector that comprises the fusion protein expression cassette, and optionally a test compound expression vector that comprises the test compound expression cassette. The cell may comprise a single expression vector that comprises the reporter expression cassette(s), the fusion protein expression cassette, and optionally the test compound expression cassette. One or more of the reporter expression cassette, fusion protein cassette(s) and test compound expression cassette may be incorporated into the genome of the cell.

The first and second fusion proteins, reporter expression cassette, reporter expression product and test compound may be as further described above.

In another aspect, the present invention provides a kit comprising a cell as defined above.

Sequence Identity and Alterations

Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps. Generally, default parameters are used, with a gap creation penalty equaling 12 and a gap extension penalty equaling 4. Use of GAP may be preferred but other algorithms may be used, e.g. BLAST (which uses the method of Altschul et al. (1990)), FASTA (which uses the method of Pearson and Lipman (1988)), or the Smith-Waterman algorithm (Smith and Waterman (1981)), or the TBLASTN program, of Altschul et al. (1990) supra, generally employing default parameters. In particular, the psi-Blast algorithm may be used.

Where the disclosure makes reference to a particular amino acid sequence having at least 90% sequence identity to a reference amino acid sequence, this includes the amino acid sequence having 90%, 91%, 92%, 93%, 94%, 95%, 96%, 97%, 98%, 99% and 100% sequence identity to the reference amino acid sequence.

The term “sequence alterations” as used herein is intended to encompass the substitution, deletion and/or insertion of an amino acid residue. Thus, a protein containing one or more amino acid sequence alterations compared to a reference sequence contains one or more substitutions, one or more deletions and/or one or more insertions of an amino acid residues as compared to the reference sequence. In some embodiments in which one or more amino acids are substituted with another amino acid, the substitutions may be conservative or semi-conservative substitutions, as further described above.

In some embodiments, substitution(s) may be functionally conservative. That is, in some embodiments the substitution may not affect (or may not substantially affect) one or more functional properties (e.g. binding affinity) of the protein comprising the substitution as compared to the equivalent unsubstituted protein.

The invention includes the combination of the aspects and preferred features described except where such a combination is clearly impermissible or expressly avoided.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### SUMMARY OF THE FIGURES

Embodiments and experiments illustrating the principles of the invention will now be discussed with reference to the accompanying figures in which:

FIG. 1. General principles of the Transcription-Block Survival (TBS) Assay.

Fifteen TREs have been introduced into the mDHFR gene. A) The changes to the DNA sequence result in a fully active protein that expresses, folds, and confers survival in M9 minimal media using trimethoprim (TMP) to inhibit bacterial DHFR. B) Basic-cJun can form DNA-bound homodimers and its expression prevents mDHFR transcription and no colonies grow. M9 agar plates with trimethoprim in the absence of IPTG (left hand side plates) do not form colonies. Expressing mDHFR with IPTG (right hand side plates), generates colonies in A) but not B).

FIG. 2. Schematic of the amyloid-TBS assay for detecting inhibitors of αS dimerization

This intracellular assay utilises BL21 E. coli expressing basic-αS and a reporter plasmid, mDHFR. DHFR is an essential protein and under the specific inhibition of the endogenous bacterial form using the antibiotic Trimethoprim (Tmp), the transcription and subsequent expression of mDHFR is essential for cell survival. BL21 E. coli are co-transformed with 2 plasmids. Firstly, the mDHFR plasmid which contains silent mutations that preserve the native structure and function of the expressed mDHFR protein but provides the specific 2-O-tetradecanoylphorbol-13-acetate response element (TRE) DNA-binding motifs for the basic regions of c-Jun to bind. Secondly, Basic-αS, encoding recombinant αS with the DNA-binding basic regions of c-Jun attached to the N-terminus. On aggregation of basic-αS, the basic regions come together and bind the TRE binding sites in the mDHFR reporter plasmid. This prevents DNA polymerase progression and halts mDHFR transcription, causing cell death. As a cell death signal confers primary events of the αS misfolding cascade and aggregation, successful inhibitors of these events will allow mDHFR transcription and can be identified via cell survival and growth of colonies. Basic regions adapted from PDB 1 a02 using Swiss-PdbViewer (Version 4.1.0). CmR, chloramphenicol resistance; AmpR, ampicillin resistance.

FIG. 3. The amyloid-TBS assay can be used to identify compounds that bind and sequester αS peptides as monomers.

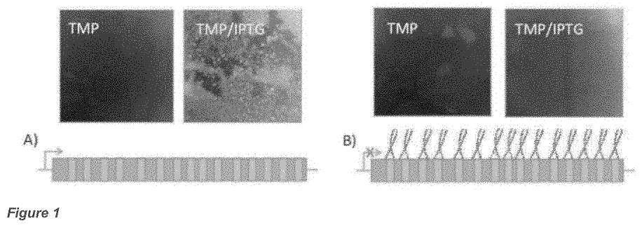

A) Fifteen TREs have been introduced into the mDHFR gene and this results in a fully active protein as described in FIG. 1A. B) Basic-αS can form DNA-bound homodimers and its expression prevents mDHFR transcription and no colonies grow. C) Peptides that bind to the aggregate form of αS but that fail to target the monomer result in paired DNA-binding domains which therefore do not dissociate the DNA-bound complex. This will not rescue transcription of the mDHFR gene and no colonies are observed. D) Inhibitor expression results in the basic-αS complex dissociating from TRE sites on the mDHFR gene leading to the restoration of mDHFR transcription-translation and colony formation. Consistent with this result, M9 agar plates with trimethoprim in the absence of IPTG (left hand side plates) do not form colonies. Expressing mDHFR with IPTG (right hand side plates), generates colonies in A) and D) but not B) or C).

FIG. 4. Schematic of the Aβ₁₋₄₂ dimerization assay

Schematic illustrating general principles of using the TBS assay to screen for inhibitors of the initial dimerization event of Aβ₁₋₄₂. In bacterial cells, endogenous DHFR can be inhibited using the antibiotic trimethoprim, making cells reliant on the modified, exogenous DHFR (top panel). In this setting, if AP-1 binds to the TRE sites in the DHFR, transcription is blocked and no functional DHFR enzyme is produced, resulting in cell death (middle panel). By attaching this basic cJun region to Aβ₁₋₄₂, a functional DNA binder will be created if two Aβ₁₋₄₂ peptides dimerize. In the absence of an inhibitor, basic-Aβ₁₋₄₂ dimerization would lead to blocking of the DHFR gene transcription, resulting in cell death (lower left hand panel). Only cells treated with a successful Aβ₁₋₄₂ dimerization inhibitor would produce DHFR and so survive, allowing selection for potential therapeutics for treating Alzheimer's Disease (lower right hand panel).

### EXAMPLES

### Example 1—Development of a Generalised Approach to Identify Inhibitors of Dimerization

Many rational design approaches, randomised screening approaches, and selection systems result in the successful identification of compounds capable of binding to given protein targets. However, what is much more difficult to ensure, is that binding to said target will result in ablating target protein function. There are many instances where formation of a protein-protein interaction (PPI) has not ensured loss of function. To address this major bottleneck in antagonist screening and design, we have taken inspiration from the transcription factor DNA-binding system and reversed their role in transcription.

Introducing DNA-Binding Sites into the DHFR Gene

It can be difficult to predict whether a compound that is derived to bind to given protein target will antagonise its function. To tackle this we have taken the gene corresponding to the essential enzyme, dihydrofolate reductase (DHFR), and introduced 15 TPA response elements (TREs) into the gene. This has been achieved using a combination of both silent and conserved mutations, such that the activity of the enzyme is preserved.

All changes have been made in solvent exposed regions of the molecule to minimise the structural perturbations, with several proposed changes removed via close inspection of the accessible surface area (ASA) within the pdb file (PDBid=2FZJ (Cody et al. (2006)). This was done by inputting the pdb file into the ASA calculator at http://cib.cf.ocha.ac.jp/bitool/ASA/. A cut-off value of 20 was used—residues that had an ASA value lower than this were considered to be buried and not modified; residues that had an ASA value greater than this are considered exposed.

No changes have been made in residues deemed important for catalysis or NADPH binding. Methods of identifying the solvent exposed regions of the reporter protein are known. For example, it is possible to take the coordinate files for the reporter protein, e.g. a protein databank (PDB) file and use a program that calculates the accessible surface area (ASA) which informs the user how exposed/buried residues are within a structure. An exemplary ASA program can be found at http://cib.cf.ocha.ac.jp/bitool/ASA/. An exemplary cut-off value of 20 can be used, such that residues that are lower than this are considered to be buried and greater than this are considered exposed. In this way, the locations of solvent exposed residues can be identified and codons modified accordingly.

Shown below is the sequence of the mDHFR gene (SEQ ID NO: 11) with DNA mutations bold and underlined and changes within the translated protein sequence (SEQ ID NO: 31) shown. Shown in bold italics are the NheI and HindIII sites used for subcloning the gene into the pES300d vector. Mutations were made by inspection of the desired consensus sequences (TGACTCA or TGAGTCA) and all three frames and the corresponding changes to the amino acid sequence upon making the necessary single base-pair changes. For example, either of the two desired sequences above can be put into any one of the three reading frames and the corresponding amino acid sequence and tolerated variations can be given:

| | | | | | | | |
|---|---|---|---|---|---|---|---|
| i) | Frame 1: | TGA | CTC | Axx | 1 = stop | 2 = LV | 3 = I/M/T/N/K/S/R |
| ii) | Frame 2: | xTG | ACT | CAx | 1 = LMV | 2 = TS | 3 = HQ |
| iii) | Frame 3: | xxT | GAC | TCA | 1 = FSYCLPHRITNVADG | 2 = DE | 3 = S |

This gives rise to a number of codons to be identified for silent mutation and consequently a number of options for conserved or semi-conserved mutations that would permit the introduction of TREs into the mDHFR gene:
i) No options
ii) LSH, LSQ, LTH, LTQ, MSH, MSQ, MTH, MTQ, VSH, VSQ, VTH, VTQ
iii) ADS, AES, CDS, CES, DDS, DES, FDS, FES, GDS, GES, HDS, HES, IDS, IES, LDS, LES, NDS, NES, PDS, PES, RDS, RES, SDS, SES, TDS, TES, VDS, VES, YDS, YES

From this we were able to implement the following changes into the mDHFR gene to give minimum perturbation to the overall sequence. Where possible mutations were silent or conservative. All mutations were also placed at solvent exposed sites and away from the catalytic centre (E116) and away from residues required for NADPH/substrate binding (A10/R71). This resulted in the introduction of 15 TREs into the mDHFR gene:

| | | | |
|---|---|---|---|
| 1. | VSQ | (silent) | = GTG AGT CAG |
| 2. | NEF→NES | (F32S) | = AAT GAG TCA |
| 3. | MTT→MTQ | (T40Q) | = ATG ACT CAG |
| 4. | TSS→TDS | (S42D) | = ACT GAC TCA |
| 5. | VEG→VES | (G46S) | = GTT GAG TCA |
| 6. | PEK→PES | (K64S) | = CCT GAG TCA |
| 7. | LSR→LSQ | (R78Q) | = CTG AGT CAA |
| 8. | IEQ→IES | (Q103S) | = ATT GAG TCA |
| 9. | VDM→VDS | (M112S) | = GTT GAC TCA |
| 10. | MNQ→MTQ | (N127T) | = ATG ACT CAA |
| 11. | VTR→VTQ | (R138Q) | = GTG ACT CAG |
| 12. | FES | (silent) | = TTT GAG TCA |
| 13. | IDL→IDS | (L154S) | = ATT GAC TCA |
| 14. | PEY→PES | (Y163S) | = CCT GAG TCA |
| 15. | LSE→LSQ | (E169Q) | = CTG AGT CAG |

This design process gave rise to the following sequence:

A S V R P L N C I V A V S Q N M G [symbol]
GTT CGA CCA TTG AAC TGC ATC GTC GCC [symbol] AAT ATG GGG
I G K N G D L P W P P L R N E S K
ATT GGC AAG AAC GGA GAC CTA CCC TGG CCT CCG CTC AGG [symbol] AAG
Y F Q R M T Q T D S V E S K Q N L
TAC TTC CAA AGA [symbol] [symbol] [symbol] AAA CAG AAT CTG
V I M G R K T W F S I P E S N R P
GTG ATT ATG GGT AGG AAA ACC TGG TTC TCC ATT [symbol] AAT CGA CCT
L K D R I N I V L S Q E L K E P P
TTA AAG GAC AGA ATT AAT ATA GTT [symbol] GAA CTC AAA GAA CCA CCA
R G A H F L A K S L D D A L R L I
CGA GGA GCT CAT TTT CTT GCC AAA AGT TTG GAT GAT GCC TTA AGA CTT [symbol]
E S P E L A S K V D S V W I V G G [symbol]
CCG GAA TTG GCG AGC AAA [symbol] GTT TGG ATC GTC GGA GGC
S S V Y Q E A M T Q P G H L R L F
AGT TCT GTT TAC CAG GAA GCC [symbol] CCA GGC CAC CTT AGA CTC TTT
V T Q I M Q E F E S D T F F P E I [symbol]
ATC ATG CAG GAA [symbol] GAC ACG TTT TTC CCA GAA [symbol]
D S G K Y K L L P E S P G V L S Q [symbol]
GGG AAA TAT AAA CTT CTC [symbol] CCA GGC GTC [symbol]
V Q E E K G I K Y K F E V Y E K K
GTC CAG GAG GAA AAA GGC ATC AAG TAT AAG TTT GAA GTC TAC GAG AAG AAA
D * A *
GAC T [symbol] AA

We have introduced 15 TREs via silent and conserved mutations into solvent exposed positions within the gene coding for the essential enzyme dihydofolate reductase (DHFR). We demonstrate that these changes result in a functional enzyme. Under selective conditions introduction of AP-1 prevents DHFR expression by binding to TRE sites within the gene, blocking transcription, and preventing colony formation under selective conditions (FIG. 1A). In contrast, attenuated versions of AP-1 that lack a basic DNA-binding region fail to prevent colony formation.

Testing Functionality of DHFR Protein

The selection system is based on the fact that bacterial DHFR can be specifically inhibited using trimethoprim, rendering cells dependent upon murine DHFR (mDHFR) activity for their survival. The first test of the system was to establish that mDHFR protein refolds and is active. SDS-PAGE analysis was used to confirm that the protein is highly expressed upon addition of IPTG. Further evidence that the protein is expressed, folds, and is functionally active was verified by transformation of bacterial cells and confirmed by the presence of multiple colonies in minimal media containing trimethoprim.

Establishing an Assay that Uses Cell Survival as a Readout of DNA-Binding Activity

It was next necessary to establish that introduction of an AP-1 component (in this case basic-cJun) would result in binding to the 15 TRE's introduced within the mDHFR gene and therefore failure of the gene to be transcribed.

Three plasmids were used for this assay. These are i) p300-mDHFR (Cm; SEQ ID NO: 42) to express the 12×consensus sequence containing mDHFR, which is under control of the lac-operon; ii) p230d-basic-cJun (Amp; SEQ ID NO: 43) which is also under control of the lac-operon; iii) pREP4 (Kan; SEQ ID NO: 44) to express the lac repressor.

Cells were grown under non-selective conditions (i.e. LB/LB agar) containing Cm/Amp/Kan up until the time of the Assay. During TBS Selection Cells are grown in M9 minimal media (Agar or Broth) in the presence of Cm/Amp/Kan, as well as Tmp (to inhibit the bacterial copies of DHFR) and IPTG (to induce expression of mDHFR and bZIP proteins). During Assay selection, media-lacking ITPG is used to serve as a negative control to ensure that cell survival is exclusively driven by the loss on interaction between bZIP target protein and the consensus sequences located within the mDHFR gene.

As expected, overexpression of basic-cJun on the second plasmid resulted in a complete loss of bacterial colonies in minimal media (FIG. 1B). Without wishing to be bound by theory, it is believe that this works because AP-1 binds to the multiple TREs found within the mDHFR gene and therefore works in the opposite way to its natural function. Rather it works by blocking transcription and preventing the machinery from moving along the DNA. As a control a version of cJun containing the leucine zipper, but lacking in the DNA-binding basic region (SEQ ID NO: 45), was tested. As expected this version did not prevent bacterial colony formation in minimal media.

Discussion

We have shown using the essential enzyme mDHFR that i) enzymatic activity is preserved upon introduction of 15 TREs into the gene under selective conditions activity becomes lost when basic-cJun is introduced, and the basic region within basic-cJun is an absolute requirement for this loss of mDHFR activity. This assay therefore uses cell survival as a marker to allow rapid screening of peptide libraries.

### Example 2—Designing a Cell Assay to Detect Inhibitors to Primary Events in α-Synuclein Aggregation

The assay described in Example 1 demonstrates that an engineered mDHFR gene can be used to detect DNA binding of AP-1 to DNA-binding sites located in the gene and allows for the selection of cells that contain unbound DHFR. It was then proposed to develop a cell-based assay using this mechanism to screen for inhibitors of primary events in α-Synuclein (αS) aggregation.

Generating Components of the Assay

To establish the assay, the DNA-binding basic region of the AP-1 (Activator-protein 1) receptor subunit c-Jun was attached to the N-terminus of αS. AP-1 is a dimeric transcription factor which assembles via a bZIP domain comprising a DNA-binding basic region and a leucine zipper (Seldeen et al. 2009). The AP-1 receptor can comprise a homodimer of Jun proteins or a Jun-Fos heterodimer (Nakabeppu et al. 1988; Sassone-Corsi et al. 1988) and consequently the basic regions of c-Jun were chosen for Basic-αS. The basic region contains positively charged residues for the interaction with TRE sites in DNA containing the conserved motif 5′-TGA G/C TCA-3′. For the assay, the mDHFR reporter plasmid described in Example 1 was used, which contains silent and conserved mutations giving a total of 15 TRE sites.

Firstly, basic-αS DNA was successfully synthesised via PCR. Following the successful amplification and purification of basic-αS, the DNA insert was subcloned into a p230d plasmid. This plasmid was chosen due to its complementarity with the subsequent DHFR(TRE)-p300d plasmid to be used for the assay, both expressing distinct antibiotic resistance. Sequencing confirmed the generation of the p230-basic-αS plasmid.

A pREP4 plasmid was used to encode the lacI gene for regulating expression of mDHFR under the control of the lac operon. IPTG is used to remove the lacI repressor and induce transcription of mDHFR. Under minimal media conditions in which endogenous bacterial DHFR is inhibited, the transcription of mDHFR is essential for cell survival. Therefore, during testing of the assay, negative controls on minimal media lacking IPTG were expected to grow no colonies. Under test conditions with IPTG, mDHFR transcription would yield colonies unless inhibited by other variables.

BL21 E. coli cells harbouring the pREP4 plasmid were co-transformed with DHFR(TRE)-300d and either αSp230d or Basic-αS p230d. Cells were load-matched and plated under three conditions with the overexpression of αS and Basic-αS being the stimulation to aggregate. Positive controls grown on LB agar under selection shows the cells were live and contained all 3 plasmids. M9 minimal agar contained Tmp antibiotic to specifically inhibit the endogenous DHFR protein. Test plates contained IPTG for expression of mDHFR under control of the lac operon. Negative control plates were expected to not produce colonies in the absence of IPTG as pREP4, expressing the lacI repressor, prevents transcription of mDHFR.

Testing the Amyloid-TBS Assay

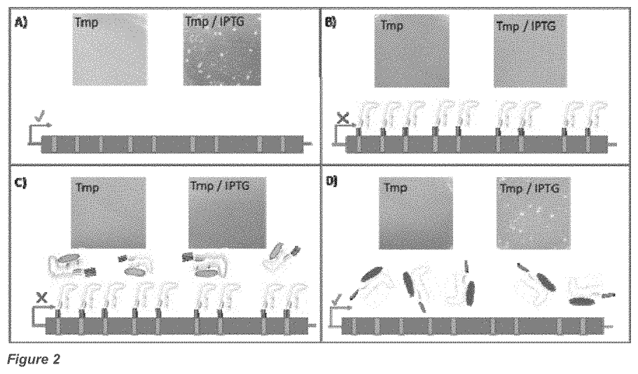

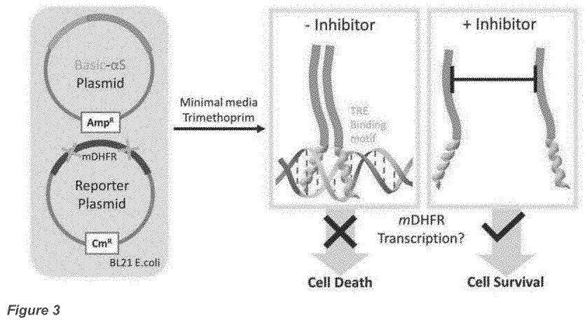

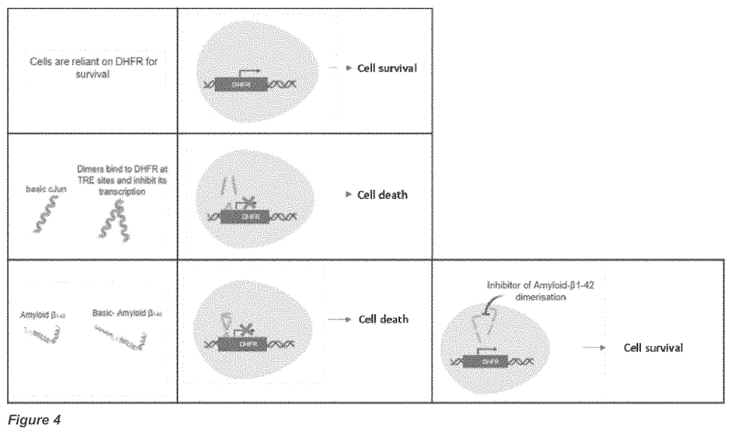

As described above in Example 1, introducing 15 TRE's into the mDHFR gene via silent and conserved mutations resulted in a functional DHFR enzyme, which can be used to maintain cell growth under selective conditions (FIG. 2A). Co-expression of basic-αS leads to occupation of TRE sites on the DHFR gene and blocks transcription of the engineered mDHFR, resulting in cell death (FIG. 2B). As a final proof that this approach is successful, when a peptide (45-54W) designed to bind to αS was co-expressed with the engineered mDHFR and basic-αS cell survival is favoured by the loss of basic-αS DNA binding activity (FIG. 2D). Cell survival was not observed when a control peptide (a scrambled dummy sequence that does not bind αS) was co-expressed with the engineered mDHFR and basic-α (FIG. 2C).

Discussion

αS aggregation forms large, ordered fibrils which are found in Lewy Body inclusions of dopaminergic neurons in patients with Parkinson's Disease (PD). Despite the exact underlying cause for PD being unclear, one strategy for treatment is to prevent αS aggregation. As the disordered nature of αS prevents traditional drug design, this study aimed to develop a new assay for the intracellular screening of αS aggregation inhibitors (FIG. 2). In particular, this assay allows for the selection of inhibitor that block initial dimer formation of αS.

We synthesised basic-αS, which comprises the DNA-binding basic region of c-Jun attached to αS. Normally, c-Jun bind DNA as pairs, with dimerization facilitated by a coiled-coil dimerization domain. However, in the amyloid-TBS assay, dimerization is instead achieved by the αS domains appended to the basic peptide (i.e. no coiled-coil is present in basic-αS). Aggregation of αS causes the basic regions to come in close proximity, as they would in AP-1, to be able to bind TRE sites in a reporter mDHFR plasmid. This prevents mDHFR transcription such that primary events in the aggregation of basic-αS could be detected as a cell death signal when compared to WT αS (FIG. 1B).

In order to establish that the assay could be used to identify inhibitors of the initial αS dimerization event, we made use of 45-54W, a peptide inhibitor that had previously been evaluated as being able to bind αS and reduce aggregation levels at early stages of the misfolding pathway (Cheruvara et al. 2015). It was not conclusive from previous studies whether 45-54W targeted the initial dimerization event. Use of 45-54W restored mDHFR transcription-translation and colony formation, indicating that the inhibitor binds and inhibits initial αS dimerization (FIG. 1D). Importantly, the restoration of colony formation was not observed when a control peptide was used that binds αS but not in the monomeric form (FIG. 1C). Distinguishing between binding and inhibition of dimerization is important since αS binders derived by other means do not necessarily prevent dimer formation (and the subsequent formation of higher-n oligomers, and their conformers). Therefore, such compounds may not translate into functional antagonists of αS pathology.

The TBS-based assay described here allows rapid screening of genetically encoded peptide libraries, to assess cell survival to consequently derive functionally active antagonists of αS pathology. Since peptide libraries are screened entirely inside living cells, the assay described here has the added benefit of removing library members that are toxic, susceptible to proteases, insoluble, or non-specific for αS and detrimental to cell growth. This assay can therefore advantageously be used to select for inhibitors that bind αS, inhibit dimerization and lack cell toxicity in a single step. Furthermore, the assay described here has the significant advantage of concomitantly interrogating exogenously applied peptides for membrane permeability (e.g. naturally, via strand-inducing constraints or CPP appendage), protease resistance, and lack of cytotoxicity.

Following identification of novel inhibitors using the amyloid-TBS assay, biophysical, structural and primary neuron-based cell biology approaches can be used to validate the inhibitor function. For example, the following assays can be used: i) continuous growth ThT experiments, demonstrate inhibition of amyloid formation in a dose-dependent manner ii) single molecule fluorescence and atomic force microscopy (AFM) imaging, confirm prevention of amyloid formation iii) circular dichroism spectroscopy experiments to detect changes in global secondary structure iv)neuronal cell assays to demonstrate reduced αS cytotoxicity v) intracellular delivery of peptides to test colocalization and downstream effects of the in cellulo derived peptides on cytotoxicity and proteostasis in neurons where wild-type or mutant αS is overexpressed. Undertaking these assays using our iterative strategy of Truncation, Randomisation and Selection (TraSe; Crooks et al. 2011) will lead to reduced size antagonists by identifying the smallest functional unit required for effective target binding.

In conclusion, this study shows an intracellular method to identify inhibitors that directly prevent αS aggregation at the initial step in the misfolding pathway. Aggregation of αS underlies the related synucleinopathies, in addition to PD, which means a successful disease-modifying lead could have broad benefits. Finally, with amyloid fibrils underlying other neurodegenerative diseases, the amyloid-TBS assay has the potential to be adapted to study inhibitors of other toxic protein aggregates.

### Example 3—Designing a Cell Assay to Detect Inhibitors of Amyloid β1-42 Dimerization

The strategy described above was also used to design an assay to screen for inhibitors of primary events in Aβ₁₋₄₂ dimerization.

By attaching this basic cJun region to Aβ₁₋₄₂, a functional DNA binder will be created if two constructs dimerize. If introduced to bacteria cells reliant on modified DHFR, basic-Aβ₁₋₄₂ dimerization would lead to blocking of the DHFR gene transcription and so cell death. This provides the basis of a novel cell assay that could be used to find peptide inhibitors, as summarised in the schematic in FIG. 3. Only cells treated with a successful Aβ_{1_42} dimerization inhibitor would produce DHFR and survive, allowing selection for potential AD therapeutics.

Generating a Basic cJun-Aβ₁₋₄₂ (Basic-Aβ₁₋₄₂) Fusion Protein

The DNA binding moiety from cJun is a 25 amino acid coiled coil, made primarily of basic amino acids lysine, arginine and histidine (SEQ ID NO: 47). PCR was used to attach the DNA encoding this basic region (SEQ ID NO: 46) to Aβ₁₋₄₂ DNA (SEQ ID NO: 48) followed by subcloning into a p230d vector. Sanger sequence was performed to confirm that the production of a p230d plasmid containing the basic-Aβ₁₋₄₂ sequence (SEQ ID NO: 50).

Testing the Aβ Dimerization Assay

BL21 GOLD cells stably expressing a pREP4 plasmid containing lac inhibitor (lad) gene were used for the dimerization assay, so that the lac operon could be controlled. Cells were transformed with p300d plasmid containing DHFR with 15 TRE sites under control of the lac operon (described in Example 1), and either a p230d containing Aβ₁₋₄₂ or constructed basic-Aβ₁₋₄₂.

BL21 from the 100 μL plates were scraped into LB and selecting antibiotics and grown to an OD₆₀₀ of 0.5. 100 μL of culture was plated onto positive control, negative control and test plates, as described in Table 3 below.

TABLE 3
Composition of positive, negative and test
plates used in the Aβ₁₋₄₂ dimerization assay

| | Plate type | | |
|---|---|---|---|
| | Positive control | Negative control | Test |
| Media | LB agar | M9 minimal | M9 minimal |
| | | agar | agar |
| Selecting | 100 μM Cm, | 100 μM Cm, | 100 μM Cm, |
| Antibiotics | Kan and Amp | Kan and Amp | Kan and Amp |
| Treatments | — | 3.4 μM Tmp | 3.4 μM Tmp |
| | | (1 mg/mL stock | (1 mg/mL |
| | | in DMSO) | stock in |
| | | | DMSO) |
| | | | 1 mM IPTG |

Results following a 48-hour incubation are shown in Table 4 below. A covering of cells was seen on both positive control plates, showing that BL21 cells were not affected by scraping and re-plating. Tmp was used to inhibit the endogenous, E. coli DHFR enzyme. Both basic-Aβ₁₋₄₂ and Aβ₁₋₄₂ expressing cells were shown to be reliant on the modified DHFR because limited growth was seen on M9 minimal media plates, consistent with the lac repressor protein from pREF4 preventing expression of modified DHFR and resulting in a lack of THFA. Two basic-Aβ₁₋₄₂ expressing colonies and one Aβ₁₋₄₂ colony grew on these negative control plates. Test plates contained IPTG to bind to lac repressor protein and allow expression of the modified DHFR in the bacteria. There were 63 Aβ₁₋₄₂ expressing BL21 colonies formed on these plates, which confirmed that the modified DHFR gene allows cells to grow on minimal media. Importantly, only 2 basic-Aβ₁₋₄₂ expressing colonies grew on test plates, which was consistent with levels of background growth seen on negative control plates. This indicates that basic-Aβ₁₋₄₂ protein dimerized and blocked modified DHFR leading to cell death. These results support the use of these cells in a basic-Aβ₁₋₄₂ dimerization assay with cells only surviving if inhibition is achieved.

TABLE 4
Colony growth in basic-Aβ1-42 dimerization assay

| | | No. of colonies (CFU) | |
|---|---|---|---|
| | | | basic-Aβ₁₋₄₂ |
| | | Aβ₁₋₄₂ expressing | expressing |
| Media type | Positive control | Covered | Covered |
| | Negative control | 1 | 2 |
| | Test | 63 | 2 |

Discussion

Inhibition of Aβ₁₋₄₂ dimerization prevents formation of all types of Aβ₁₋₄₂ oligomers making it an attractive therapeutic approach for AD. To screen for peptide inhibitors of this event, an in-cell detection assay was proposed using E. coli BL21 GOLD cells reliant on a modified DHFR containing cJun binding sites. The DNA binding moiety from cJun was successfully cloned onto the N-terminus of Aβ₁₋₄₂ such that when expressed in these BL21 cells, the dimerized protein blocked DHFR transcription and lead to cell death.

Cloned p230d-basic-Aβ₁₋₄₂ DNA was used to create an A431-42 dimerization assay. BL21 GOLD cells containing pREF4 were used for their protein expression ability and for the lacI gene to control the lac operon. Cells were transformed with p300d encoding modified DHFR DNA, and either p230d-basic-A431-42 or p230d-Aβ₁₋₄₂. Results in Table 4 showed that on M9 minimal media, the lac repressor from pREF4 prevented expression of modified DHFR and resulted in a lack of THFA and so cell death. Two basic-Aβ₁₋₄₂ expressing colonies and one Aβ₁₋₄₂ colony grew on these negative control plates, perhaps due to insufficient exposure to Tmp, and so a higher concentration than 3.5 μM could be used if repeated to ensure all endogenous DHFR was inhibited. In the presence of IPTG, modified DHFR was expressed in the bacteria allowing for survival of Aβ₁₋₄₂ expressing BL21 colonies (Table 4). Only 2 basic-Aβ₁₋₄₂ expressing colonies survived in these conditions, indicating basic-Aβ₁₋₄₂ protein dimerized and blocked modified DHFR as expected. This gives confidence that basic-Aβ₁₋₄₂ expression can be used as a screening assay, with cells surviving if inhibition is achieved.

This BL21 GOLD assay system benefits from a survival endpoint making it possible to easily screen large libraries of peptides for dimerization inhibitors. The library of peptides could perhaps be designed using the dimerization interface of Aβ itself as a starting point as it is a self-dimer. Future experiments could demonstrate proof of principle using known inhibitors of amyloidosis such as the beta sheet breaker iAβ5 (Adessi and Soto, 2002) in order to show the expected level of cell growth of a successful inhibitor.

Peptides that inhibit the dimerization of basic cJun might be found in the assay which would not be useful for AD drug discovery. To overcome this, follow-up biophysical experiments monitoring peptide binding to wild-type Aβ₁₋₄₂ protein could be used. For example, Surface Plasma Resonance or Isothermal Calorimetry could be performed to detect protein binding of peptides. X-ray crystallography of Aβ₁₋₄₂ crystals soaked in potential inhibitors would also provide information about the mechanism of action of the inhibitor. Because the assay is performed in bacteria and not a disease relevant cortical neuron cell line, inhibitors will also require further testing to ensure the same effect is seen when in neurons or in vivo AD models. However, the assay does detect dimerization of human Aβ₁₋₄₂ and so is useful for initial screening to finding peptides that can disrupt this.

In conclusion, expression of basic-Aβ₁₋₄₂ in bacteria reliant on modified DHFR has created a novel system that detects Aβ₁₋₄₂ dimerization.

### Example 4—Library Creation

Dimerization Assay—Genetically Encoded Library Construction:

As described above, three plasmids were used for the dimerization assays. These are i) p300-mDHFR (Cm) to express the mDHFR containing 15 AP-1 binding sites, which is under control of the lac-operon; ii) p230d-basic-cJun fusion protein (basic-Aβ₁₋₄₂ or basic-αS; Amp) which is also under control of the lac-operon; iii) pREP4 (Kan) to express the lac repressor.

Genetically encoded libraries are created using overlap extension PCR, subcloned into the p410d vector (Tet) and plated out. Each colony then represents a member of the library. We typically collect 2-5× the library size in colony numbers to gain approx. 95% total coverage. The maximum library size screenable using the approach is 10⁷. Once the library is complete colonies are pooled and mini-preparation of DNA performed. Finally the plasmid library is transformed into cells containing p300/p230/pREP4. During single step selection cells are plated onto LB agar (to demonstrate successful transformation), M9 agar lacking IPTG (as a negative control where no bZIP or mDHFR is expressed) and finally onto M9 agar containing Cm/Amp/Kan/Tet/Tmp/IPTG to drive production of basic-cJun fusion protein/mDHFR/Library such that cell viability is only restored if a given library member can prevent the cJun target from interacting with the cognate sequences within the mDHFR gene. Surviving colonies can next be pooled, grown and serially diluted in liquid cultures under selective conditions (M9 minimal medium with 1 μg/ml trimethoprim). Fastest growth, and hence the highest affinity interacting partners dominated the pool. Library pools as well as colonies from individual clones were sequenced to verify the arrival at one sequence. To assess library quality we sequence pools and single clones to find approximately equal distributions of varied amino acids. Pooled colonies exceeded the library size 5-10 fold. Using more recent ligation methods (Topo/Gibson/Gateway) it may be possible to move into the dimerization assay directly from ligation, giving the significant advantage of being able to screen larger libraries (possibly up to 10¹⁰ or 10¹¹), however processes will need to be put into place (e.g. next gen sequencing) to ensure that library size and quality is fully represented prior to transformation into the dimerization assay.

Another possibility is to use pET24a as an alternative to the pREP4 vector used to express the lac repressor. This would allow the expression of both the lac repressor and library/antagonist off a single plasmid, i.e. avoiding the need for another antibiotic.

Dimerization Assay—Extracellular Compound Addition:

For extracellular libraries, cells containing p300-mDHFR plasmid are grown in the presence of p230d-basic-cJun fusion protein and pREP4 plasmids under non-selective conditions (LB agar/media). Once ready for assay overnights can then be placed into each well of microtitre plates (96, 384, 1536) at A600=0.05 and compound libraries screened by direct addition to each well. Plates are incubated at 37° C. and with shaking and successful compounds identified by monitoring of the absorbance signal at 600 nm. This extracellular compound addition method has the advantage of allowing the user to move away from standard peptide libraries (e.g. one can profile for helix constrained peptides, peptidomimetics, non-natural amino acids etc., or even small molecule libraries) and importantly allows the user to profile for cell penetrance concomitantly with the ability to inhibit dimerization. Once again, all proteins are under control of a lac promoter, and expression was induced with Isopropyl β-D-1-thiogalactopyranoside (IPTG).

Selection of Winner Peptides

Briefly, during the assay peptides (intracellular) or compounds (extracellular) that can disrupt dimerization of the basic-cJun fusion proteins will result in colony formation/cell growth on M9 minimal medium plates/media with 1 μg/ml trimethoprim to inhibit bacterial DHFR.

### Example 5—Introducing the CRE, CCAAT and Ebox Binding Sites into the DHFR Gene

Constructs were designed whereby the CRE, CCAAT and Ebox binding sites, respectively, were inserted into the DHFR gene. These constructs can be tested in the assays described above.

Inserting the CRE Binding Site into the DHFR Gene

CRE is usually defined as TGACGTCA (SEQ ID NO: 10). Mutations in the DHFR gene can be made by inspection of the desired consensus sequence and all three frames and the corresponding changes to the amino acid sequence upon making the necessary single base-pair changes. The CRE is 8 bp as so can span four codons. For example, the sequence defined above can be put into any one of the three reading frames and the corresponding amino acid sequence and tolerated variations can be given:

A: Frame 1: TGA CGT CAx 1 = stop 2:R 3:H/Q
B: Frame 2: xTG ACG TCA 1:LMV 2:T 3:S
C: Frame 3: xxT GAC GTC Axx 1:FLIVSPTAYHNDCRG 2:D 3:V 4:IMTNKSR

From this it is possible to implement changes into the mDHFR gene to give minimal perturbation to the overall sequence. Mutations should be placed at solvent exposed sites and away from the catalytic centre and where possible mutations should be silent or conservative.

An example of an mDHFR gene that is modified to contain CRE binding sites is shown as follows:

ATGGTTCGACCATTGAACTGCATCGTCGCCGTGTCCCAAAATATGGGGATTGGCAAGAACGGAGACCTAC
CCTGGCCTCCGCTCAGGAACGAGTTCAAGTACTTCCAAAGAATGACGTCAACCTCTTCAGTGGAAGGTAA
ACAGAATCTGGTGATTATGGGTAGGAAAACCTGGTTCTCCATTCCTGAGAAGAATCGACCTTTAAAGGAC
AGAATTAATATAGTGACGTCAAGAGAACTCAAAGAACCACCACGAGGAGCTCATTTTCTTGCCAAAAGTT
TGGATGATGCCTTAAGACTTATTGAACAACCGGAATTGACGTCAAAAGTAGACATGGTTTGGATCGTCGG
AGGCAGTTCTGTTTACCAGGAAGCCATGAATCAACCAGGCCACCTTAGACTCTTTGTGACGTCAATCATG
CAGGAATTTGAAAGTGACACGTTTTTCCCAGAAATTGATTTGGGGAAATATAAACTTCTCCCAGAATACC
CAGGCGTGACGTCAGAGGTCCAGGAGGAAAAAGGCATCAAGTATAAGTTTGAAGTCTACGAGAAGAAAGA
CTAAGCTTAA

Nucleotide residues in bold underline indicate consensus CRE binding sites.

Nucleotide residues in lowercase and italics correspond to the restriction enzyme sites for AscI and HindIII at the 5′ and 3′ ends of the sequence, respectively.

The resulting amino acid sequence is shows as follows:

M V R P L N C I V A V S Q N M G I G K N
G D L P W P P L R N E F K Y F Q R M T [symbol]
T S S V E G K Q N L V I M G R K T W F S
I P E K N R P L K D R I R I V [symbol] S R E L
K E P P R G A H F L A K S L D D A L R L
I E Q P E L [symbol] S K V D M V W I V G G S S
V Y Q E A M N Q P G H L R L F V T [symbol] I M
Q E F E S D T F F P E I D L G K Y K L L
P E Y P G V [symbol] S E V Q E E K G I K E K F
E V Y E K K D

Amino acid residues in italics are solvent exposed residues. The other residues are classed as buried residues.

Amino acid residues in bold underline are residues that have been altered as a result of the insertion of CRE into the nucleotide sequence.

A summary of the amino acid changes is provided as follows:

1. MTT→MTS (T40S) = ATG ACG TCA ASA at posn = 36
2. VLS→VTS (L76T) = GTG ACG TCA ASA at posn = 21
3. LAS→LTS (A107T) = TTG ACG TCA ASA at posn = 37
4. VTR→VTS (R138S) = GTG ACG TCA ASA at posn = 57
5. VLS→VTS (L167T) = GTG ACG TCA ASA at posn = 99

Inserting the CCAAT Binding Site into the DHFR Gene

CCAAT is usually defined as ATTGCGCAAT (SEQ ID NO: 9). Mutations in the DHFR gene can be made by inspection of the desired consensus sequence and all three frames and the corresponding changes to the amino acid sequence upon making the necessary single base-pair changes. The CCAAT is 10 bp and so can span five codons. For example, the sequence defined above can be put into any one of the three reading frames and the corresponding amino acid sequence and tolerated variations can be given:

A:Frame 1: ATT GCG CAA Txx 1:1 2:A 3:Q 4:FLSYCW
B:Frame 2: xAT TGC GCA ATx 1:YHND 2:C 3:A 4:IM
C:Frame 3: xxA TTG CGC AAT 1:LIVSPTAQKERG* 2:L 3:R 4:N

From this it is possible to implement changes into the mDHFR gene to give minimal perturbation to the overall sequence. Mutations should be placed at solvent exposed sites and away from the catalytic centre and where possible mutations should silent or conservative.

An example of an mDHFR gene that is modified to contain CCAAT is shown as follows:

ATGGTTCGACCATTGAACTGCATCGTCGCCGTGTCCCAAAATATGGGGATTGGCAAGAACGGAGACCTAC
CCTGGCCTCCATTGCGCAATGAGTTCAAGTACTTCCAAAGAATGACCACAACCTCTTCAGTGGAAGGTAA
ACAGAATCTGGTGATTATGGGTAGGAAAACCTGGTTCTCCATTCCTGAGAAGAATCGACCATTGCGCAAT
AGAATTAATATAGTTCTCAGTAGAGAATTGCGCAATCCACCACGAGGAGCTCATTTTATTGCGCAATCCT
TGGATGATGCATTGCGCAATATTGAACAACCGGAATTGGCGAGCAAAGTAGACATGGTTTGGATCGTCGG
AGGCAGTTCTGTTTACCAGGAAGCCATGAATCAACCAGGCCACCTTAGACTCTTTGTGACAAGGATCATG
CAGGAATTTGAAAGTGACACGTTTTTCCCAGAAATTGATTTGGGGAAATATAAACTTCTCCCAGAATACC
CAGGCGTCCTCTCTGAATTGCGCAATGAAAAAGGCATCAAGTATAAGTTTGAAGTCTACGAGAAGAAAGA
CTAAGCTTAA

Nucleotide residues in bold underline indicate CCAAT consensus binding sites.

Nucleotide residues in lowercase and italics correspond to the restriction enzyme sites for AscI and HindIII at the 5′ and 3′ ends of the sequence, respectively.

The resulting amino acid sequence is shows as follows:

M V R P L N C I V A V S Q N M G I G K N
G D L P W P P L R N E F K Y F Q R M T T
T S S V E G K Q N L V I M G R K T W F S
I P E K N R P L [symbol] R I N I V L S R E L
P P R G A H F [symbol] A [symbol] S L D D A L R [symbol]
I E Q P E L A S K V D M V W I V G G S S
V Y Q E A M N Q P G H L R L F V T R I M
Q E F E S D T F F P E I D L G K Y K L L
P E Y P G V L S E V Q E E K G I K Y K F
E V Y E K K D

Amino acid residues in italics are solvent exposed residues. The other residues are classed as buried residues.

Amino acid residues in bold underline are residues that have been altered as a result of the insertion of CCAAT into the nucleotide sequence.

A summary of the amino acid changes is provided as follows:

1. PLRN (silent)
2. PLKD→PLRN (K69R, D70N) = ASA at posns = 90, 97
3. ELKE→ELRN (K81R, E82N) = ASA at posns = 175, 131
4. LAKS→IAQS (L90I, K92Q) = ASA at posns = 47, 139
5. ALRL→ALRN (L100N) = ASA at posn = 46

A further CCAAT site could be inserted to make the following mutation:

6. EVQE→ELRN (V170L, Q171R, E172N)

Inserting the Ebox Binding Site into the DHFR Gene

In the context of cMyc, Ebox is usually defined as CACGTG (SEQ ID NO: 7) or CACATG (SEQ ID NO: 8). Mutations in the DHFR gene can be made by inspection of the desired consensus sequence and all three frames and the corresponding changes to the amino acid sequence upon making the necessary single base-pair changes. For example, the sequences defined above can be put into any one of the three reading frames and the corresponding amino acid sequence and tolerated variations can be given:

A:Frame 1: CAC GTG XXX = 1:H 2:V 3:Anything
B:Frame 2: xCA CGT Gxx = 1:S/P/T/A 2:R 3:V/A/D/E/G
C:Frame 3: xxC ACG TGx = 1:FLIVSPTAYHNDCRSG 2:T 3:C/W/*
A:Frame 1: CAC ATG xxx = 1:H 2:M 3:anything
B:Frame 2: xCA CAT Gxx = 1:S/P/T/A 2:H 3:V/A/D/E/G
C:Frame 3: xxC ACA TGx = 1:FLIVSPTAYHNDCRSG 2:T 3:C/W/*

From this it is possible to implement changes into the mDHFR gene to give minimal perturbation to the overall sequence. Mutations should be placed at solvent exposed sites and away from the catalytic centre and where possible mutations should silent or conservative.

An example of an mDHFR gene that is modified to contain Eboxes is shown as follows:

ATGGTTCGACCATTGAACTGCATCGTCGCCGTGTCCCAAAATATGGGGATTGGCAAGAACGGAGACCTAC
CCTGGCCTCCGCTCAGGAACGAGTTCAAGTACTTCCAAAGAATGACCACAACCTCTTCAGTGGAAGGTAA
ACAGAATCTGGTGATTATGGGTAGGCGCACGTGGTTCTCCATTCCTGAGAAGAATCGACCTTTAAAGGAC
AGAATTAATATAGTTCTCTCACGTGAACTCAAAGAACCACCACGTGGAGCTCACGTGCTTGCCAAATCAC
TGGATGATGCATTAAGACTTATTGAACAACCGGAATTGGCGTCACGTGTAGACATGGTTTGGATCGTCGG
AGGCAGTTCTGTTTACCAGGAAGCCATGAATCAACCAGGCCACGTGAGACTCTTTGTGACACGTGTCATG
CAGGAATTTGAAAGTGACACGTTTTTCCCAGAAATTGATTTGGGGAAATATAAACTTCTCCCAGAATACC
CAGGCGTCCTCTCACGTGTCCAGGAGGAAAAAGGCATCAAGTATAAGTTTGAAGTCTACGAGAAGAAAGA
CTAAGCTTAA

Nucleotide residues in bold underline indicate Ebox consensus binding sites.

Nucleotide residues in lowercase and italics correspond to the restriction enzyme sites for AscI and HindIII at the 5′ and 3′ ends of the sequence, respectively.

The resulting amino acid sequence is shows as follows:

M V R P L N C I V A V S Q N M G I G K N
G D L P W P P L R N E F K Y F Q R M T T
T S S V E G K Q N L V I M G R [symbol] T W F S
I P E K N R P L K D R I N I V L S R E L
K E P P R G A H [symbol] L A K S L D D A L R L
I E Q P E L A S [symbol] V D M V W I V G G S S
V Y Q E A M N Q P G H [symbol] R L F V T R [symbol] M
Q E F E S D T F F P E I D L G K Y K L L
P E Y P G V L S [symbol] V Q E E K G I K Y K F
E V Y E K K D

Amino acid residues in italics are solvent exposed residues. The other residues are classed as buried residues.

Amino acid residues in bold underline are residues that have been altered as a result of the insertion of Ebox into the nucleotide sequence.

A summary of the amino acid changes is provided as follows:

1. KTW→RTW (K56R) = CGC ACG TGG ASA at posn = 143 (exposed)
2. SRE (silent) = TCA CGT GAA ASA at posn = N/A
3. PRG (silent) = CCA CGT GGA ASA at posn = N/A
4. HFL→HVL (F89V) = CAC GTG CTT ASA at posn = 71 (exposed)
5. SKV→SRV (K109R) = ACA CGT GTA ASA at posn = 109 (exposed)
6. HLR→HVR (L132V) = CAC GTG AGA ASA at posn = 1.6
7. TRI→TRV (1139V) = ACA CGT GTC ASA at posn =1.6
8. SEV→SRV (E151R) = ACA CGT GTC ASA at posn = 141 (exposed)

Changes 6 and 7 are located at residues that are classed as buried. Accordingly, constructs could be made that contain all 8 Ebox sites, one that is lacking site ‘6’, one that is lacking site ‘7’ and one that is lacking both sites ‘6’ and ‘7’ in order to determine whether the mutation at these ‘buried’ sites affect the function of the resultant DHFR protein.

### REFERENCES

A number of publications are cited above in order to more fully describe and disclose the invention and the state of the art to which the invention pertains. Full citations for these references are provided below. The entirety of each of these references is incorporated herein.

### REFERENCES

Altschul, G. F. et al. (1990) Basic local alignment search tool. J Mol Biol., 215(3):403-10.
Andrew, R. J. et al. (2016) A Greek Tragedy: The Growing Complexity of Alzheimer Amyloid Precursor Protein Proteolysis. J Biol Chem, 291, pp. 19235-18244.
Arosio, P et al. (2015). On the lag phase in amyloid fibril formation. Physical Chemistry Chemical Physics, 17(12), pp. 7606-7618.
Berriman J. et al. (2003) Tau filaments from human brain and from in vitro assembly of recombinant protein show cross-β structure. Proc. Natl. Acad. Sci. U.S.A., 100(15): 9034-9038
Burre, J et al. (2010) a-Synuclein Promotes SNARE-Complex Assembly in Vivo and in Vitro. Science, 329(5999), pp. 1663-1667.
Cabezas, E.; Satterthwait, A. C. (1999) J. Am. Chem. Soc., 121, 3862.
Chiti, F.; Dobson, C. (2017) Protein Misfolding, Amyloid Formation, and Human Disease: A Summary of Progress Over the Last Decade Annu. Rev. Biochem. 86:27-68
Cheruvara, et al. Intracellular Screening of a Peptide Library to Derive a Potent Peptide Inhibitor of alpha-Synuclein Aggregation. J. Biol. Chem. 2015, 290 (12), 7426-35.
Cody, V. et al. (2006) New insights into DHFR interactions: Analysis of Pneumocystis carinii and mouse DHFR complexes with NADPH and two highly potent 5-(omega-carboxy(alkyloxy) trimethoprim derivatives reveals conformational correlations with activity and novel parallel ring stacking interactions. Proteins 65(4): 959-969
Crooks, R. O. et al. (2011) Generation of a Reduced Length c-Jun Antagonist That Retains High Interaction Stability. J. Biol. Chem. 286 (34), 29470-9.
de Araujo A.D. et al. (2014) Comparative a-helicity of cyclic pentapeptides in water. Angew Chem Int Ed Engl. 53(27):6965-9
Fauvet, B. et al. (2012) α-Synuclein in Central Nervous System and from Erythrocytes, Mammalian Cells, and Escherichia coli Exists Predominantly as Disordered Monomer. Journal of Biological Chemistry, 287(19), pp. 15345-15364.
Fujimoto, K. et al. (2008) Development of a series of cross-linking agents that effectively stabilize alpha-helical structures in various short peptides. Chemistry 14(3):857-63.
Gremer, L. et al. (2017). Fibril structure of amyloid-β(1-42) by cryo-electron microscopy. Science, 358, pp. 116-119.
Haney, C. M. et al. (2011) Promoting peptide α-helix formation with dynamic covalent oxime side-chain cross-links. Chem Commun (Camb).47(39):10915-7.
Holland-Nell, K.; Meldal, M. Maintaining biological activity by using triazoles as disulfide bond mimetics. Angew Chem Int Ed Engl. 50(22):5204-6.
Jakes, R. et al. (1994) Identification of two distinct synucleins from human brain. FEBS Letters, 345(1), pp. 27-32.
Jao, C. C. et al. (2004). From The Cover: Structure of membrane-bound a-synuclein studied by site-directed spin labeling. Proceedings of the National Academy of Sciences, 101(22), pp. 8331-8336.
Jo, H. et al. (2012) Development of α-helical calpain probes by mimicking a natural protein-protein interaction. J Am Chem Soc. 134(42):17704-17713
Kaufman et al. (1986) Selection and amplification of heterologous genes encoding adenosine deaminase in mammalian cells. Proc Natl Acad Sci USA. 83(10): 3136-3140.
Kim et al. (2006) A high-throughput screen for compounds that inhibit aggregation of the Alzheimer's peptide. ACS Chem Biol. 1(7):461-9.
Kurnik, M. et al. (2018). Potent α-Synuclein Aggregation Inhibitors, Identified by High-Throughput Screening, Mainly Target the Monomeric State. Cell Chemical Biology, 25(11), p. 1389-1402.e9.
Lashuel, H. A. et al. (2002). Amyloid pores from pathogenic mutations. Nature, 418(6895), pp. 291-291.
Leduc, A. M. et al. (2003) Helix-stabilized cyclic peptides as selective inhibitors of steroid receptor-coactivator interactions. Proc Natl Acad Sci USA. 100(20):11273-8
Ma. Y. et al (2014) Split focal adhesion kinase for probing protein-protein interactions. Biochemical Engineering Journal. 90: 272-278
Masters, C. L. et al. (2011) Overview and recent advances in neuropathology. Part 2: Neurodegeneration. Pathology, 43(2), pp. 93-102.
Mern, D. S. et al. (2010) Inhibition of Id proteins by a peptide aptamer induces cell-cycle arrest and apoptosis in ovarian cancer cells. Br J Cancer. 103(8): 1237-1244.
Muppidi, A. et al. (2011) Achieving cell penetration with distance-matching cysteine cross-linkers: a facile route to cell-permeable peptide dual inhibitors of Mdm2/Mdmx. Chem Commun (Camb). 47(33):9396-8. Nakabeppu, Y. et al. (1988) DNA binding activities of three murine Jun proteins: Stimulation by Fos. Cell, 55(5), pp. 907-915.
Newman & Keating (2003) Comprehensive identification of human bZIP interactions with coiled-coil arrays. Science. 300(5628):2097-101
Park, J. H. (2007) Bacterial beta-lactamase fragmentation complementation strategy can be used as a method for identifying interacting protein pairs. Journal of Microbiology and Biotechnology. 17 (10): 1607-15.
Pelay-Gimeno, M. et al. (2015) Structure-Based Design of Inhibitors of Protein-Protein Interactions: Mimicking Peptide Binding Epitopes. Angew Chem Int Ed Engl. 54(31):8896-927
Pospich, S. and Raunser, S., (2017). The molecular basis of Alzheimer's plaques. Science, 358, pp. 45-46.
Remy, I. et al. (2007) Detection of protein-protein interactions using a simple survival protein-fragment complementation assay based on the enzyme dihydrofolate reductase. Nat Protoc. 2(9): 2120-5. Rodriguez-Martinez et al. (2017). Combinatorial bZIP dimers display complex DNA-binding specificity landscapes. Elife. 6 e19272
Ruan, F. et al. (1990) Metal ion-enhanced helicity in synthetic peptides containing unnatural, metal-ligating residues J. Am. Chem. Soc., 112 (25): 9403-9404
Sassone-Corsi, P. et al. (1988). fos-associated cellular p39 is related to nuclear transcription factor AP-1. Cell, 54(4), pp. 553-560.
Seldeen, K. L. et al. (2009) Single Nucleotide Variants of the TGACTCA Motif Modulate Energetics and Orientation of Binding of the Jun-Fos Heterodimeric Transcription Factor. Biochemistry, 48(9), pp. 1975-1983.
Takami, M. et al., (2009). γ-Secretase: Successive Tripeptide and Tetrapeptide Release from the Transmembrane Domain of β-Carboxyl Terminal Fragment. The Journal of Neuroscience, 29, pp. 13042-13052.
Ulmer, T. S. et al. (2005) Structure and Dynamics of Micelle-bound Human a-Synuclein. Journal of Biological Chemistry, 280(10), pp. 9595-9603.
Vaquerizas, J. M. et al. (2009) A census of human transcription factors: function, expression and evolution. Nat Rev Genet. 10(4): 252-63
Vinson, C. et al. (2002) Classification of human B-ZIP proteins based on dimerization properties. Mol Cell BioL 22(18):6321-35.
Walensky, L. D. et al. (2004) Activation of apoptosis in vivo by a hydrocarbon-stapled BH3 helix. Science. 305(5689):1466-70.
Wang, D. et al. (2005) Enhanced metabolic stability and protein-binding properties of artificial alpha helices derived from a hydrogen-bond surrogate: application to Bcl-xL. Angew Chem Int Ed Engl. 44(40):6525-9.
Wehr, M. C. et al. (2006) Monitoring regulated protein-protein interactions using split TEV. Nat Methods. 3(12):985-93.
Woolley, G. A. (2005) Photocontrolling peptide alpha helices. Acc Chem Res; 38(6):486-93.

For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press

Sequence Annex

Amino acid sequence of wild-type murine dihydrofolate reductase (SEQ ID NO: 1)
MVRPLNCIVAVSQNMGIGKNGDLPWPPLRNEFKYFQRMTTTSSVEGKQNLVIMGRKTWFSIPEKNRPLKD
RINIVLSRELKEPPRGAHFLAKSLDDALRLIEQPELASKVDMVWIVGGSSVYQEAMNQPGHLRLFVTRIM
QEFESDTFFPEIDLGKYKLLPEYPGVLSEVQEEKGIKYKFEVYEKKD
Amino acid sequence of engineered murine dihydrofolate reductase (SEQ ID NO: 2)
MVRPLNCIVAVSQNMGIGKNGDLPWPPLRNESKYFQRMTQTDSVESKQNLVIMGRKTWFSIPESNRPLKD
RINIVLSQELKEPPRGAHFLAKSLDDALRLIESPELASKVDSVWIVGGSSVYQEAMTQPGHLRLFVTQIM
QEFESDTFFPEIDSGKYKLLPESPGVLSQVQEEKGIKYKFEVYEKKD
Amino acid sequence of wild-type human dihydrofolate reductase (SEQ ID NO: 3)
MVGSLNCIVAVSQNMGIGKNGDLPWPPLRNEFRYFQRMTTTSSVEGKQNLVIMGKKTWFSIPEKNRPLKG
RINLVLSRELKEPPQGAHFLSRSLDDALKLTEQPELANKVDMVWIVGGSSVYKEAMNHPGHLKLFVTRIM
QDFESDTFFPEIDLEKYKLLPEYPGVLSDVQEEKGIKYKFEVYEKND
Nucleic acid sequence for the protein coding sequence of engineered murine dihydrofolate
reductase (SEQ ID NO: 4)
ATGGTTCGACCATTGAACTGCATCGTCGCCGTGAGTCAGAATATGGGGATTGGCAAGAACGGAGACCTACCCTGGCC
TCCGCTCAGGAATGAGTCAAAGTACTTCCAAAGAATGACTCAGACTGACTCAGTTGAGTCAAAACAGAATCTGGTGA
TTATGGGTAGGAAAACCTGGTTCTCCATTCCTGAGTCAAATCGACCTTTAAAGGACAGAATTAATATAGTTCTGAGT
CAAGAACTCAAAGAACCACCACGAGGAGCTCATTTTCTTGCCAAAAGTTTGGATGATGCCTTAAGACTTATTGAGTC
ACCGGAATTGGCGAGCAAAGTTGACTCAGTTTGGATCGTCGGAGGCAGTTCTGTTTACCAGGAAGCCATGACTCAAC
CAGGCCACCTTAGACTCTTTGTGACTCAGATCATGCAGGAATTTGAGTCAGACACGTTTTTCCCAGAAATTGACTCA
GGGAAATATAAACTTCTCCCTGAGTCACCAGGCGTCCTGAGTCAGGTCCAGGAGGAAAAAGGCATCAAGTATAAGTT
TGAAGTCTACGAGAAGAAAGACTAA
Nucleic acid seguences of TPA response elements (TRE)
tgactca (SEQ ID NO: 5)
tgagtca (SEQ ID NO: 6)
Nucleic acid seguences of Ebox response elements
cacgtg (SEQ ID NO: 7)
CACATG (SEQ ID NO: 8)
Nucleic acid seguence of C/EBP protein response element
ATTGCGCAAT (SEQ ID NO: 9)
Nucleic acid seguence of cAMP response element (CRE)
TGACGTCA (SEQ ID NO: 10)
Nucleic acid seguences of Maf recognition elements (MAREs)
TGCTGA^{G}/_{C}TCAGCA (SEQ ID NO: 32)
tgctga^{GC}/_{CG}TCAGCA (SEQ ID NO: 33)
Nucleic acid seauence of Par/CREB-2/PAP binding site
TTACGTAA(SEQ ID NO: 34)
Nucleic acid seauence of polynucleotide encodina enaineered murine dihvdrofolate reductase
including restriction enzyme sites (SEQ ID NO: 11)
GCTAGCGTTCGACCATTGAACTGCATCGTCGCCGTGAGTCAGAATATGGGGATTGGCAAGAACGGAGACCTACCCTG
GCCTCCGCTCAGGAATGAGTCAAAGTACTTCCAAAGAATGACTCAGACTGACTCAGTTGAGTCAAAACAGAATCTGG
TGATTATGGGTAGGAAAACCTGGTTCTCCATTCCTGAGTCAAATCGACCTTTAAAGGACAGAATTAATATAGTTCTG
AGTCAAGAACTCAAAGAACCACCACGAGGAGCTCATTTTCTTGCCAAAAGTTTGGATGATGCCTTAAGACTTATTGA
GTCACCGGAATTGGCGAGCAAAGTTGACTCAGTTTGGATCGTCGGAGGCAGTTCTGTTTACCAGGAAGCCATGACTC
AACCAGGCCACCTTAGACTCTTTGTGACTCAGATCATGCAGGAATTTGAGTCAGACACGTTTTTCCCAGAAATTGAC
TCAGGGAAATATAAACTTCTCCCTGAGTCACCAGGCGTCCTGAGTCAGGTCCAGGAGGAAAAAGGCATCAAGTATAA
GTTTGAAGTCTACGAGAAGAAAGACTAA
Nucleic acid sequences of example reading frames
Example reading frame 1: tga ctc Axx (SEQ ID NO: 12)
Example reading frame 2: xTG act cax (SEQ ID NO: 13)
Example reading frame 3: xxT gac tca (SEQ ID NO: 14)
Amino acid sequence of example reading frame 3 (SEQ ID NO: 15)
FSYCLPHRITNVADG
Amino acid seguence of example codon triplets containing TREs
GTGAGTCAG (SEQ ID NO: 16)
AATGAGTCA (SEQ ID NO: 17)
ATGACTCAG (SEQ ID NO: 18)
ACTGACTCA (SEQ ID NO: 19)
GTTGAGTCA (SEQ ID NO: 20)
CCTGAGTCA (SEQ ID NO: 21)
CTGAGTCAA (SEQ ID NO: 22)
ATTGAGTCA (SEQ ID NO: 23)
GTTGACTCA (SEQ ID NO: 24)
ATGACTCAA (SEQ ID NO: 25)
GTGACTCAG (SEQ ID NO: 26)
TTTGAGTCA (SEQ ID NO: 27)
ATTGACTCA (SEQ ID NO: 28)
CCTGAGTCA (SEQ ID NO: 29)
CTGAGTCAG (SEQ ID NO: 30)
Amino acid seguence of engineered murine dihvdrofolate reductase used during design process
(SEQ ID NO: 31)
* = stop codon
ASVRPLNCIVAVSQNMGIGKNGDLPWPPLRNESKYFQRMTQTDSVESKQNLVIMGRKTWFSIPESNRPLK
DRINIVLSQELKEPPRGAHFLAKSLDDALRLIESPELASKVDSVWIVGGSSVYQEAMTQPGHLRLFVTQI
MQEFESDTFFPEIDSGKYKLLPESPGVLSQVQEEKGIKYKFEVYEKKD*A*
Nucleotide sequence of an exemplary murine dihydrofolate reductase gene engineered to
include CRE binding sites (SEQ ID NO: 36)
ATGGTTCGACCATTGAACTGCATCGTCGCCGTGTCCCAAAATATGGGGATTGGCAAGAACGGAGACCTAC
CCTGGCCTCCGCTCAGGAACGAGTTCAAGTACTTCCAAAGAATGACGTCAACCTCTTCAGTGGAAGGTAA
ACAGAATCTGGTGATTATGGGTAGGAAAACCTGGTTCTCCATTCCTGAGAAGAATCGACCTTTAAAGGAC
AGAATTAATATAGTGACGTCAAGAGAACTCAAAGAACCACCACGAGGAGCTCATTTTCTTGCCAAAAGTT
TGGATGATGCCTTAAGACTTATTGAACAACCGGAATTGACGTCAAAAGTAGACATGGTTTGGATCGTCGG
AGGCAGTTCTGTTTACCAGGAAGCCATGAATCAACCAGGCCACCTTAGACTCTTTGTGACGTCAATCATG
CAGGAATTTGAAAGTGACACGTTTTTCCCAGAAATTGATTTGGGGAAATATAAACTTCTCCCAGAATACC
CAGGCGTGACGTCAGAGGTCCAGGAGGAAAAAGGCATCAAGTATAAGTTTGAAGTCTACGAGAAGAAAGA
CTAAGCTTAA
Amino acid secuence of an exemplary murine dihydrofolate reductase engineered to include
CRE binding sites (SEQ ID NO: 37)
MVRPLNCIVAVSQNMGIGKNGDLPWPPLRNEFKYFQRMTSTSSVEGKQNLVIMGRKTWFSIPEKNRPLKDRINIVTS
RELKEPPRGAHFLAKSLDDALRLIEQPELTSKVDMVWIVGGSSVYQEAMNQPGHLRLFVTSIMQEFESDTFFPEIDL
GKYKLLPEYPGVTSEVQEEKGIKYKFEVYEKKD
Nucleotide seguence of an exemplary murine dihydrofolate reductase gene engineered to
include CCAATbinding sites (SEQ ID NO: 38)
ATGGTTCGACCATTGAACTGCATCGTCGCCGTGTCCCAAAATATGGGGATTGGCAAGAACGGAGACCTACCCTGGCC
TCCATTGCGCAATGAGTTCAAGTACTTCCAAAGAATGACCACAACCTCTTCAGTGGAAGGTAAACAGAATCTGGTGA
TTATGGGTAGGAAAACCTGGTTCTCCATTCCTGAGAAGAATCGACCATTGCGCAATAGAATTAATATAGTTCTCAGT
AGAGAATTGCGCAATCCACCACGAGGAGCTCATTTTATTGCGCAATCCTTGGATGATGCATTGCGCAATATTGAACA
ACCGGAATTGGCGAGCAAAGTAGACATGGTTTGGATCGTCGGAGGCAGTTCTGTTTACCAGGAAGCCATGAATCAAC
CAGGCCACCTTAGACTCTTTGTGACAAGGATCATGCAGGAATTTGAAAGTGACACGTTTTTCCCAGAAATTGATTTG
GGGAAATATAAACTTCTCCCAGAATACCCAGGCGTCCTCTCTGAATTGCGCAATGAAAAAGGCATCAAGTATAAGTT
TGAAGTCTACGAGAAGAAAGAC TAAGCTTAA
Amino acid secuence of an exemplary murine dihydrofolate reductase engineered to include
CCAAT binding sites (SEQ ID NO: 39)
MVRPLNCIVAVSQNMGIGKNGDLPWPPLRNEFKYFQRMTTTSSVEGKQNLVIMGRKTWFSIPEKNRPLRNRINIVLS
RELRNPPRGAHFIAQSLDDALRNIEQPELASKVDMVWIVGGSSVYQEAMNQPGHLRLFVTRIMQEFESDTFFPEIDL
GKYKLLPEYPGVLSEVQEEKGIKYKFEVYEKKD
Nucleotide seguence of an exemplary murine dihydrofolate reductase gene engineered to
include Eboxes (SEQ ID NO: 40)
ATGGTTCGACCATTGAACTGCATCGTCGCCGTGTCCCAAAATATGGGGATTGGCAAGAACGGAGACCTAC
CCTGGCCTCCGCTCAGGAACGAGTTCAAGTACTTCCAAAGAATGACCACAACCTCTTCAGTGGAAGGTAA
ACAGAATCTGGTGATTATGGGTAGGCGCACGTGGTTCTCCATTCCTGAGAAGAATCGACCTTTAAAGGAC
AGAATTAATATAGTTCTCTCACGTGAACTCAAAGAACCACCACGTGGAGCTCACGTGCTTGCCAAATCAC
TGGATGATGCATTAAGACTTATTGAACAACCGGAATTGGCGTCACGTGTAGACATGGTTTGGATCGTCGG
AGGCAGTTCTGTTTACCAGGAAGCCATGAATCAACCAGGCCACGTGAGACTCTTTGTGACACGTGTCATG
CAGGAATTTGAAAGTGACACGTTTTTCCCAGAAATTGATTTGGGGAAATATAAACTTCTCCCAGAATACC
CAGGCGTCCTCTCACGTGTCCAGGAGGAAAAAGGCATCAAGTATAAGTTTGAAGTCTACGAGAAGAAAGA
CTAAGCTTAA
Amino acid seauence of an exemolarv murine dihvdrofolate reductase enaineered to include
Eboxes (SEQ ID NO: 41)
MVRPLNCIVAVSQNMGIGKNGDLPWPPLRNEFKYFQRMTTTSSVEGKQNLVIMGRRTWFSIPEKNRPLKDRINIVLS
RELKEPPRGAHVLAKSLDDALRLIEQPELASRVDMVWIVGGSSVYQEAMNQPGHVRLFVTRVMQEFESDTFFPEIDL
GKYKLLPEYPGVLSRVQEEKGIKYKFEVYEKKD
Nucleotide seauence of p300-mDHFR olasmid used in Examoles (SEQ ID NO: 42)
CTCGAGAAATCATAAAAAATTTATTTGCTTTGTGAGCGGATAACAATTATAATAGATTCAATTGTGAGCGGATAACA
ATTTCACACAGAATTCATTAAAGAGGAGAAATTAAGCATGCACCATCACCATCACCATgctagcgttcgaccattga
actgcatcgtcgccgtgagtcagaatatggggattggcaagaacggagacctaccctggcctccgctcaggaatgag
tcaaagtacttccaaagaatgactcagactgactcagttgagtcaaaacagaatctggtgattatgggtaggaaaac
ctggttctccattcctgagtcaaatcgacctttaaaggacagaattaatatagttctgagtcaagaactcaaagaac
caccacgaggagctcattttcttgccaaaagtttggatgatgccttaagacttattgagtcaccggaattggcgagc
aaagttgactcagtttggatcgtcggaggcagttctgtttaccaggaagccatgactcaaccaggccaccttagact
ctttgtgactcagatcatgcaggaatttgagtcagacacgtttttcccagaaattgactcagggaaatataaacttc
tccctgagtcaccaggcgtcctgagtcaggtccaggaggaaaaaggcatcaagtataagtttgaagtctacgagaag
aaagactaagcttAATTAGCTGAGCTTGGACTCCTGTTGATAGATCCAGTAATGACCTCAGAACTCCATCTGGATTT
GTTCAGAACGCTCGGTTGCCGCCGGGCGTTTTTTATTGGTGAGAATCCAAGCTAGTTTGGGAGGTTCCAACTTTCAC
CATAATGAAATAAGATCACTACCGGGCGTATTTTTTGAGTTATCGAGATTTTCAGGAGCTAAGGAAGCTAAAATGGA
GAAAAAAATCACTGGATATACCACCGTTGATATATCCCAATGGCATCGTAAAGAACATTTTGAGGCATTTCAGTCAG
TTGCTCAATGTACCTATAACCAGACCGTTCAGCTGGATATTACGGCCTTTTTAAAGACCGTAAAGAAAAATAAGCAC
AAGTTTTATCCGGCCTTTATTCACATTCTTGCCCGCCTGATGAATGCTCATCCGGAGTTCCGTATGGCAATGAAAGA
CGGTGAGCTGGTGATATGGGATAGTGTTCACCCTTGTTACACCGTTTTCCATGAGCAAACTGAAACGTTTTCATCGC
TCTGGAGTGAATACCACGACGATTTCCGGCAGTTTCTACACATATATTCGCAAGATGTGGCGTGTTACGGTGAAAAC
CTGGCCTATTTCCCTAAAGGGTTTATTGAGAATATGTTTTTCGTCTCAGCCAATCCCTGGGTGAGTTTCACCAGTTT
TGATTTAAACGTGGCCAATATGGACAACTTCTTCGCCCCCGTTTTCACCATGGGCAAATATTATACGCAAGGCGACA
AGGTGCTGATGCCGCTGGCGATTCAGGTTCATCATGCCGTTTGTGATGGCTTCCATGTCGGCAGAATGCTTAATGAA
TTACAACAGTACTGCGATGAGTGGCAGGGCGGGGCGTAATTTTTTTAAGGCAGTTATTGGTGCCCTTAAACGCCTGG
GGTAATGACTCTCTAGCTTGAGGCATCAAATAAAACGAAAGGCTCAGTCGAAAGACTGGGCCTTTCGTTTTATCTGT
TGTTTGTCGGTGAACGCTCTCCTGAGTAGGACAAATCCGCCCTCTAGAGCTGCCTCGCGCGTTTCGGTGATGACGGT
GAAAACCTCTGACACATGCAGCTCCCGGAGACGGTCACAGCTTGTCTGTAAGCGGATGCCGGGAGCAGACAAGCCCG
TCAGGGCGCGTCAGCGGGTGTTGGCGGGTGTCGGGGCGCAGCCATGACCCAGTCACGTAGCGATAGCGGAGTGTATA
CTGGCTTAACTATGCGGCATCAGAGCAGATTGTACTGAGAGTGCACCATATGCGGTGTGAAATACCGCACAGATGCG
TAAGGAGAAAATACCGCATCAGGCGCTCTTCCGCTTCCTCGCTCACTGACTCGCTGCGCTCGGTCGTTCGGCTGCGG
CGAGCGGTATCAGCTCACTCAAAGGCGGTAATACGGTTATCCACAGAATCAGGGGATAACGCAGGAAAGAACATGTG
AGCAAAAGGCCAGCAAAAGGCCAGGAACCGTAAAAAGGCCGCGTTGCTGGCGTTTTTCCATAGGCTCCGCCCCCCTG
ACGAGCATCACAAAAATCGACGCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGTCCGCCTTTCTCCCTTCGGG
AAGCGTGGCGCTTTCTCATAGCTCACGCTGTAGGTATCTCAGTTCGGTGTAGGTCGTTCGCTCCAAGCTGGGCTGTG
TGCACGAACCCCCCGTTCAGCCCGACCGCTGCGCCTTATCCGGTAACTATCGTCTTGAGTCCAACCCGGTAAGACAC
GACTTATCGCCACTGGCAGCAGCCACTGGTAACAGGATTAGCAGAGCGAGGTATGTAGGCGGTGCTACAGAGTTCTT
GAAGTGGTGGCCTAACTACGGCTACACTAGAAGGACAGTATTTGGTATCTGCGCTCTGCTGAAGCCAGTTACCTTCG
GAAAAAGAGTTGGTAGCTCTTGATCCGGCAAACAAACCACCGCTGGTAGCGGTGGTTTTTTTGTTTGCAAGCAGCAG
ATTACGCGCAGAAAAAAAGGATCTCAAGAAGATCCTTTGATCTTTTCTACGGGGTCTGACGCTCAGTGGAACGAAAA
CTCACGTTAAGGGATTTTGGTCATGAGATTATCAAAAAGGATCTTCACCTAGATCCTTTTAAATTAAAAATGAAGTT
TTAAATCAATCTAAAGTATATATGAGTAAACTTGGTCTGACAGTTACCAATGCTTAATCAGTGAGGCACCTATCTCA
GCGATCTGTCTATTTCGTTCATCCATAGTTGCCTGACTCCCCGTCGTGTAGATAACTACGATACGGGAGGGCTTACC
ATCTGGCCCCAGTGCTGCAATGATACCGCGAGACCCACGCTCACCGGCTCCAGATTTATCAGCAATAAACCAGCCAG
CCGCGTTCTTCGGGGCGAAAACTCTCAAGGATCTTACCGCTGTTGAGATCCAGTTCGATGTAACCCACTCGTGCACC
CAACTGATCTTCAGCATCTTTTACTTTCACCAGCGTTTCTGGGTGAGCAAAAACAGGAAGGCAAAATGCCGCAAAAA
AGGGAATAAGGGCGACACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCCGCGCACATTTCCCCG
AAAAGTGCCACCTGACGTCTAAGAAACCATTATTATCATGACATTAACCTATAAAAATAGGCGTATCACGAGGCCCT
TTCGTCTTCAC
Nucleotide sequence of p230d-basic-cJun plasmid used in Examples (SEQ ID NO: 43)
CTCGAGAAATCATAAAAAATTTATTTGCTTTGTGAGCGGATAACAATTATAATAGATTCAATTGTGAGCGGATAACA
ATTTCACACAGAATTCATTAAAGAGGAGAAATTAAGCATGCGCATTAAAGCCGAACGCAAACGGATGCGCAACCGCA
TCGCAGCCTCCAAGTGCCGCAAACGCAAATTGGAGCGCATCGCCCGCTTGGAAGAAAAGGTGAAAACCCTGAAAGCA
CAGAACTATGAGCTGGCCTCCACCGCCAACATGTTGCGCGAACAGGTGGCCCAGCTCGGCGCGCCTCATCACCATCA
CCATCACTGATAAAGCGCGCCTTGATAAGCTTAATTAGCTGAGCTTGGACTCCTGTTGATAGATCCAGTAATGACCT
CAGAACTCCATCTGGATTTGTTCAGAACGCTCGGTTGCCGCCGGGCGTTTTTTATTGGTGAGAATCCAGGCGAGATT
TTCAGGAGCTAAGGAAGCTAAAATGGAGAAAAAAATCACTGGATATACCACCGTTGATATATCCCAATGGCATCGTA
AAGAACATTTTGAGGCATTTCAGTCAGTTGCTCAATGTACCTATAACCAGACCGTTCAGCTGGATATTACGGCCTTT
TTAAAGACCGTAAAGAAAAATAAGCACAAGTTTTATCCGGCCTTTATTCACATTCTTGCCCGCCTGATGAATGCTCA
TCCGGAATTTCGTATGGCAATGAAAGACGGTGAGCTGGTGATATGGGATAGTGTTCACCCTTGTTACACCGTTTTCC
ATGAGCAAACTGAAACGTTTTCATCGCTCTGGAGTGAATACCACGACGATTTCCGGCAGTTTCTACACATATATTCG
CAAGATGTGGCGTGTTACGGTGAAAACCTGGCCTATTTCCCTAAAGGGTTTATTGAGAATATGTTTTTCGTCTCAGC
CAATCCCTGGGTGAGTTTCACCAGTTTTGATTTAAACGTGGCCAATATGGACAACTTCTTCGCCCCCGTTTTCACCA
TGGGCAAATATTATACGCAAGGCGACAAGGTGCTGATGCCGCTGGCGATTCAGGTTCATCATGCCGTTTGTGATGGC
TTCCATGTCGGCAGAATGCTTAATGAATTACAACAGTACTGCGATGAGTGGCAGGGCGGGGCGTAATTTTTTTAAGG
CAGTTATTGGTGCCCTTAAACGCCTGGGGTAATGACTCTCTAGCTTGAGGCATCAAATAAAACGAAAGGCTCAGTCG
AAAGACTGGGCCTTTCGTTTTATCTGTTGTTTGTCGGTGAACGCTCTCCTGAGTAGGACAAATCCGCCCTCTAGAGC
TGCCTCGCGCGTTTCGGTGATGACGGTGAAAACCTCTGACACATGCAGCTCCCGGAGACGGTCACAGCTTGTCTGTA
AGCGGATGCCGGGAGCAGACAAGCCCGTCAGGGCGCGTCAGCGGGTGTTGGCGGGTGTCGGGGCGCAGCCATGACCC
AGTCACGTAGCGATAGCGGAGTGTATACTGGCTTAACTATGCGGCATCAGAGCAGATTGTACTGAGAGTGCACCATA
TGCGGTGTGAAATACCGCACAGATGCGTAAGGAGAAAATACCGCATCAGGCGCTCTTCCGCTTCCTCGCTCACTGAC
TCGCTGCGCTCGGTCGTTCGGCTGCGGCGAGCGGTATCAGCTCACTCAAAGGCGGTAATACGGTTATCCACAGAATC
AGGGGATAACGCAGGAAAGAACATGTGAGCAAAAGGCCAGCAAAAGGCCAGGAACCGTAAAAAGGCCGCGTTGCTGG
CGTTTTTCCATAGGCTCCGCCCCCCTGACGAGCATCACAAAAATCGACGCTCAAGTCAGAGGTGGCGAAACCCGACA
GGACTATAAAGATACCAGGCGTTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCTGTAGGTATCTCAGTTCGGTGT
AGGTCGTTCGCTCCAAGCTGGGCTGTGTGCACGAACCCCCCGTTCAGCCCGACCGCTGCGCCTTATCCGGTAACTAT
CGTCTTGAGTCCAACCCGGTAAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAGAAGGACAGTATTTGGTATCT
GCGCTCTGCTGAAGCCAGTTACCTTCGGAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATCTCAAGAAGATCCTTTGATCTTTTCTAC
GGGGTCTGACGCTCAGTGGAACGAAAACTCACGTTAAGGGATTTTGGTCATGAGATTATCAAAAAGGATCTTCACCT
AGATCCTTTTAAATTAAAAATGAAGTTTTAAATCAATCTAAAGTATATATGAGTAAACTTGGTCTGACAGTTACCAA
TGCTTAATCAGTGAGGCACCTATCTCAGCGATCTGTCTATTTCGTTCATCCATAGTTGCCTGACTCCCCGTCGTGTA
GATAACTACGATACGGGAGGGCTTACCATCTGGCCCCAGTGCTGCAATGATACCGCGAGACCCACGCTCACCGGCTC
CAGATTTATCAGCAATAAACCAGCCAGCCGGAAGGGCCGAGCGCAGAAGTGGTCCTGCAACTTTATCCGCCTCCATC
CAGTCTATTAATTGTTGCCGGGAAGCTAGAGTAAGTAGTTCGCCAGTTAATAGTTTGCGCAACGTTGTTGCCATTGC
TACAGGCATCGTGGTGTCACGCTCGTCGTTTGGTATGGCTTCATTCAGCTCCGGTTCCCAACGATCAAGGCGAGTTA
CATGATCCCCCATGTTGTGCAAAAAAGCGGTTAGCTCCTTCGGTCCTCCGATCGTTGTCAGAAGTAAGTTGGCCGCA
GTGTTATCACTCATGGTTATGGCAGCACTGCATAATTCTCTTACTGTCATGCCATCCGTAAGATGCTTTTCTGTGAC
TGGTGAGTACTCAACCAAGTCATTCTGAGAATAGTGTATGCGGCGACCGAGTTGCTCTTGCCCGGCGTCAATACGGG
ATAATACCGCGCCACATAGCAGAACTTTAAAAGTGCTCATCATTGGAAAACGTTCTTCGGGGCGAAAACTCTCAAGG
ATCTTACCGCTGTTGAGATCCAGTTCGATGTAACCCACTCGTGCACCCAACTGATCTTCAGCATCTTTTACTTTCAC
CAGCGTTTCTGGGTGAGCAAAAACAGGAAGGCAAAATGCCGCAAAAAAGGGAATAAGGGCGACACGGAAATGTTGAA
TACTCATACTCTTCCTTTTTCAATATTATTGAAGCATTTATCAGGGTTATTGTCTCATGAGCGGATACATATTTGAA
TGTATTTAGAAAAATAAACAAATAGGGGTTCCGCGCACATTTCCCCGAAAAGTGCCACCTGACGTCTAAGAAACCAT
TATTATCATGACATTAACCTATAAAAATAGGCGTATCACGAGGCCCTTTCGTCTTCAC
Nucleotide seauence ofoREP4 exoressina the lac repressor used in Examples (SEQ ID NO:
44)
AAGCTTCACGCTGCCGCAAGCACTCAGGGCGCAAGGGCTGCTAAAGGAAGCGGAACACGTAGAAAGCCAGTCCGCAG
AAACGGTGCTGACCCCGGATGAATGTCAGCTACTGGGCTATCTGGACAAGGGAAAACGCAAGCGCAAAGAGAAAGCA
GGTAGCTTGCAGTGGGCTTACATGGCGATAGCTAGACTGGGCGGTTTTATGGACAGCAAGCGAACCGGAATTGCCAG
CTGGGGCGCCCTCTGGTAAGGTTGGGAAGCCCTGCAAAGTAAACTGGATGGCTTTCTTGCCGCCAAGGATCTGATGG
CGCAGGGGATCAAGATCTGATCAAGAGACAGGATGACGGTCGTTTCGCATGCTTGAACAAGATGGATTGCACGCAGG
TTCTCCGGCCGCTTGGGTGGAGAGGCTATTCGGCTATGACTGGGCACAACAGACAATCGGCTGCTCTGATGCCGCCG
TGTTCCGGCTGTCAGCGCAGGGGCGCCCGGTTCTTTTTGTCAAGACCGACCTGTCCGGTGCCCTGAATGAACTGCAG
GACGAGGCAGCGCGGCTATCGTGGCTGGCCACGACGGGCGTTCCTTGCGCAGCTGTGCTCGACGTTGTCACTGAAGC
GGGAAGGGACTGGCTGCTATTGGGCGAAGTGCCGGGGCAGGATCTCCTGTCATCTCACCTTGCTCCTGCCGAGAAAG
TATCCATCATGGCTGATGCAATGCGGCGGCTGCATACGCTTGATCCGGCTACCTGCCCATTCGACCACCAAGCGAAA
CATCGCATCGAGCGAGCACGTACTCGGATGGAAGCCGGTCTTGTCGATCAGGATGATCTGGACGAAGAGCATCAGGG
GCTCGCGCCAGCCGAACTGTTCGCCAGGCTCAAGGCGCGCATGCCCGACGGCGAGGATCTCGTCGTGACCCATGGCG
ATGCCTGCTTGCCGAATATCATGGTGGAAAATGGCCGCTTTTCTGGATTCATCGACTGTGGCCGGCTGGGTGTGGCG
GACCGCTATCAGGACATAGCGTTGGCTACCCGTGATATTGCTGAAGAGCTTGGCGGCGAATGGGCTGACCGCTTCCT
CGTGCTTTACGGTATCGCCGCTCCCGATTCGCAGCGCATCGCCTTCTATCGCCTTCTTGACGAGTTCTTCTGAGCGG
GACTCTGGGGTTCGAAATGACCGACCAAGCGACGCCCAACCTGCCATCACGAGATTTCGATTCCACCGCCGCCTTCT
ATGAAAGGTTGGGCTTCGGAATCGTTTTCCGGGACGCCGGCTGGATGATCCTCCAGCGCGGGGATCTCATGCTGGAG
TTCTTCGCCCACCCCGGGCTCGATCCCCTCGCGAGTTGGTTCAGCTGCTGCCTGAGGCTGGACGACCTCGCGGAGTT
CTACCGGCAGTGCAAATCCGTCGGCATCCAGGAAACCAGCAGCGGCTATCCGCGCATCCATGCCCCCGAACTGCAGG
AGTGGGGAGGCACGATGGCCGCTTTGGTCGACAATTCGCGCTAACTTACATTAATTGCGTTGCGCTCACTGCCCGCT
TTCCAGTCGGGAAACCTGTCGTGCCAGCTGCATTAATGAATCGGCCAACGCGCGGGGAGAGGCGGTTTGCGTATTGG
GCGCCAGGGTGGTTTTTCTTTTCACCAGTGAGACGGGCAACAGCTGATTGCCCTTCACCGCCTGGCCCTGAGAGAGT
TGCAGCAAGCGGTCCACGCTGGTTTGCCCCAGCAGGCGAAAATCCTGTTTGATGGTGGTTAACGGCGGGATATAACA
TGAGCTGTCTTCGGTATCGTCGTATCCCACTACCGAGATATCCGCACCAACGCGCAGCCCGGACTCGGTAATGGCGC
GCATTGCGCCCAGCGCCATCTGATCGTTGGCAACCAGCATCGCAGTGGGAACGATGCCCTCATTCAGCATTTGCATG
GTTTGTTGAAAACCGGACATGGCACTCCAGTCGCCTTCCCGTTCCGCTATCGGCTGAATTTGATTGCGAGTGAGATA
TTTATGCCAGCCAGCCAGACGCAGACGCGCCGAGACAGAACTTAATGGGCCCGCTAACAGCGCGATTTGCTGGTGAC
CCAATGCGACCAGATGCTCCACGCCCAGTCGCGTACCGTCTTCATGGGAGAAAATAATACTGTTGATGGGTGTCTGG
TCAGAGACATCAAGAAATAACGCCGGAACATTAGTGCAGGCAGCTTCCACAGCAATGGCATCCTGGTCATCCAGCGG
ATAGTTAATGATCAGCCCACTGACGCGTTGCGCGAGAAGATTGTGCACCGCCGCTTTACAGGCTTCGACGCCGCTTC
GTTCTACCATCGACACCACCACGCTGGCACCCAGTTGATCGGCGCGAGATTTAATCGCCGCGACAATTTGCGACGGC
GCGTGCAGGGCCAGACTGGAGGTGGCAACGCCAATCAGCAACGACTGTTTGCCCGCCAGTTGTTGTGCCACGCGGTT
GGGAATGTAATTCAGCTCCGCCATCGCCGCTTCCACTTTTTCCCGCGTTTTCGCAGAAACGTGGCTGGCCTGGTTCA
CCACGCGGGAAACGGTCTGATAAGAGACACCGGCATACTCTGCGACATCGTATAACGTTACTGGTTTCACATTCACC
ACCCTGAATTGACTCTCTTCCGGGCGCTATCATGCCATACCGCGAAAGGTTTTGCGCCATTCGATGGTGTCAACGTA
AATGCATGCCGCTTCGCCTTCGCGCGCGAATTGTCGACCCTGTCCCTCCTGTTCAGCTACTGACGGGGTGGTGCGTA
ACGGCAAAAGCACCGCCGGACATCAGCGCTAGCGGAGTGTATACTGGCTTACTATGTTGGCACTGATGAGGGTGTCA
GTGAAGTGCTTCATGTGGCAGGAGAAAAAAGGCTGCACCGGTGCGTCAGCAGAATATGTGATACAGGATATATTCCG
CTTCCTCGCTCACTGACTCGCTACGCTCGGTCGTTCGACTGCGGCGAGCGGAAATGGCTTACGAACGGGGCGGAGAT
TTCCTGGAAGATGCCAGGAAGATACTTAACAGGGAAGTGAGAGGGCCGCGGCAAAGCCGTTTTTCCATAGGCTCCGC
CCCCCTGACAAGCATCACGAAATCTGACGCTCAAATCAGTGGTGGCGAAACCCGACAGGACTATAAAGATACCAGGC
GTTTCCCCTGGCGGCTCCCTCGTGCGCTCTCCTGTTCCTGCCTTTCGGTTTACCGGTGTCATTCCGCTGTTATGGCC
GCGTTTGTCTCATTCCACGCCTGACACTCAGTTCCGGGTAGGCAGTTCGCTCCAAGCTGGACTGTATGCACGAACCC
CCCGTTCAGTCCGACCGCTGCGCCTTATCCGGTAACTATCGTCTTGAGTCCAACCCGGAAAGACATGCAAAAGCACC
ACTGGCAGCAGCCACTGGTAATTGATTTAGAGGAGTTAGTCTTGAAGTCATGCGCCGGTTAAGGCTAAACTGAAAGG
ACAAGTTTTGGTGACTGCGCTCCTCCAAGCCAGTTACCTCGGTTCAAAGAGTTGGTAGCTCAGAGAACCTTCGAAAA
ACCGCCCTGCAAGGCGGTTTTTTCGTTTTCAGAGCAAGAGATTACGCGCAGACCAAAACGATCTCAAGAAGATCATC
TTATTAATCAGATAAAATATTTCTAGATTTCAGTGCAATTTATCTCTTCAAATGTAGCACCTGAAGTCAGCCCCATA
CGATATAAGTTGTTAATTCTCATGTTTGACAGCTTATCATCGAT
Nucleotide sequence of control cJun plasmid lacking the DNA-binding basic region used in
Examples (SEQ ID NO: 45)
CTCGAGAAATCATAAAAAATTTATTTGCTTTGTGAGCGGATAACAATTATAATAGATTCAATTGTGAGCGGATAACA
ATTTCACACAGAATTCATTAAAGAGGAGAAATTAAGCATGCACCATCACCATCACCATGCTAGCATCGCCCGGCTGG
AGGAAAAAGTGAAGACCTTGAAGGCCCAGAACTATGAGCTGGCGTCCACGGCCAACATGCTCCGGGAACAGGTGGCA
CAGCTTGGCGCGCCTTAAGGTAGCTCTAAGCTTAATTAGCTGAGCTTGGACTCCTGTTGATAGATCCAGTAATGACC
TCAGAACTCCATCTGGATTTGTTCAGAACGCTCGGTTGCCGCCGGGCGTTTTTTATTGGTGAGAATCCAGGCGAGAT
TTTCAGGAGCTAAGGAAGCTAAAATGGAGAAAAAAATCACTGGATATACCACCGTTGATATATCCCAATGGCATCGT
AAAGAACATTTTGAGGCATTTCAGTCAGTTGCTCAATGTACCTATAACCAGACCGTTCAGCTGGATATTACGGCCTT
TTTAAAGACCGTAAAGAAAAATAAGCACAAGTTTTATCCGGCCTTTATTCACATTCTTGCCCGCCTGATGAATGCTT
AATGAATTACAACAGTACTGCGATGAGTGGCAGGGCGGGGCGTAATTTTTTTAAGGCAGTTATTGGTGCCCTTAAAC
GCCTGGGGTAATGACTCTCTAGCTTGAGGCATCAAATAAAACGAAAGGCTCAGTCGAAAGACTGGGCCTTTCGTTTT
ATCTGTTGTTTGTCGGTGAACGCTCTCCTGAGTAGGACAAATCCGCCCTCTAGAGCTGCCTCGCGCGTTTCGGTGAT
GACGGTGAAAACCTCTGACACATGCAGCTCCCGGAGACGGTCACAGCTTGTCTGTAAGCGGATGCCGGGAGCAGACA
AGCCCGTCAGGGCGCGTCAGCGGGTGTTGGCGGGTGTCGGGGCGCAGCCATGACCCAGTCACGTAGCGATAGCGGAG
TGTATACTGGCTTAACTATGCGGCATCAGAGCAGATTGTACTGAGAGTGCACCATATGCGGTGTGAAATACCGCACA
GATGCGTAAGGAGAAAATACCGCATCAGGCGCTCTTCCGCTTCCTCGCTCACTGACTCGCTGCGCTCGGTCGTTCGG
CTGCGGCGAGCGGTATCAGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGTTCGGTGTAGGTCGTTCGCTCCAAGCTGG
GCTGTGTGCACGAACCCCCCGTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAGGTATGTAGGCGGTGCTACAGA
GTTCTTGAAGTGGTGGCCTAACTACGGCTACACTAGAAGGACAGTATTTGGTATCTGCGCTCTGCTGAAGCCAGTTA
CCTTCGGAAAAAGAGTTGGTAGCTCTTGATCCGGCAAACAAACCACCGCTGGTAGCGGTGGTTTTTTTGTTTGCAAG
CAGCAGATTACGCGCAGAAAAAAAGGATCTCAAGAAGATCCTTTGATCTTTTCTACGGGGTCTGACGCTCAGTGGAA
CGAAAACTCACGTTAAGGGATTTTGGTCATGAGATTATCAAAAAGGATCTTCACCTAGATCCTTTTAAATTAAAAAT
GAAGTTTTAAATCAATCTAAAGTATATATGAGTAAACTTGGTCTGACAGTTACCAATGCTTAATCAGTGAGGCACCT
ATCTCAGCGATCTGTCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTTATCAGCAATAAACC
AGCCAGCCGGAAGGGCCGAGCGCAGAAGTGGTCCTGCAACTTTATCCGCCTCCATCCAGTCTATTAATTGTTGCCGG
GAAGCTAGAGTAAGTAGTTCGCCAGTTAATAGTTTGCGCAACGTTGTTGCCATTGCTACAGGCATCGTGGTGTCACG
CTCGTCGTTTGGTATGGCTTCATTCAGCTCCGGTTCCCAACGATCAAGGCGAGTTACATGATCCCCCATGTTGTGCA
AAAAAGCGGTTAGCTCCTTCGGTCCTCCGATCGTTGTCAGAAGTAAGTTGGCCGCAGTGTTATCACTCATGGTTATG
GCAGCACTGCATAATTCTCTTACTGTCATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAATACGGGATAATACCGCGCCACATAGCA
GAACTTTAAAAGTGCTCATCATTGGAAAACGTTCTTCGGGGCGAAAACTCTCAAGGATCTTACCGCTGTTGAGATCC
AGTTCGATGTAACCCACTCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACTCATACTCTTCCTTTTTC
AATATTATTGAAGCATTTATCAGGGTTATTGTCTCATGAGCGGATACATATTTGAATGTATTTAGAAAAATAAACAA
ATAGGGGTTCCGCGCACATTTCCCCGAAAAGTGCCACCTGACGTCTAAGAAACCATTATTATCATGACATTAACCTA
TAAAAATAGGCGTATCACGAGGCCCTTTCGTCTTCAC
Nucleotide sequence of basic-c-Jun (SEQ ID NO: 46)
CGCATTAAAGCCGAACGCAAACGGATGCGCAACCGCATCGCAGCCTCCAAGTGCCGCAAACGCAAATTGGAGCGC
Amino acid sequence of basic-c-Jun (SEQ ID NO: 47)
RIKAERKRMRNRIAASKCRKRKLER
Nucleotide sequence of Aβ₁₋₄₂ (SEQ ID NO: 48)
GACGCTGAATTTCGCCACGACTCCGGCTATGAGGTACACCACCAGAAACTGGTTTTTTTTGCTGAGGACGTTGGCTC
CAACAAAGGTGCTATCATCGGTCTGATGGTTGGCGGCGTTGTTATCGCTTAA
Amino acid sequence of Aβ₁₋₄₂ (SEQ ID NO: 49)
DAEFRHDSGYEVHHQKLVFFAEDVGSNKGAIIGLMVGGVVIA
Nucleotide sequence of basic-Aβ₁₋₄₂ (SEQ ID NO: 50)
Sequence encoding Aβ₁₋₄₂ underlined
ATGCGCATTAAAGCCGAACGCAAACGGATGCGCAACCGCATCGCAGCCTCCAAGTGCCGCAAACGCAAATTGGAGCG
CGACGCTGAATTTCGCCACGACTCCGGCTATGAGGTACACCACCAGAAACTGGTTTTTTTTGCTGAGGACGTTGGCT
CCAACAAAGGTGCTATCATCGGTCTGATGGTTGGCGGCGTTGTTATCGCTTAA
Amino acid sequence of basic-Aβ₁₋₄₂ (SEQ ID NO: 51)
Sequence encoding Aβ₁₋₄₂ underlined
MRIKAERKRMRNRIAASKCRKRKLERDAEFRHDSGYEVHHQKLVFFAEDVGSNKGAIIGLMVGGVVIA
Nucleotide sequence of αS (SEQ ID NO: 52)
GATGTATTCATGAAAGGACTTTCAAAGGCCAAGGAGGGAGTTGTGGCTGCTGCTGAGAAAACCAAACAGGGTGTGGC
AGAAGCAGCAGGAAAGACAAAAGAGGGTGTTCTCTATGTAGGCTCCAAAACCAAGGAGGGAGTGGTGCATGGTGTGG
CAACAGTGGCTGAGAAGACCAAAGAGCAAGTGACAAATGTTGGAGGAGCAGTGGTGACGGGTGTGACAGCAGTAGCC
CAGAAGACAGTGGAGGGAGCAGGGAGCATTGCAGCAGCCACTGGCTTTGTCAAAAAGGACCAGTTGGGCAAGAATGA
AGAAGGAGCCCCACAGGAAGGAATTCTGGAAGATATGCCTGTGGATCCTGACAATGAGGCTTATGAAATGCCTTCTG
AGGAAGGGTATCAAGACTACGAACCTGAAGCCTAA
Amino acid sequence of αS (SEQ ID NO: 53)
DVFMKGLSKAKEGVVAAAEKTKQGVAEAAGKTKEGVLYVGSKTKEGVVHGVATVAEKTKEQVTNVGGAVVTGVTAVA
QKTVEGAGSIAAATGFVKKDQLGKNEEGAPQEGILEDMPVDPDNEAYEMPSEEGYQDYEPEA
Nucleotide sequence of basic-αS (SEQ ID NO: 54)
Sequence encoding αS underlined
ATGCGCATTAAAGCCGAACGCAAACGGATGCGCAACCGCATCGCAGCCTCCAAGTGCCGCAAACGCAAATTGGAGCG
CGATGTGTTTATGAAAGGTCTGAGCAAAGCGAAAGAAGGCGTGGTGGCTGCGGCGGAAAAAACGAAACAGGGCGTGG
CGGAAGCGGCCGGCAAAACGAAAGAAGGTGTTCTGTATGTCGGCAGCAAAACCAAAGAAGGCGTGGTTCATGGTGTG
GCCACCGTTGCAGAAAAAACGAAAGAACAGGTCACCAACGTGGGCGGTGCTGTCGTGACCGGTGTTACGGCTGTCGC
GCAAAAAACGGTGGAAGGCGCGGGTTCTATTGCGGCGGCAACCGGTTTCGTTAAAAAAGATCAGCTGGGTAAAAATG
AAGAAGGCGCGCCGCAAGAAGGTATCCTGGAAGACATGCCGGTGGATCCGGACAACGAAGCGTATGAAATGCCGTCG
GAAGAAGGCTATCAAGACTATGAACCGGAAGCGTAATGA
Amino acid sequence of basic-αS (SEQ ID NO: 55)
Sequence encoding αS underlined
MRIKAERKRMRNRIAASKCRKRKLERDVFMKGLSKAKEGVVAAAEKTKQGVAEAAGKTKEGVLYVGSKTK
EGVVHGVATVAEKTKEQVTNVGGAVVTGVTAVAQKTVEGAGSIAAATGFVKKDQLGKNEEGAPQEGILED
MPVDPDNEAYEMPSEEGYQDYEPEA
Nucleic acid sequences of binding sites
CAAT box GGCCAATCT (SEQ ID NO: 35)
CArG box CC(A/T₆)GG (SEQ ID NO: 56)
E2 box CAGGTG and CACCTG (SEQ ID NOs: 57 and 58)
HY box TG(A/T)GGG (SEQ ID NO: 59)
T box TCACACCT (SEQ ID NO: 60)
TATA box TATAAA (SEQ ID NO: 61)
X box GTTGGCATGGCAAC (SEQ ID NO: 62)
Y box (A/G)CTAACC(A/G)(A/G)(C/T) (SEQ ID NO: 63)
ATA box AAATAT (SEQ ID NO: 64)
CGCG box (A/C/G)CGCG(C/G/T) (SEQ ID NO: 65)
DREB box TACCGACAT (SEQ ID NO: 66)
Fur box GATAATGATAATCATTATC (SEQ ID NO: 67)
G box GCCACGTGGC (SEQ ID NO: 68)
GCC box AGCCGCC (SEQ ID NO: 69)
H box ACACCA (SEQ ID NO: 70)
Prolamin box TGTAAAG (SEQ ID NO: 71)
Pyrimidine box CCTTTT (SEQ ID NO: 72)
TACTAAC box ATTTACTAAC (SEQ ID NO: 73)

Numbered Clauses

The following numbered clauses, describing aspects and embodiments of the invention, are part of the description.

1. A method for screening for an inhibitor of association between first and second candidate binding partners, the method comprising:

providing a cell, wherein the cell comprises:

a test compound;

a first hybrid protein comprising a first component of a DNA-binding protein linked to the first candidate binding partner;

a second hybrid protein comprising a second component of the DNA-binding protein linked to the second candidate binding partner; and

a reporter expression cassette that encodes a reporter expression product,

wherein the first and second hybrid proteins form a DNA-binding complex upon association of the first and second candidate binding partners, and wherein the reporter expression cassette comprises at least one binding site for the DNA-binding complex such that binding of the complex to the binding site inhibits expression of the reporter expression product; and

determining expression of the reporter expression product in the presence of the test compound;

wherein an increase in expression of the reporter expression product in the presence of the test compound indicates that the test compound is capable of inhibiting association between the first and second candidate binding partners.

2. The method of clause 1, wherein the reporter expression product is a reporter protein.

3. The method of clause 2, wherein the reporter protein is a cell survival protein, a cell reproduction protein a fluorescent protein, a bioluminescent protein, a protease, an enzyme that acts on a substrate to produce a colorimetric signal, a protein kinase, a transcriptional activator, or a regulatory protein such as ubiquitin.

4. The method of clause 3, wherein the reporter protein is a cell survival protein, optionally wherein the cell survival protein is an enzyme involved in synthesising compounds that are required for cell survival, or a protein that is able to inhibit action of a toxic agent.

5. The method of clause 3, wherein the reporter protein is a cell reproduction protein, optionally wherein the cell reproduction protein is an enzyme involved in synthesising compounds that are required for cell proliferation.

6. The method of clause 4, wherein the cell survival protein is an exogenous cell survival protein that is able to compensate for a deficiency in an endogenous cell survival protein; and

wherein the method is performed under selection conditions such that survival of the cell is dependent upon activity of the exogenous cell survival protein.

7. The method of clause 5, wherein the cell reproduction protein is an exogenous cell reproduction protein that is able to compensate for a deficiency in an endogenous cell reproduction protein; and

wherein the method is performed under selection conditions such that proliferation of the cell is dependent upon activity of the exogenous cell reproduction protein.

8. The method of clause 6 or clause 7, wherein the exogenous cell survival protein is an orthologue of the endogenous cell survival protein, or the exogenous cell reproduction protein is an orthologue of the endogenous cell reproduction protein.

9. The method of any one of clauses 6 to 8, wherein the exogenous cell survival protein or exogenous cell reproduction protein is resistant to selection conditions that inhibit the function of the endogenous cell survival protein or endogenous cell reproduction protein.

10. The method of any one of clauses 6 to 9, wherein the selection conditions comprise the addition of a selection agent that inhibits the function of the endogenous cell survival protein or endogenous cell reproduction protein.

11. The method of any one of clauses 4, 6, or 8 to 10, wherein the cell survival protein is dihydrofolate reductase (DHFR), optionally wherein the DHFR has an amino acid sequence that is at least 80% identical to the sequence set forth in SEQ ID NO: 1.

12. The method of any one of the preceding clauses, wherein the reporter expression cassette comprises between 1 and 5, between 1 and 10, between 1 and 15, between 1 and 20, between 5 and 10, between 5 and 15, between 5 and 20, between 10 and 15, between 10 and 20, between 10 and 18 or between 12 and 16 binding sites.

13. The method of any one of clauses 1 to 11, wherein the reporter expression cassette comprises at least 2, at least 5, at least 10, at least 12, or at least 15 binding sites.

14. The method of any one of clauses 2 to 13, wherein the reporter protein retains at least 50%, at least 70%, at least 90%, or at least 95% of the function of a parent reporter protein, and wherein the parent reporter protein is encoded by a parent reporter expression cassette that corresponds to the reporter expression cassette but does not comprise the binding site(s).

15. The method of any one of clauses 2 to 14, wherein some or all of the binding site(s) are located in the protein coding sequence of the reporter expression cassette.

16. The method of clauses 15, wherein the majority or all of the binding sites located in the protein coding sequence of the reporter expression cassette were introduced as silent, semi-conservative and/or conservative mutations.

17. The method of clause 15 or clause 16, wherein the majority or all of the binding sites located in the protein coding sequence of the reporter expression cassette are located at positions that encode a solvent exposed residue in the reporter protein.

18. The method of any one of clauses 15 to 17, wherein the majority or all of the binding sites located in the protein coding sequence of the reporter expression cassette are not located at positions that encode a residue that forms part of the catalytic centre of the reporter protein.

19. The method of any one of clauses 2 to 18, wherein the reporter protein has an amino acid sequence that is at least 80% identical to a parent reporter protein, wherein the parent reporter protein is encoded by a parent reporter expression cassette that corresponds to the reporter expression cassette but does not comprise the binding site(s).

20. The method of any one the preceding clauses, wherein the method comprises administering the reporter expression cassette in order to provide the cell comprising the reporter expression cassette.

21. The method of any one of the preceding clauses, wherein the first and second components of the DNA-binding protein have an identical amino acid sequence.

22. The method of any one of the preceding clauses, wherein the first and second components of the DNA-binding protein have different amino acid sequences.

23. The method of any one of the preceding clauses, wherein the first and second components of the DNA-binding protein lack a dimerization domain.

24. The method of any one of the preceding clauses, wherein the first and second components of the DNA-binding protein are DNA-binding fragments of a transcription factor.

25. The method of clause 23, wherein the transcription factor is a eukaryotic transcription factor, optionally a human transcription factor.

26. The method of any one of the preceding clauses, wherein the DNA-binding complex lacks a functional domain for activating transcription of the reporter expression product.

27. The method of any one of clauses 24 to 26, wherein the first and second components of the DNA-binding protein are DNA-binding fragments of a basic leucine zipper (bZIP), basic helix-loop helix (bHLH) or bHLH leucine zipper (bHLH-Zip) transcription factor, and optionally wherein
a) the at least one binding site is a TPA response element (TRE) having the nucleotide sequence TGACTCA (SEQ ID NO: 5) or TGAGTCA (SEQ ID NO: 6);
b) the at least one binding site is an Ebox response element having the nucleotide sequence CACGTG (SEQ ID NO: 7) or CACATG (SEQ ID NO: 8);
c) the at least one binding site is a CCAAT binding site having the nucleotide sequence ATTGCGCAAT (SEQ ID NO: 9);
d) the at least one binding site is a cAMP response element (CRE) having the nucleotide sequence TGACGTCA (SEQ ID NO: 10); or
e) the at least one binding site is a Maf recognition element (MARE) having the nucleotide sequence TGCTGA^{G}/_{C}TCAGCA (SEQ ID NO: 32) or TGCTGA^{GC}/_{CG}TCAGCA (SEQ ID NO: 33);
f) the at least one binding site is a PAP/CREB-2/PAR binding site having the nucleotide sequence TTACGTAA (SEQ ID NO: 34).

28. The method of clause 27, wherein the transcription factor is a member of the Fos/Jun subfamily of transcription factors (such as c-Jun), optionally wherein the first and second components of the DNA-binding protein each comprise an amino acid sequence that is at least 90% identical to the sequence set forth in SEQ ID NO: 47.

29. The method of clause 28, wherein the reporter expression cassette comprises a nucleotide sequence that is at least 90% identical to the sequence set forth in SEQ ID NO: 4.

30. The method of any one of the preceding clauses, wherein the first and second candidate binding partners have an identical amino acid sequence.

31. The method of any one of clauses 1 to 29, wherein the first and second candidate binding partners have different amino acid sequences.

32. The method of any one of the preceding claims, wherein the first and second candidate binding partners are capable of forming aggregates, optionally wherein the first and second candidate binding partners are capable of aggregating to form amyloids or amorphous deposits.

33. The method of any one of the preceding clauses, wherein aggregation of the first and second candidate binding partners in a human patient is associated with a disease, optionally wherein the disease is a neurodegenerative disease.

34. The method of any one of the preceding clauses, wherein the first and second candidate binding partners are amyloid peptides.

35. The method of clause 34, wherein the first and second candidate binding partners are amyloid-β (Aβ) peptides, optionally wherein the Aβ peptides comprise an amino acid sequence having the sequence of SEQ ID NO: 49.

36. The method of clause 35, wherein the first and second hybrid proteins each comprise an amino acid sequence that is at least 90% identical to the sequence set forth in SEQ ID NO: 51.

37. The method of clause 34, wherein the first and second candidate binding partners are prion proteins (PrPs).

38. The method of clause 34, wherein the first and second candidate binding partners are tau proteins.

39. The method of clause 34, wherein the first and second candidate binding partners are α-synuclein (αS) polypeptides, optionally wherein the αS polypeptides comprise an amino acid sequence having the sequence of SEQ ID NO: 53.

40. The method of clause 39, wherein the first and second hybrid proteins each comprise an amino acid sequence that is at least 90% identical to the sequence set forth in SEQ ID NO: 55.

41. The method of any one of the preceding clauses, wherein the first hybrid protein is a first fusion protein comprising the first component of the DNA-binding protein and the first candidate binding partner in the same polypeptide chain, and wherein the second hybrid protein is a second fusion protein comprising the second component of the DNA-binding protein and the second candidate binding partner in the same polypeptide chain.

42. The method of clause 41, wherein method comprises administering a fusion protein expression cassette that encodes both the first and second fusion proteins to the cell such that the cell expresses the first and second fusion proteins.

43. The method of clause 42, wherein the fusion protein expression cassette comprises a nucleotide sequence that is at least 90% identical to the sequence set forth in SEQ ID NO: 50.

44. The method of clause 42, wherein the fusion protein expression cassette comprises a nucleotide sequence that is at least 90% identical to the sequence set forth in SEQ ID NO: 54.

45. The method of any one of the preceding clauses, wherein the first and second hybrid proteins have an identical amino acid sequence.

46. The method of any one of the preceding clauses, wherein the cell is a bacterial cell, optionally an Escherichia coli cell.

47. The method of any one of clauses 1 to 45, wherein the cell is a eukaryotic cell.

48. The method of clause 47, wherein the eukaryotic cell is a mammalian cell.

49. The method of any one of the preceding clauses, wherein the test compound is a peptidic compound or a small molecule.

50. The method of clause 51, wherein the test compound is a peptidic compound.

51. The method of clause 50, wherein the compound is expressed intracellularly from a test compound expression cassette.

52. The method of clause 51, wherein the method comprises providing the test compound expression cassette to the cell.

53. The method of any one of clauses 50 to 52, wherein the method comprises administering a cross-linking agent into the cell in order to introduce a cross-link between two amino acid residues in an alpha helix of the peptidic test compound to produce a helix-constrained peptidic compound.

54. The method of clause 53, wherein the method comprises determining expression of the reporter expression product both before and after the addition of the cross-linking agent.

55. The method of clause 49 or clause 50, wherein the method comprises administering the test compound extracellularly in order to provide the cell comprising the test compound, optionally wherein an increase in expression of the reporter expression product indicates that the test compound is capable of entering the cell as well as being capable of inhibiting association between the first and second candidate binding partners.

56. The method of clause 55, wherein the test compound is a peptidic test compound, wherein the peptidic test compound comprises a helix-constrained peptide, and wherein the helix-constrained peptide comprises a cross-link between two amino acid residues.

57. The method of clause 53 or clause 56, wherein the cross-link is formed between residues i and i+4 in the peptidic test compound.

58. The method of any one of clauses 53, clause 56 or 57, wherein the cross-link is formed between cysteine residues in the peptidic test compound.

59. A method for screening for an inhibitor of association between first and second candidate binding partners, the method comprising:

providing a cell free expression system comprising:

a test compound;

a first hybrid protein comprising a first component of a DNA-binding protein linked to the first candidate binding partner;

a second hybrid protein comprising a second component of the DNA-binding protein linked to the second candidate binding partner; and

a reporter expression cassette that encodes a reporter expression product,

wherein the first and second hybrid proteins form a DNA-binding complex upon association of the first and second candidate binding partners, and wherein the reporter expression cassette comprises at least one binding site for the DNA-binding complex such that binding of the DNA-binding complex to the binding site inhibits expression of the reporter expression product; and

determining expression of the reporter expression product;

wherein an increase in expression of the reporter expression product in the presence of the test compound indicates that the test compound is capable of inhibiting association between the first and second candidate binding partners.

60. The method of any one of the preceding clauses, wherein the method further comprising carrying out an in vitro assay to confirm binding of the test compound to the first and/or second candidate binding partners.

61. The method of clause 60, wherein the in vitro assay comprises carrying out one or more of surface plasmon resonance (SPR), isothermal calorimetry and X-ray crystallography.

62. A fusion protein comprising a fusion protein comprising a component of a DNA-binding protein and a component of a DNA-binding protein and an amyloid peptide component capable of dimerization;

wherein said fusion protein forms a complex capable of binding DNA upon dimerization via the amyloid peptide component.

63. A fusion protein according to clause 62 wherein the amyloid peptide component is an amyloid-β (Aβ) peptide or an α-synuclein (αS) polypeptide.

64. The fusion protein of clause 63, wherein the Aβ peptide has the amino acid sequence set forth in SEQ ID NO: 49.

65. The fusion protein of clause 64, wherein the αS polypeptide has the amino acid sequence set forth in SEQ ID NO: 53.

66. The fusion protein of any one of clauses 63 to 65, wherein the DNA-binding component is a DNA-binding fragment of a member of the Fos/Jun subfamily of transcription factors (such as c-Jun).

67. The fusion protein of clause 66, wherein the DNA-binding component comprises an amino acid sequence that is at least 90% identical to the sequence set forth in SEQ ID NO: 47.

68. The fusion protein of clause 66 or clause 67, wherein the fusion protein comprises an amino acid sequence that is at least 90% identical to the sequence set forth in SEQ ID NO: 51.

69. The fusion protein of clause 66 or clause 67, wherein the fusion protein comprises an amino acid sequence that is at least 90% identical to the sequence set forth in SEQ ID NO: 55.

70. A fusion protein expression cassette encoding the fusion protein of any one of clauses 62 to 69. 71. A kit comprising:

a reporter expression cassette that encodes a reporter expression product; and

one or more fusion protein expression cassettes encoding a first and second fusion protein;

wherein the first fusion protein comprises a first component of a DNA-binding protein and a first candidate binding partner,

wherein the second fusion protein comprises a second component of a DNA-binding protein and a second candidate binding partner,

wherein the first and second fusion proteins form a DNA-binding complex upon association of the first and second candidate binding partners; and

wherein the reporter expression cassette comprises at least one binding site for the DNA-binding complex such that binding of the DNA-binding complex to the binding site inhibits expression of the expression product.

72. The kit of clause 71, wherein the first and second fusion proteins have an identical amino acid sequence and are both encoded by the same fusion protein expression cassette.

73. The kit of clause 71, wherein the first and second fusion proteins have non-identical amino acid sequences, wherein the kit comprises a first fusion protein expression cassette encoding the first fusion protein and a second fusion expression cassette encoding the second fusion protein.

74. The kit of any one of clauses 71 to 73, wherein the kit further comprises a test compound.

75. A cell comprising:

a reporter expression cassette that encodes a reporter expression product; and

one or more fusion protein expression cassettes encoding a first and second fusion protein;

wherein the first fusion protein comprises a first component of a DNA-binding protein and a first candidate binding partner,

wherein the second fusion protein comprises a second component of a DNA-binding protein and a second candidate binding partner;

wherein the first and second fusion proteins form a DNA-binding complex upon association of the first and second candidate binding partners; and

wherein the reporter expression cassette comprises at least one binding site for the DNA-binding complex such that binding of the DNA-binding complex to the binding site inhibits expression of the expression product.

1. A method for screening for an inhibitor of association between first and second candidate binding partners, the method comprising:
providing a cell, wherein the cell comprises:
a test compound;
a first hybrid protein comprising a first component of a DNA-binding protein linked to the first candidate binding partner;
a second hybrid protein comprising a second component of the DNA-binding protein linked to the second candidate binding partner; and
a reporter expression cassette that encodes a reporter expression product, wherein the first and second hybrid proteins form a DNA-binding complex upon association of the first and second candidate binding partners, and wherein the reporter expression cassette comprises at least one binding site for the DNA-binding complex such that binding of the complex to the binding site inhibits expression of the reporter expression product; and
determining expression of the reporter expression product in the presence of the test compound;
wherein an increase in expression of the reporter expression product in the presence of the test compound indicates that the test compound is capable of inhibiting association between the first and second candidate binding partners.

2. The method of claim 1, wherein the reporter expression product is a reporter protein, optionally wherein the reporter protein is a cell survival protein, a cell reproduction protein, a fluorescent protein, a bioluminescent protein, a protease, an enzyme that acts on a substrate to produce a colorimetric signal, a protein kinase, a transcriptional activator, or a regulatory protein such as ubiquitin.

3. The method of claim 2, wherein the reporter protein is a cell survival protein, optionally wherein the cell survival protein is an enzyme involved in synthesising compounds that are required for cell survival, or a protein that is able to inhibit action of a toxic agent.

4. The method of claim 3, wherein the cell survival protein is an exogenous cell survival protein that is able to compensate for a deficiency in an endogenous cell survival protein; and
wherein the method is performed under selection conditions such that survival of the cell is dependent upon activity of the exogenous cell survival protein.

5. The method of claim 4, wherein the cell survival protein is dihydrofolate reductase (DHFR), optionally wherein the DHFR has an amino acid sequence that is at least 80% identical to the sequence set forth in SEQ ID NO: 1.

6. The method of claim 1, wherein the reporter expression cassette comprises between 1 and 5, between 1 and 10, between 1 and 15, between 1 and 20, between 5 and 10, between 5 and 15, between 5 and 20, between 10 and 15, between 10 and 20, between 10 and 18 or between 12 and 16 binding sites.

7. The method of claim 2, wherein some or all of the binding site(s) are located in the protein coding sequence of the reporter expression cassette.

8. The method of claim 1, wherein the first and second components of the DNA-binding protein have an identical amino acid sequence.

9. The method of claim 1, wherein the first and second components of the DNA-binding protein are DNA-binding fragments of a eukaryotic transcription factor, optionally a human transcription factor.

10. The method of claim 9, wherein the first and second components of the DNA-binding protein are DNA-binding fragments of a basic leucine zipper (bZIP), basic helix-loop helix (bHLH) or bHLH leucine zipper (bHLH-Zip) transcription factor, and optionally wherein
a) the at least one binding site is a TPA response element (TRE) having the nucleotide sequence TGACTCA (SEQ ID NO: 5) or TGAGTCA (SEQ ID NO: 6);
b) the at least one binding site is an Ebox response element having the nucleotide sequence CACGTG (SEQ ID NO: 7) or CACATG (SEQ ID NO: 8);
c) the at least one binding site is a CCAAT binding site having the nucleotide sequence ATTGCGCAAT (SEQ ID NO: 9);
d) the at least one binding site is a cAMP response element (CRE) having the nucleotide sequence TGACGTCA (SEQ ID NO: 10);
e) the at least one binding site is a Maf recognition element (MARE) having the nucleotide sequence TGCTGA^{G}/_{C}TCAGCA (SEQ ID NO: 32) or TGCTGA^{GC}/_{CG}TCAGCA (SEQ ID NO: 33); or
f) the at least one binding site is a PAP/CREB-2/PAR binding site having the nucleotide sequence TTACGTAA (SEQ ID NO: 34).

11. The method of claim 1, wherein the first and second candidate binding partners are capable of forming protein aggregates, optionally wherein the first and second candidate binding partners are amyloid peptides.

12. The method of claim 11, wherein
a) the first and second candidate binding partners are amyloid-β (Aβ) peptides, optionally wherein the Aβ peptides comprise an amino acid sequence having the sequence of SEQ ID NO: 49; or
b) the first and second candidate binding partners are α-synuclein (αS) polypeptides, optionally wherein the αS polypeptides comprise an amino acid sequence having the sequence of SEQ ID NO: 53.

13. A fusion protein comprising a component of a DNA-binding protein and an amyloid peptide component capable of dimerization;
wherein said fusion protein forms a complex capable of binding DNA upon dimerization via the amyloid peptide component.

14. The fusion protein of claim 13, wherein the amyloid peptide component is:
a) an amyloid-β (Aβ) peptide, optionally wherein the Aβ peptide has the amino acid sequence set forth in SEQ ID NO: 49; or
b) an α-synuclein (αS) polypeptide, optionally wherein the αS polypeptide has the amino acid sequence set forth in SEQ ID NO: 53.

15. The fusion protein of claim 14, wherein the DNA-binding component comprises an amino acid sequence that is at least 90% identical to the sequence set forth in SEQ ID NO: 47, optionally wherein the fusion protein comprises an amino acid sequence that is at least 90% identical to the sequence set forth in SEQ ID NO: 51 or 55.

16. A fusion protein expression cassette encoding the fusion protein of claim 13.

17. A kit comprising:
a reporter expression cassette that encodes a reporter expression product; and
one or more fusion protein expression cassettes encoding a first and second fusion protein;
wherein the first fusion protein comprises a first component of a DNA-binding protein and a first candidate binding partner,
wherein the second fusion protein comprises a second component of a DNA-binding protein and a second candidate binding partner,
wherein the first and second fusion proteins form a DNA-binding complex upon association of the first and second candidate binding partners; and
wherein the reporter expression cassette comprises at least one binding site for the DNA-binding complex such that binding of the DNA-binding complex to the binding site inhibits expression of the expression product.

18. (canceled)
